SEQUENCE LISTING (155 sequences)

SEQ ID NO: 1 - protein, 15 aa, Homo sapiens
PQVGKKLWQLCWIMT

SEQ ID NO: 2 - protein, 15 aa, Homo sapiens
CLRLFTRNHFAVILL

SEQ ID NO: 3 - protein, 15 aa, Homo sapiens
MYQKSPGRLVDGSSW

SEQ ID NO: 4 - protein, 15 aa, Homo sapiens
HEAGLGLHLEGTLWP

SEQ ID NO: 5 - protein, 15 aa, Homo sapiens
GPHHRTLELPTEPDP

SEQ ID NO: 6 - protein, 15 aa, Homo sapiens
PSTGCPPPGKALLQG

SEQ ID NO: 7 - protein, 15 aa, Homo sapiens
FLHRHSEAAGAYHRL

SEQ ID NO: 8 - protein, 15 aa, Homo sapiens
QLRPLPGHQWQARKE

SEQ ID NO: 9 - protein, 15 aa, Homo sapiens
GRATLQRGGFGAGAG

SEQ ID NO: 10 - protein, 15 aa, Homo sapiens
VSRHVALGLPHLGSL

SEQ ID NO: 11 - protein, 15 aa, Homo sapiens
QWAPTSGSSPTQPWE

SEQ ID NO: 12 - protein, 15 aa, Homo sapiens
EEPLDWSQSLSQTHQ

SEQ ID NO: 13 - protein, 15 aa, Homo sapiens
TKERGFEGTLKIHRG

SEQ ID NO: 14 - protein, 15 aa, Homo sapiens
QPSLAAGVLRPRLAG

SEQ ID NO: 15 - protein, 15 aa, Homo sapiens
GLSAAQITGREPRHP

SEQ ID NO: 16 - protein, 15 aa, Homo sapiens
RTGLQLCRRARRPQR

SEQ ID NO: 17 - protein, 15 aa, Homo sapiens
VVCQEGWSGPLLAQR

SEQ ID NO: 18 - protein, 15 aa, Homo sapiens
LVPASLGQSQPGITA

SEQ ID NO: 19 - protein, 15 aa, Homo sapiens
GLGAQDGRGDRVRAG

SEQ ID NO: 20 - protein, 15 aa, Homo sapiens
ARLRRQPLGHRAGTG

SEQ ID NO: 21 - protein, 15 aa, Homo sapiens
EKQVSMARLAPQGSQ

SEQ ID NO: 22 - protein, 15 aa, Homo sapiens
WRVRGCGRPLGAGCC

SEQ ID NO: 23 - protein, 15 aa, Homo sapiens
AEATAGREPPRPRPP

SEQ ID NO: 24 - protein, 15 aa, Homo sapiens
ALGAAPRVPAPTAPA

SEQ ID NO: 25 - protein, 15 aa, Homo sapiens
SRAPRPPRHPPAAPT

SEQ ID NO: 26 - protein, 15 aa, Homo sapiens
SARTYGLATRTCGDW

SEQ ID NO: 27 - protein, 15 aa, Homo sapiens
GLGAELCLQVQVLRD

SEQ ID NO: 28 - protein, 15 aa, Homo sapiens
LVRHPAPAAATRHSD

SEQ ID NO: 29 - protein, 15 aa, Homo sapiens
SGTRHTGAASTTNPH

SEQ ID NO: 30 - protein, 15 aa, Homo sapiens
QTCASPSRTPKRPSQ

SEQ ID NO: 31 - protein, 15 aa, Homo sapiens
SMPLSLQPTLLPDPS

SEQ ID NO: 32 - protein, 15 aa, Homo sapiens
LTPGASTTSASTGTD

SEQ ID NO: 33 - protein, 15 aa, Homo sapiens
MLGDYIFSMASVTSC

SEQ ID NO: 34 - protein, 15 aa, Homo sapiens
PLPPQVPTAGATGKT

SEQ ID NO: 35 - protein, 15 aa, Homo sapiens
FASAASGTRHTGAAS

SEQ ID NO: 36 - protein, 15 aa, Homo sapiens
TTNPHQTCASPSRTP

SEQ ID NO: 37 - protein, 15 aa, Homo sapiens
KRPSQSMPLSLQPTL

SEQ ID NO: 38 - protein, 15 aa, Homo sapiens
LPDPSLTPGASTTSA

SEQ ID NO: 39 - protein, 15 aa, Homo sapiens
STGTDMLGDYIFSMA

SEQ ID NO: 40 - protein, 15 aa, Homo sapiens
EIHLGTIRKLFCCSV

SEQ ID NO: 41 - protein, 15 aa, Homo sapiens
EKMTNVSRGRAPCCV

SEQ ID NO: 42 - protein, 15 aa, Homo sapiens
PAPPHCLPSAPLAAF

SEQ ID NO: 43 - protein, 15 aa, Homo sapiens
VCQCLTHCLIHTSML

SEQ ID NO: 44 - protein, 15 aa, Homo sapiens
WPPPPPAPVSPTTTS

SEQ ID NO: 45 - protein, 15 aa, Homo sapiens
GNPRALRAGGHHARQ

SEQ ID NO: 46 - protein, 15 aa, Homo sapiens
DHRGVPQRGPVLRGQ

SEQ ID NO: 47 - protein, 15 aa, Homo sapiens
EVRAVAAPRGHLRGA

SEQ ID NO: 48 - protein, 15 aa, Homo sapiens
AGAAHGAGGRPVRRA

SEQ ID NO: 49 - protein, 15 aa, Homo sapiens
PPLHAHAWAPGAGAG

SEQ ID NO: 50 - protein, 15 aa, Homo sapiens
RAAGGRQVRGGGPRA

SEQ ID NO: 51 - protein, 15 aa, Homo sapiens
VPTAGATGKTFASAA

SEQ ID NO: 52 - protein, 15 aa, Homo sapiens
ERQQQQSGPASLAGF

SEQ ID NO: 53 - protein, 15 aa, Homo sapiens
TPSGTTCHCIVDSCG

SEQ ID NO: 54 - protein, 15 aa, Homo sapiens
SRPCSTAAQPACHLQ

SEQ ID NO: 55 - protein, 15 aa, Homo sapiens
SRMRELARALGGSST

SEQ ID NO: 56 - protein, 15 aa, Homo sapiens
LMGGRAEKPPGGGLS

SEQ ID NO: 57 - protein, 15 aa, Homo sapiens
PWTIATSIPRAVAAQ

SEQ ID NO: 58 - protein, 15 aa, Homo sapiens
PRRRQPCRQPPNQLT

SEQ ID NO: 59 - protein, 15 aa, Homo sapiens
TVPPSSPSGLAAPRH

SEQ ID NO: 60 - protein, 15 aa, Homo sapiens
AAVMSWMRGRTSVHA

SEQ ID NO: 61 - protein, 15 aa, Homo sapiens
PILTPAQSVAACRPS

SEQ ID NO: 62 - protein, 15 aa, Homo sapiens
WQAQSWMKSRTMMRL

SEQ ID NO: 63 - protein, 15 aa, Homo sapiens
EREVIQERNRNFRQN

SEQ ID NO: 64 - protein, 15 aa, Homo sapiens
IHSFIHWIVYHCCTI

SEQ ID NO: 65 - protein, 15 aa, Homo sapiens
RIDKHCSSTPFSNYV

SEQ ID NO: 66 - protein, 15 aa, Homo sapiens
TLFYCSWFLNVFHSF

SEQ ID NO: 67 - protein, 15 aa, Homo sapiens
LVPGATQSQWTLEGR

SEQ ID NO: 68 - protein, 15 aa, Homo sapiens
QYSKAAPGEGEGGSG

SEQ ID NO: 69 - protein, 15 aa, Homo sapiens
PRAREELVPDQRREE

SEQ ID NO: 70 - protein, 15 aa, Homo sapiens
GCCEEVFCRVSCIIH

SEQ ID NO: 71 - protein, 15 aa, Homo sapiens
GQFYEALEGTMDRSW

SEQ ID NO: 72 - protein, 15 aa, Homo sapiens
GDRLGAGAGAGTDGR

SEQ ID NO: 73 - protein, 15 aa, Homo sapiens
GGAGGGDRDGFRALC

SEQ ID NO: 74 - protein, 15 aa, Homo sapiens
VLVGGGGAVPGSFGP

SEQ ID NO: 75 - protein, 15 aa, Homo sapiens
DARPPHGAAGGWGSR

SEQ ID NO: 76 - protein, 15 aa, Homo sapiens
GVPGSSAEAPAEAGD

SEQ ID NO: 77 - protein, 15 aa, Homo sapiens
AEGPASTRGAAGIGG

SEQ ID NO: 78 - protein, 15 aa, Homo sapiens
GGLGHGGGPGARPRA

SEQ ID NO: 79 - protein, 15 aa, Homo sapiens
LAPATSAGGEPGAAG

SEQ ID NO: 80 - protein, 15 aa, Homo sapiens
PRRGGRRERCLPPCR

SEQ ID NO: 81 - protein, 45 aa, Homo sapiens
PQVGKKLWQLCWIMTCLRLFTRNHFAVILLMYQKSPGRLVDGSSW

SEQ ID NO: 82 - protein, 39 aa, Homo sapiens
HEAGLGLHLEGTLWPGPHHRTLELPTEPDPGAPGGRPRR

SEQ ID NO: 83 - protein, 56 aa, Homo sapiens
PSTGCPPPGKALLQGFLHRHSEAAGAYHRLQLRPLPGHQWQARKEDRWRLERHDRG

SEQ ID NO: 84 - protein, 29 aa, Homo sapiens
GRATLQRGGFGAGAGRAVRSGRGAADRHR

SEQ ID NO: 85 - protein, 30 aa, Homo sapiens
VSRHVALGLPHLGSLQWAPTSGSSPTQPWE

SEQ ID NO: 86 - protein, 79 aa, Homo sapiens
EEPLDWSQSLSQTHQTKERGFEGTLKIHRGQPSLAAGVLRPRLAGGLSAAQITGREPRHPRTGLQLCRRARRPQRVCGE

SEQ ID NO: 87 - protein, 39 aa, Homo sapiens
VVCQEGWSGPLLAQRLVPASLGQSQPGITASLCPPQCRE

SEQ ID NO: 88 - protein, 37 aa, Homo sapiens
GLGAQDGRGDRVRAGARLRRQPLGHRAGTGRRRLQHE

SEQ ID NO: 89 - protein, 17 aa, Homo sapiens
EKQVSMARLAPQGSQET

SEQ ID NO: 90 - protein, 77 aa, Homo sapiens
WRVRGCGRPLGAGCCAEATAGREPPRPRPPALGAAPRVPAPTAPASRAPRPPRHPPAAPTSARTYGLATRTCGDWCT

SEQ ID NO: 91 - protein, 33 aa, Homo sapiens
GLGAELCLQVQVLRDLVRHPAPAAATRHSDARQ

SEQ ID NO: 92 - protein, 95 aa, Homo sapiens
PLPPQVPTAGATGKTFASAASGTRHTGAASTTNPHQTCASPSRTPKRPSQSMPLSLQPTLLPDPSLTPGASTTSASTGTDMLGDYIFSMASVTSC

SEQ ID NO: 93 - protein, 90 aa, Homo sapiens
VPTAGATGKTFASAASGTRHTGAASTTNPHQTCASPSRTPKRPSQSMPLSLQPTLLPDPSLTPGASTTSASTGTDMLGDYIFSMASVTSC

SEQ ID NO: 94 - protein, 67 aa, Homo sapiens
EIHLGTIRKLFCCSVEKMTNVSRGRAPCCVPAPPHCLPSAPLAAFVCQCLTHCLIHTSMLMTNTYTS

SEQ ID NO: 95 - protein, 21 aa, Homo sapiens
WPPPPPAPVSPTTTSSSRSLA

SEQ ID NO: 96 - protein, 95 aa, Homo sapiens
GNPRALRAGGHHARQDHRGVPQRGPVLRGQEVRAVAAPRGHLRGAAGAAHGAGGRPVRRAPPLHAHAWAPGAGAGRAAGGRQVRGGGPRALQGAR

SEQ ID NO: 97 - protein, 18 aa, Homo sapiens
ERQQQQSGPASLAGFSVH

SEQ ID NO: 98 - protein, 150 aa, Homo sapiens
TPSGTTCHCIVDSCGSRMRELARALGGSSTLMGGRAEKPPGGGLSPWTIATSIPRAVAAQPRRRQPCRQPPNQLTTVPPSSPSGLAAPRHAAVMSWMRGRTSVHAPILTPAQSVAACRPSWQAQSWMKSRTMMRLSRPCSTAAQPACHLQ

SEQ ID NO: 99 - protein, 60 aa, Homo sapiens
EREVIQERNRNFRQNIHSFIHWIVYHCCTIRIDKHCSSTPFSNYVTLFYCSWFLNVFHSF

SEQ ID NO: 100 - protein, 16 aa, Homo sapiens
LVPGATQSQWTLEGRM

SEQ ID NO: 101 - protein, 33 aa, Homo sapiens
QYSKAAPGEGEGGSGPRAREELVPDQRREEEGE

SEQ ID NO: 102 - protein, 34 aa, Homo sapiens
GCCEEVFCRVSCIIHGQFYEALEGTMDRSWWTVL

SEQ ID NO: 103 - protein, 142 aa, Homo sapiens
GVPGSSAEAPAEAGDGGAGGGDRDGFRALCVLVGGGGAVPGSFGPDARPPHGAAGGWGSRGDRLGAGAGAGTDGRAEGPASTRGAAGIGGGGLGHGGGPGARPRALAPATSAGGEPGAAGPRRGGRRERCLPPCRPRRGRPG

SEQ ID NO: 104 - protein, 26 aa, Homo sapiens
AASSCMGPWCSSSLSSSTWWPSPSPH

SEQ ID NO: 105 - protein, 128 aa, Homo sapiens
CRRERCSWPRWPRWRWPGSPLRRRVPRAATCRGVPAPAAPAATCPTSATAAWCAPPARASPVAALWTRLAARAWSACAAYAAAAGRTPCVAPTGTPMPTCARCRRPAAARCSSPGRPCASCRRAPARW

SEQ ID NO: 106 - protein, 43 aa, Homo sapiens
KQHVIYSNKKYISFWAQSINPVTEKRIQVEQTRDEDLDTDSLD

SEQ ID NO: 107 - protein, 21 aa, Homo sapiens
CHRQGDRRLGETCRLIFMSKH

SEQ ID NO: 108 - protein, 170 aa, Homo sapiens
STASASRRWMGTSEHPNAPRWAAASGAGIWEDTAGSAGLLQGWPKASAAARSGTCPASSLPPSGTSLATLAVMTSWLIGPGPAHSSHACQKLEGCWRPIELMGAAGAWPSAPASPEPKGTSWRPHGATRLPRDGSACTFETPVSFNVHIPGDHTCLLLLILASRFTLTLY

SEQ ID NO: 109 - protein, 23 aa, Homo sapiens
EKEEPLDQWESKVLRDAMAPKVL

SEQ ID NO: 110 - protein, 63 aa, Homo sapiens
LWALTLTSVCKFKRMEKSVWSAPQWDGTQSPRCSGELPRERSFHLHQSPGILMKKVCSLWLLQ

SEQ ID NO: 111 - protein, 13 aa, Homo sapiens
EKYVEVSRRPVRA

SEQ ID NO: 112 - protein, 27 aa, Homo sapiens
EWQYFREASDSMPRFTPRISSLLPPWA

SEQ ID NO: 113 - protein, 54 aa, Homo sapiens
YIQTRIEEMDHLNISFQEMEQEISSLLMKTQATYRPPRGWTGKKNPFTSFELKL

SEQ ID NO: 114 - protein, 201 aa, Homo sapiens
RSAEGAGARLGDDEGTGVSDERDAVLGRRGAGAPAGRPLLPLLSRRLPGPGAPWRRGPPGPLGHAAQLSVPGGGRLRAGHSRAAARGRRDGDSSAGPGQETRVRASLVHERGVRRPGAAPEGREGVRQHQSPRYGGLREREDLLWGRDGGVREYECVVRVREYWGPGRPGPHGSGKNVGDCLEIDFEPDENKEWKASVLPI

SEQ ID NO: 115 - protein, 38 aa, Homo sapiens
GAVDHSLPEEADKAEGSPWLSKASLQAKEASTSSGEKW

SEQ ID NO: 116 - protein, 32 aa, Homo sapiens
ICSGPPASMPASSYPTSPSTASLGCCSSLLLS

SEQ ID NO: 117 - protein, 140 aa, Homo sapiens
ASGPGPRPSERAQATGHAAILPREALLGARLGLGPGNQNSVPYPAGALLEPSWGVVAGNPTGTSALPGLVPSWTACTCQPPTCFLKATLAHTRMRPVQPAKGQSGGAASCQCPPQLCLPFSPARFNQNAMLCKSLLLGGG

SEQ ID NO: 118 - protein, 30 aa, Homo sapiens
LISQMNYLWAPRMCTDSSPPGKTLRQWRQI

SEQ ID NO: 119 - protein, 23 aa, Homo sapiens
LKDMTIVLMTTWKLEMEPVKIAL

SEQ ID NO: 120 - protein, 45 aa, Homo sapiens
LKPWSHSCDTSMGRSARAKSKRAWRLQHNASGPTRCWSHPVMRWR

SEQ ID NO: 121 - protein, 41 aa, Homo sapiens
RLMMTPVPMTSSSGRFTSAAWMTCSSFWPARLLRSNGAYMC

SEQ ID NO: 122 - protein, 18 aa, Homo sapiens
LPLHQRTSSSTASWSPGW

SEQ ID NO: 123 - protein, 22 aa, Homo sapiens
LPSLGARATTTQPVKTYVAEGQ

SEQ ID NO: 124 - protein, 22 aa, Homo sapiens
LQVQTKKKKGRRGRKEKQRTNL

SEQ ID NO: 125 - protein, 20 aa, Homo sapiens
LRAVRPGPEKPAGSLCWEQW

SEQ ID NO: 126 - protein, 61 aa, Homo sapiens
GGGWLLGAPGALWTSPPAQHHPVLPAAPLWPLGSWLSLEPVLRAVRPGPEKPAGSLCWEQW

SEQ ID NO: 127 - protein, 12 aa, Homo sapiens
LSNGGRLLQIWF

SEQ ID NO: 128 - protein, 115 aa, Homo sapiens
LVPDSPSSLAGTGTVCQGKWTQPWLAASTSQAWHPAPPWPRNKGLGIATAKATVHNEATAVAATRTPAGHPAPRGCPCSPVRRATWEPTTMMTTGWTGLCLPPVNPSRVSSSSLE

SEQ ID NO: 129 - protein, 30 aa, Homo sapiens
LVPSPSRNVPAELSMRRTSSRFTPSSFLKE

SEQ ID NO: 130 - protein, 29 aa, Homo sapiens
MKMFDHTSETDFACMICCPQIKYKDLSNH

SEQ ID NO: 131 - protein, 17 aa, Homo sapiens
NLFPVHAMCLNAAMTVF

SEQ ID NO: 132 - protein, 20 aa, Homo sapiens
NNWKESMMLFPVNMSAADEC

SEQ ID NO: 133 - protein, 70 aa, Homo sapiens
AICFLEEKAKQGLLKSLSPIWAVSPTWHIGRASSWSISTTTMRGTSASRQLAERRACAHMTLPPLCSTST

SEQ ID NO: 134 - protein, 35 aa, Homo sapiens
SGRERMRKARRGRTPCPGPRPTGCVPSTRCGCSCR

SEQ ID NO: 135 - protein, 36 aa, Homo sapiens
LSLRKLQRRLGTLRPSHRMRRCCSSMATTNKQLWAT

SEQ ID NO: 136 - protein, 65 aa, Homo sapiens
GPSGSGCPGSSVAMPRPTAAMAACPGRSCSSPPSRCSEGGMWWPLSSAVSCTTASCGLPCRRQPC

SEQ ID NO: 137 - protein, 58 aa, Homo sapiens
SRMTSSTPVCSTSWTRSSLPCWQIPPVASFTWRLPSSPVGGTSRQMPHRKSCETLCAR

SEQ ID NO: 138 - protein, 13 aa, Homo sapiens
SWDTENWSRDKWQ

SEQ ID NO: 139 - protein, 24 aa, Homo sapiens
TCRLPKCMGCFQGCIYFKTSLQHY

SEQ ID NO: 140 - protein, 49 aa, Homo sapiens
QTVLGRIHQRCQERPLHRPPAAVRPQQCQSVLSERPLDSCRETQACRAV

SEQ ID NO: 141 - protein, 31 aa, Homo sapiens
VTFWTEMPIMKEMEIIPICTRDTRRQKEELQ

SEQ ID NO: 142 - protein, 20 aa, Homo sapiens
WAQASVLLMIPTDMQSMRTM

SEQ ID NO: 143 - protein, 19 aa, Homo sapiens
WSAQRSSETWSKPLTPCSL

SEQ ID NO: 144 - protein, 47 aa, Homo sapiens
WTRPWPSTLGMGQSWPSKKISTQPCQSPSGSAHSATRTWSLRPRRMA

SEQ ID NO: 145 - protein, 39 aa, Homo sapiens
WTSGGGLPTLAPPSTSECPSPASRRSRSFWSPTASAMRP

SEQ ID NO: 146 - protein, 57 aa, Homo sapiens
GVLLGPTSHGIGGDSRPRPRFRLRLQDQEEAFRAAEGEGVPGGRPAGGCVPVGRSPL

SEQ ID NO: 147 - protein, 142 aa, Homo sapiens
GSHGLPLTPLPPACSAGIGSCWRQPRPPRTAGFPSREDPEGEAARPAAGSSLCLGLLGSLEPLHRWHHLGAETRRGGAHGRGLLPLHRGLPPAEASQLLRPLRVGGPAGEVASPGQRAPLLAPGAGHTDTRHQLPQRDAAEQ

SEQ ID NO: 148 - protein, 41 aa, Homo sapiens
ALLQEGVPLPRFPGQIPGFPEAGQTSSCPHHQNHPPTGGTI

SEQ ID NO: 149 - protein, 112 aa, Homo sapiens
ARGAGGSRARRPAVRHGQHPEEADWQSRRRQAAGKGRAAAGPDPGGRRGQAEPTAGLCGGGGGRRRHRASGRRAGRGEPCRPEEPGQRAVPKRAVRRGGRQVLGGNRAPGAS

SEQ ID NO: 150 - protein, 103 aa, Homo sapiens
GVQREVSQQPQARHQGAPGHHRGQGTPGSRWESPLDQALEEGGPGREAAAASSPHHGTCQEDLGQGRPCPSQQPQQPEASPHGPQQSRCNPGAQGSWGRGGGR

SEQ ID NO: 151 - protein, 32 aa, Homo sapiens
EDWPICRALQPAVEHQRRPFLVQSEPGSHPHV

SEQ ID NO: 152 - protein, 48 aa, Homo sapiens
FLLSTSLTSSTLREPGSRDSTPSMKSSPGSWSPPSPSQPTSSDHLKKK

SEQ ID NO: 153 - protein, 45 aa, Homo sapiens
KRPIKQWARRLQKGSLMKKEQTVKQWKKRNLRATYDATPRREESQ

SEQ ID NO: 154 - protein, 68 aa, Homo sapiens
TLGRRRRKGAAAVKDLTPLPLIGSSLGPGISCAAPSIALSTGSGGSRLTTWDRPLTVAHGSYPIPTEY

SEQ ID NO: 155 - protein, 34 aa, Homo sapiens
LVTGATLATTLLPSRPHRMLVTGSSEPRQLQWLD

(19) United States
(12) Patent Application Publication
(10) Pub. No.: US 2021/0223257 A1
(43) Pub. Date: Jul. 22, 2021

(54) METHOD AND SYSTEM FOR DETERMINING MICROSATELLITE INSTABILITY

(71) Applicant: Arizona Board of Regents on behalf of Arizona State University, Scottsdale, AZ (US)

(72) Inventors: Stephen Albert JOHNSTON, Tempe, AZ (US); Phillip STAFFORD, Phoenix, AZ (US); Jian ZHANG, Peoria, AZ (US); Luhui SHEN, Tempe, AZ (US)

(21) Appl. No.: 17/249,863

(22) Filed: Mar. 16, 2021

Related U.S. Application Data
(63) Continuation of application No. PCT/US2019/052822, filed on Sep. 25, 2019.
(60) Provisional application No. 62/736,314, filed on Sep. 25, 2018.

(51) Int. Cl. G01N 33/68; A61K 39/00; A61K 45/06

(52) U.S. Cl. CPC G01N 33/6845; G01N 33/6854; A61K 39/39; A61K 45/06; A61K 39/0011

(57) ABSTRACT

Disclosed herein are methods and systems for determining microsatellite instability. In some embodiments, the disclosed methods and systems are used for determining whether a cancer patient has high microsatellite instability (MSI-H). MSI-H patients have remarkably good responses to immunotherapy, such as checkpoint inhibitors immunotherapy. Therefore, the disclosed methods and systems can be used for identifying MSI-H and thus, patients as candidates for immunotherapy. In turn, the disclosed methods and systems can be used to predict responsiveness to immunotherapy. In some embodiments, the methods further include providing an immunotherapy to the MSI-H patient. Also disclosed are vaccines and compositions.

### INCORPORATION BY REFERENCE TO RELATED APPLICATIONS

This application is continuation of PCT Application No. PCT/US2019/052822, filed Sep. 25, 2019, which claims the benefit of priority to U.S. Provisional Application No. 62/736,314, filed Sep. 25, 2018. The disclosures of the above-referenced applications are hereby incorporated by reference in their entireties.

### ACKNOWLEDGEMENT OF GOVERNMENT SUPPORT

This invention was made with government support under R21 CA220150 awarded by the National Institutes of Health. The government has certain rights in the invention.

### FIELD

This disclosure relates to cancer and in particular, to methods and systems for determining microsatellite instability (MSI)/MisMatch Repair (MMR) status, predicting outcomes to immunotherapeutic treatments and vaccines for MSI-High/MMR patients.

### BACKGROUND

CPIs are now a $4B/year market and growing. However, for most cancers the clinical response rate is at best 20-40%. Given the cost of these treatments ($100K+) and that there can be serious side-effects there is incentive to determine who will and will not respond to CPI treatment. A number of markers have been explored including PDL-1 levels, TMB (total mutational burden), T-cell infiltrate and neoantigens amounts. However, the best indicator of response is that the tumor is MSI-High (MSI-H)/MisMatch Repair MMR). The high rate of response by MSI-H patients, regardless of tumor type, was the basis for the historic approval of Keytruda for any cancer with an MSI-H genotype.

Microsatellites are runs of repeats of one or a set of nucleotides. For example, 10 A nucleotides in a row. DNA polymerase is much more likely to lose register and insert or delete (indel) a nucleotide in these runs during replication. In normal cells the indels are sensed and repaired. In MSI tumors, there is a defect in this repair system. An indel results in the coding sequence being “frameshifted” downstream of the run. The result is the encoding of a junk peptide sequence until the ribosome runs into a stop codon. The MSI-H condition can also induce more aberrant splicing of mRNA that generate FS transcripts that encode FS peptides. Over 200 proteins participate in the splicing process and 21 of them contain MS, which can be affected by MSI. INDELs of MS in introns can also cause aberrant splicing. Overall, MSI-H tumors express more FS peptides than other tumors. These frameshift peptides (FSP) are highly immunogenic cancer neoantigens and the basis for the anti-tumor immune responses in MSI-H patients that leads better responses to the CPI treatment. Some cancers (endometrial 28%, stomach 22%, colon 17%) have relatively high incidence of MSI-H cancers. However, most cancers have 0-9% MSI-H. Patients with these cancers are not routinely tested.

The MSI status can currently be assessed in several ways. The current gold standards for MSI-H/dMMR diagnosis are PCR analysis of five or six microsatellite markers in the tumor genome and IHC analysis of expression of MMR proteins (McNeil et al., J. Vet. Intern. Med. 21:1034-1040, 2007)). Another recently developed method is to sequence the total tumor coding sequence to tabulate all indels (Salipante, et al., Clin Chem 60, 1192-1199, doi:10.1373/clinchem.2014.223677 (2014)). All of the three methods require sufficient amount of tumor biopsy which in 20-30% of the time may not be available and is often contaminated by normal adjacent tissues. It may take weeks to a month from scheduling a biopsy to finishing the diagnosis report. And for the sequencing approach, it may take longer and be more expensive. None of these three methods directly evaluates the immune response to the FS neoantigens in patients, which is most directly related with the outcome of the CPI treatment. As such, a need exists to develop methods and systems which address these shortcomings. Recently, it has been reported that MSI status can be determined by applying multiple sequence analyses of circulating tumor DNA (Georgeadis, et al., Clinical Cancer Research. 2019. DOI: 10.1158/1078-0432.CCR-19-1372). However, the sample size of MSS was very small and the clinical relevance of this approach remains to be demonstrated.

### SUMMARY

Disclosed herein are methods and systems for determining MSI status. The disclosed methods and systems are advantageous for they are simple, non-invasive techniques capable of determining with high accuracy MSI status. The disclosed methods and systems directly assay for the clinically relevant agent—frameshift antigens. The methods only require a small biological sample, such as a single droplet of blood, instead of a tumor tissue sample. Further, instead of sampling 6 MS, the disclosed method and system assay for all relevant frameshift peptides. Additionally, as the disclosed methods and systems are inexpensive and simple it opens testing for MSI even in cancer types where it is rare. This assay allows defining a vaccine for MSI-H patients that would be a companion to the diagnostic and to treatment with CPIs.

In some embodiments, the disclosed methods and systems are used for determining whether a cancer patient has high microsatellite instability (MSI-H). MSI-H patients have remarkably high response rates (>50%) to immunotherapy. Therefore, the disclosed methods and systems can be used for identifying MSI-H and thus, patients as candidates for immunotherapy. The disclosed methods and systems can be used to indicate which FS peptides are good candidates for vaccines. As such, vaccines including identified FS peptides are disclosed as well for therapeutic vaccines for MSI-H patients.

The foregoing and other features will become more apparent from the following detailed description of several embodiments, which proceeds with reference to the accompanying figures.

### BRIEF DESCRIPTION OF THE DRAWINGS

Those of skill in the art will understand that the drawings, described below, are for illustrative purposes only. The drawings are not intended to limit the scope of the present teachings in any way.

FIGS. 1A-1C. Use of FSP arrays to distinguish MSI status. FIG. 1A). The FSP that would result from INDELS in microsatellite regions in coding sequences were used (approximately 14K total of 400K on array) to classify MSI-H from non-cancer controls, MSI-Low and MSI-Stable. 100% accuracy in leave-one out analysis. The 100 best peptides by two way t-test were used. FIG. 1B). Same procedure as in FIG. 1A was used, but using all the 400K FSPs. The best 91 peptides for classification were all from exon mis-splicing and exon 1 mis-initiation FSPs. 100% accuracy in leave-one out analysis. FIG. 1C). Using all 400K FSP and ANOVA analysis for significance, peptides were chosen that could separate all 3 states of MSI—MSI-H, MSI-L and MSI-S. The peptides provided in Table 1 could distinguish all 3 from each other with high accuracy.

FIG. 2A is a schematic of a mask-based patterned synthesis of peptides performed on 200-mm silicon wafers with thermal oxide coating, starting with an aminosilane-glycine monolayer and building peptides through cycles of patterned acid formation in a photoresist removing Boc groups from the N-terminal amines of nascent peptides and coupling of the next amino acid. The same process is used to make both the Immunosignature and FSP arrays, but the wafer surfaces are different.

FIG. 2B is a schematic and digital image. The schematic illustrates a FSB wafer being diced into microscope slide-sized regions (75×25 mm), each of which contains 16 arrays of 392,000, 8-μm features. Samples are individually applied to each array via a commercially available gasket system and scanned on a laser scanner. The digital image is of the array (at 800× magnification) of serum applied to the array and antibody binding detected with a fluorescent secondary antibody.

FIG. 2C illustrates two types of arrays that can be used for the disclosed methods: 1) Immunosignature (IMS) arrays are created with 10 k-1M peptides that are chosen from random sequence space. The peptides are 8-30 amino acids long are spaced (<3 nm) to create avidity binding of antibodies for lower affinity epitopes in the peptides. 2) Frameshift peptide (FSP) arrays are created with up to 400,000 peptides that are chosen from the 220,000 possible FS peptides resulting from indels in microsatellites (inserted in the DNA or only the RNA) or from mis-splicing of exons in forming the RNA. The peptides are 8-30 amino acids long and are spaced further than in IMS to enhance high affinity, cognate binding of antibodies.

FIG. 3 is a heatmap showing immunosignature results from 10 MSI-H (left), 10 MSI-L (middle) and 10 MSI-S (right)+18 controls of unknown MSI status (far right). Cross-validation accuracy was 82% correct, mis-calling 2 MSI-S patients as MSI-L, 1 control as MSI-H and 4 MSI-H as MSI-L.

FIG. 4 is a Principal Component Analysis of Immunosignature data showing grouping by status.

FIG. 5 is a heatmap of FS peptide array data from the 30 MSI patients plus 18 controls. This heatmap shows FS 400 peptides that distinguish the MSI-H from MSI-L patients with 100% accuracy using leave-one-out cross-validation.

FIG. 6 shows PCA of FS data. Samples from FIG. 3 were analyzed using PCA, a method to visualize the separation between data for each sample. As shown, there is considerable heterogeneity from sample to sample in each class other than controls, but the distance between each class is sufficient to support high classification accuracy.

FIG. 7 illustrates the Cumulative score from FS array data. Median normalized fluorescent intensity scores from the 400 peptides shown in FIG. 3 were summed and plotted on a line graph to illustrate the total relative fluorescence intensity compared to healthy non-MSI-scored people. While this cumulative score could not discriminate between MSI-H and MSI-L, it is extremely sensitive and specific to anyone with mismatch repair vs. non. Note that patient 32 and 34 (middle of graph) were mis-identified by IMS as MSI-H.

FIG. 8. Demonstrates the diagnostic potential of FSPs provided in Table 3.

### DETAILED DESCRIPTION

In the following detailed description, reference is made to the accompanying drawings which form a part hereof, and in which are shown by way of illustration embodiments that may be practiced. It is to be understood that other embodiments may be utilized and structural or logical changes may be made without departing from the scope. Therefore, the following detailed description is not to be taken in a limiting sense, and the scope of embodiments is defined by the appended claims and their equivalents.

Various operations may be described as multiple discrete operations in turn, in a manner that may be helpful in understanding embodiments; however, the order of description should not be construed to imply that these operations are order dependent.

For the purposes of the description, a phrase in the form “A/B” or in the form “A and/or B” means (A), (B), or (A and B). For the purposes of the description, a phrase in the form “at least one of A, B, and C” means (A), (B), (C), (A and B), (A and C), (B and C), or (A, B and C). For the purposes of the description, a phrase in the form “(A)B” means (B) or (AB) that is, A is an optional element.

The description may use the terms “embodiment” or “embodiments,” which may each refer to one or more of the same or different embodiments. Furthermore, the terms “comprising,” “including,” “having,” and the like, as used with respect to embodiments, are synonymous, and are generally intended as “open” terms (e.g., the term “including” should be interpreted as “including but not limited to,” the term “having” should be interpreted as “having at least,” the term “includes” should be interpreted as “includes but is not limited to,” etc.).

With respect to the use of any plural and/or singular terms herein, those having skill in the art can translate from the plural to the singular and/or from the singular to the plural as is appropriate to the context and/or application. The various singular/plural permutations may be expressly set forth herein for sake of clarity.

The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description.

Unless otherwise noted, technical terms are used according to conventional usage. Definitions of common terms in molecular biology can be found in Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); and Robert A. Meyers (ed.), Molecular Biology and Biotechnology: a Comprehensive Desk Reference, published by VCH Publishers, Inc., 1995 (ISBN 9780471185710); and other similar references.

Although methods and materials similar or equivalent to those described herein can be used in the practice or testing of this disclosure, suitable methods and materials are described below. For example, conventional methods well known in the art to which this disclosure pertains are described in various general and more specific references, including, for example, Sambrook et al., Molecular Cloning: A Laboratory Manual, 2d ed., Cold Spring Harbor Laboratory Press, 1989; Sambrook et al., Molecular Cloning: A Laboratory Manual, 3d ed., Cold Spring Harbor Press, 2001; Ausubel et al., Current Protocols in Molecular Biology, Greene Publishing Associates, 1992 (and Supplements to 2000); Ausubel et al., Short Protocols in Molecular Biology: A Compendium of Methods from Current Protocols in Molecular Biology, 4th ed., Wiley & Sons, 1999; Harlow and Lane, Antibodies: A Laboratory Manual, Cold Spring Harbor Laboratory Press, 1990; and Harlow and Lane, Using Antibodies: A Laboratory Manual, Cold Spring Harbor Laboratory Press, 1999. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

### I. Introduction

It has been recognized that frameshift neoantigens are important in determining the response to immunotherapeutics (IT), particularly checkpoint inhibitors (CPI) (anti-PD-1, -PDL-1, -CTLA4). The best predictor to date for the response to CPI therapy is microsatellite instability (MSI) status. Any tumor type that is MSI-high (MSI-H) has a 60-80% chance of significant clinical response. While colon, endometrial and stomach cancers have high incidence (12-25%) of MSI-H and are frequently screened (but not always) the remarkable responses of other MSI-H cancer types to CPI treatment has spurred efforts to screen all late stage cancers for MSI status. The current screens are invasive, qualitative, expensive, although blood based, NGS may relieve the requirement for biopsies. More importantly, cancers with low frequency of MSI are not routinely screened, denying the access to CPI as a primary treatment. Here, the inventors have developed a simple, non-invasive, antibody-based, serological assay to determine if a person has an MSI-H cancer. A description of the theory behind using FSP and their applications to vaccines are provided in Zhang et al. (Scientific Reports, 2018. 81:p 1-10) and Shen et al., (Scientific Reports, 2019). It has value in that it is simpler and probably more accurate than the state of the art, and its simplicity and low cost may be enough to merit screening many more patients for MSI status and allowing them to receive CPI treatment. The same diagnostic identifies FSPs that can comprise personal or off-the-shelf therapeutic vaccines for MSI-H patients.

The diagnostic assay for MSI is commonly either immunohistochemistry (IHC) or PCR from a tumor biopsy. IHC involves staining tumor sections for 4 proteins of the repair complex. Absence of one or more proteins in the cancer cells is scored as MSI-H. The PCR assay amplifies 6 large MSs. An indel in two or more MSs is scored as MSI-H, one as MSI-L and none as MSS (stable). MSI status can also be scored by next generation sequencing (NGS) of tumor DNA, but this is not commonly performed for reasons of cost and time. There is an interest in applying NGS for Tumor Mutational Burden to include MSI. However, this would depend on the wide acceptance of TMB as an assay and would still require a tumor biopsy. PCR and IHC tests have high concordance. Both the specificity and sensitivity are close to 100% for colon cancer, but the sensitivity is 80% for endometrial and prostate cancer.

Since the recognition that the high response rate to CPI treatment was due to MSI, colon cancer has been routinely screened. However, the remarkable response of individuals to CPI who had MSI-H even in cancers with low incidence of MSI, has led to the call for routine screening of late stage tumors. This could expand CPI treatment to individuals who many not normally have been urged to undergo CPI therapy.

Related to these developments is the realization of the importance of tumor generated frameshift (FS) peptide neoantigens in the immune response to the tumor and CPI treatment. MSI causes indels in MSs in coding regions. There are ˜8,000 MSs in coding regions. These indels would create FSPs downstream of the MSs. Response to CPI correlates with the number of FS mutations in tumors (10) better than total mutational burden or single nucleotide neoantigens (11, 12). This explains why renal cancer, with a low single nucleotide mutation burden but high FS mutation levels, responds well to CPI treatment. It is increasingly clear that FS are a key element in the immune response to CPI.

Even MSI status may be only indirectly correlated with relevant immune factors that determine anti-tumor activity. It is thought that MSI-H status creates a hypermutation state which more directly relates to CPI response. Other mutations such as POLE also creates hypermutation but is low in MSI indels. The ideal MSI diagnostic would directly measure the immune response to the most important neoantigens and be non-invasive.

Here, the inventors have developed a non-invasive, highly accurate serological assay to determine if a person has an MSI cancer. It has value in that it is simpler and as accurate as the state of the art, and its simplicity and low cost merit screening many more patients for MSI status and allowing them to receive CPI treatment. It is a fundamentally different method and system to screen for MSI status. It is based on a system the inventors created to manufacture peptide microarrays on which FSP, such as 100 to 400,000 (400K) FSP, could be produced downstream of microsatellites in coding sequences or from mis-splicing of exons. Instead of determining if a repair protein is missing (IHC) or there is an indel in a subset of MSs (PCR,NGS), the disclosed assay directly measures the immune response to FSPs produced in the tumor by using peptide microarrays composed of FSPs. As such, the disclosed assays are a more direct and simpler detecting MSI than IHC or PCR/NGS. That this discovery is the only one that directly measures the immune response, as opposed to changes in the DNA, it allows better resolution of who will respond to CPI treatment. Currently patients deemed MSI-Low are not provided CPI. However, the inventors have found that some of the MSI-L patients have significant immune reactivity and may respond to CPI. It also has been found that 40% of the MSI-H patients do not respond to CPI. Embodiments provided herein relate to methods that allow distinguishing these patients.

Peptide microarrays can be produced using an in situ synthetic method and offer a scalable platform in which several hundred identical peptide microarrays are produced on a silicon wafer. In some embodiments, the assay is an ELISA on which desired FSP are present, such as 100 to 400K FSP, to detect antibodies to them. In some examples, the disclosed assays includes indels in MSs as well as those which result from exon mis-splicing.

Besides being a more direct measure of what determines CPI response, the disclosed assay uses a biological sample, such as small amount of blood, rather than a tumor biopsy. Such a diagnostic is useful as 1) a replacement for the current standard assay for detecting and monitoring MSI-H cancer types, 2) as an extension of the MSI assay to routine screening of all late stage cancers; 3) an assay for cases where tumor tissue is not available (approximately 30%); and/or 4) allow screening of all cancers for MSI status, thereby greatly expanding the number of patients eligible for CPI therapy.

It is believed that the disclosed assay for MSI status can extend CPI treatment, such as to the approximately 5% of all cancer patients who are not currently screened but are MSI-H positive, and would greatly benefit from CPI treatment. This would include the approximately 30% of patients that cannot provide sufficient biopsy material. There are approximately 600,000 late stage cancers in the US/year and a $500 MSI screening assay would have a $300M/year market potential. If CPI treatment is extended to earlier stages of cancer, where MSI-H is more frequent, this diagnostic would have expanded impact, particularly as it is more difficult to get sufficient tumor tissue from smaller tumors.

### II. Terms

To facilitate review of the various embodiments of this disclosure, the following explanations of specific terms are provided, along with particular examples:

Adjuvant: A vehicle used to enhance antigenicity; such as a suspension of minerals (alum, aluminum hydroxide, aluminum phosphate) on which antigen is adsorbed; or water-in-oil emulsion in which antigen solution is emulsified in oil (MF-59, Freund's incomplete adjuvant), sometimes with the inclusion of killed mycobacteria (Freund's complete adjuvant) to further enhance antigenicity (inhibits degradation of antigen and/or causes influx of macrophages). Adjuvants also include immunostimulatory molecules, such as cytokines, costimulatory molecules, and for example, immunostimulatory DNA or RNA molecules, such as CpG oligonucleotides.

An adjuvant is a substance distinct from the antigen for which an immune response is desired. In several embodiments, an adjuvant enhances T cell activation by promoting the innate immune response leading to the accumulation and activation of other leukocytes (accessory cells) at the site of antigen exposure. Thus, adjuvants may enhance accessory cell expression of T cell-activating co-stimulators and cytokines and may also prolong the expression of peptide-MHC complexes on the surface of antigen-presenting cells.

Administration: The introduction of a composition or agent into a subject by a chosen route. Administration can be local or systemic. For example, if the chosen route is intravenous, the composition is administered by introducing the composition into a vein of the subject.

Agent: Any substance or any combination of substances that is useful for achieving an end or result; for example, a substance or combination of substances useful for inducing an immune response in a subject. Agents include peptides, (such as disclosed herein) proteins, nucleic acid molecules, compounds, small molecules, organic compounds, inorganic compounds, or other molecules of interest. An agent can include a therapeutic agent, a diagnostic agent or a pharmaceutical agent. In some embodiments, the agent is a polypeptide agent. The skilled artisan will understand that particular agents may be useful to achieve more than one result.

Antibody: Immunoglobulin molecules and immunologically active portions of immunoglobulin molecules, i.e., molecules that contain an antigen binding site that specifically binds (immunoreacts with) an antigen. A naturally occurring antibody (e.g., IgG, IgM, IgD) includes four polypeptide chains, two heavy (H) chains and two light (L) chains interconnected by disulfide bonds. However, it has been shown that the antigen-binding function of an antibody can be performed by fragments of a naturally occurring antibody. Thus, these antigen-binding fragments are also intended to be designated by the term “antibody.” Specific, non-limiting examples of binding fragments encompassed within the term antibody include (i) a Fab fragment consisting of the VL, VH, CL and CHi domains; (ii) an Fd fragment consisting of the VH and CHi domains; (iii) an Fv fragment consisting of the VL and VH domains of a single arm of an antibody, (iv) a dAb fragment (Ward et al., Nature 341:544-546, 1989) which consists of a VH domain; (v) an isolated complementarity determining region (CDR); and (vi) a F(ab′)2 fragment, a bivalent fragment comprising two Fab fragments linked by a disulfide bridge at the hinge region.

Immunoglobulins and certain variants thereof are known and many have been prepared in recombinant cell culture (e.g., see U.S. Pat. Nos. 4,745,055; 4,444,487; WO 88/03565; EP 256,654; EP 120,694; EP 125,023; Faoulkner et al., Nature 298:286, 1982; Morrison, J. Immunol. 123:793, 1979; Morrison et al., Ann Rev. Immunol 2:239, 1984 each of which is hereby incorporated by reference in its entirety). Humanized antibodies and fully human antibodies are also known in the art.

Antigen: A compound, composition, or substance that can stimulate the production of antibodies or a T cell response in an animal, including compositions that are injected or absorbed into an animal. An antigen reacts with the products of specific humoral or cellular immunity, including those induced by heterologous immunogens, such as the peptides disclosed herein. The term “antigen” includes all related antigenic epitopes. “Epitope” or “antigenic determinant” refers to a site on an antigen to which B and/or T cells respond. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 amino acids in a unique spatial conformation. Methods of determining spatial conformation of epitopes include, for example, x-ray crystallography and 2-dimensional nuclear magnetic resonance.

Animal: Living multi-cellular vertebrate organisms, a category that includes, for example, mammals and birds. The term mammal includes both human and non-human mammals. Similarly, the term “subject” includes both human and veterinary subjects, including dogs.

Array: An arrangement of molecules, such as biological macromolecules (such as peptides), in addressable locations on or in a substrate. A “microarray” is an array that is miniaturized so as to require or be aided by microscopic examination for evaluation or analysis. The array of molecules (“features”) makes it possible to carry out a very large number of analyses on a sample at one time. Within an array, each arrayed sample is addressable, in that its location can be reliably and consistently determined within at least two dimensions of the array. The feature application location on an array can assume different shapes. For example, the array can be regular (such as arranged in uniform rows and columns) or irregular. Thus, in ordered arrays, the location of each sample is assigned to the sample at the time when it is applied to the array, and a key may be provided in order to correlate each location with the appropriate target or feature position. Often, ordered arrays are arranged in a symmetrical grid pattern, but samples can be arranged in other patterns (such as in radially distributed lines, spiral lines, or ordered clusters). Addressable arrays usually are computer readable, in that a computer can be programmed to correlate a particular address on the array with information about the sample at that position (such as hybridization or binding data, including for instance signal intensity). In some examples of computer readable formats, the subject features in the array are arranged regularly, for instance in a Cartesian grid pattern, which can be correlated to address information by a computer.

Binding or stable binding: An association between two substances or molecules, such as the association of an antibody with a peptide. Binding can be detected by any procedure known to one skilled in the art, such as by physical or functional properties of the formed complexes, such as a target/antibody complex.

Clinical outcome: Refers to the health status of a patient following treatment for a disease or disorder, or in the absence of treatment. Clinical outcomes include, but are not limited to, an increase in the length of time until death, a decrease in the length of time until death, an increase in the chance of survival, an increase in the risk of death, survival, disease-free survival, chronic disease, metastasis, advanced or aggressive disease, disease recurrence, death, and favorable or poor response to therapy.

Computer readable media: Any medium or media, which can be read and accessed directly by a computer, so that the media is suitable for use in a computer system. Such media include, but are not limited to: magnetic storage media such as floppy discs, hard disc storage medium and magnetic tape; optical storage media such as optical discs or CD-ROM; electrical storage media such as RAM and ROM; and hybrids of these categories such as magnetic/optical storage media.

Computer system: Hardware that can be used to analyze atomic coordinate data and/or design an antigen using atomic coordinate data. The minimum hardware of a computer-based system typically comprises a central processing unit (CPU), an input device, for example a mouse, keyboard, and the like, an output device, and a data storage device. Desirably a monitor is provided to visualize structure data. The data storage device may be RAM or other means for accessing computer readable. Examples of such systems are microcomputer workstations available from Silicon Graphics Incorporated and Sun Microsystems running Unix based Windows NT or IBM OS/2 operating systems.

Contacting: Placement in direct physical association, including both solid or liquid forms.

Control: A sample or standard used for comparison with an experimental sample, such as a tumor sample obtained from a patient with a particular type of cancer. The control can be a sample obtained from a healthy patient or a non-tumor tissue sample obtained from a patient diagnosed with a particular type of cancer. A control can also be a historical control or standard reference value or range of values (i.e. a previously tested control sample, such as a group of cancer patients with poor prognosis, or group of samples that represent baseline or normal values).

A difference between a test sample and a control can be an increase or conversely a decrease. The difference can be a qualitative difference or a quantitative difference, for example a statistically significant difference. In some examples, a difference is an increase or decrease, relative to a control, of at least about 5%, such as at least about 10%, at least about 20%, at least about 30%, at least about 40%, at least about 50%, at least about 60%, at least about 70%, at least about 80%, at least about 90%, at least about 100%, at least about 150%, at least about 200%, at least about 250%, at least about³⁰⁰%, at least about 350%, at least about 400%, at least about 500%, or greater than 500%.

Diagnostic: Identifying the presence or nature of a pathologic condition. Diagnostic methods differ in their sensitivity and specificity. The “sensitivity” of a diagnostic assay is the percentage of diseased subjects who test positive (percent of true positives). The “specificity” of a diagnostic assay is 1 minus the false positive rate, where the false positive rate is defined as the proportion of those without the disease who test positive. While a particular diagnostic method may not provide a definitive diagnosis of a condition, it suffices if the method provides a positive indication that aids in diagnosis. “Prognostic” means predicting the probability of development (for example, severity) of a pathologic condition.

Effective amount: An amount of agent, such as an agent that is sufficient to generate a desired response, such an immune response. In some examples, an “effective amount” is one that treats (including prophylaxis) one or more symptoms and/or underlying causes of any of a disorder or disease, for example to treat and/or prevent cancer in a subject, such as a canine subject. In one example, an effective amount is a therapeutically effective amount. In one example, an effective amount is an amount that prevents one or more signs or symptoms of a particular disease or condition from developing, such as one or more signs or symptoms associated with cancer.

ELISA (enzyme linked immunosorbent assay): An assay for antibodies in sera, blood, etc. The assay uses a solid-phase enzyme immunoassay to detect the presence of a ligand in a liquid sample using antibodies directed against the protein to be measured. Peptides or proteins of interest are bound to a surface of a well, bead or other carrier and incubated with the sera to test. After binding, the well is washed and the amount of antibody bound to the test peptide/protein detected with a labeled anti-antibody secondary.

Exon Mis-splicing: In the context of this disclosure, a term meaning the joining of one exon of a gene with another of the same gene or different gene such that the frame of the resulting protein is shifted resulting in premature termination of the protein. This error in processing exons is greatly increased in tumors.

Expression: Translation of a nucleic acid into a peptide and/or protein. Peptides and/or proteins may be expressed and remain intracellular, become a component of the cell surface membrane, or be secreted into the extracellular matrix or medium.

Expression Control Sequences: Nucleic acid sequences that regulate the expression of a heterologous nucleic acid sequence to which it is operatively linked. Expression control sequences are operatively linked to a nucleic acid sequence when the expression control sequences control and regulate the transcription and, as appropriate, translation of the nucleic acid sequence. Thus, expression control sequences can include appropriate promoters, enhancers, transcription terminators, a start codon (ATG) in front of a protein-encoding gene, splicing signal for introns, maintenance of the correct reading frame of that gene to permit proper translation of mRNA, and stop codons. The term “control sequences” is intended to include, at a minimum, components whose presence can influence expression, and can also include additional components whose presence is advantageous, for example, leader sequences and fusion partner sequences. Expression control sequences can include a promoter. A promoter is a minimal sequence sufficient to direct transcription. Also included are those promoter elements which are sufficient to render promoter-dependent gene expression controllable for cell-type specific, tissue-specific, or inducible by external signals or agents; such elements may be located in the 5′ or 3′ regions of the gene. Both constitutive and inducible promoters are included (see for example, Bitter et al, Methods in Enzymology 153:516-544, 1987). A polynucleotide can be inserted into an expression vector that contains a promoter sequence, which facilitates the efficient transcription of the inserted genetic sequence.

Host cells: Cells in which a vector, such as a viral vector or DNA vector, can be propagated and its nucleic acid sequences expressed. The cell may be prokaryotic or eukaryotic. The term also includes any progeny of the subject host cell. It is understood that all progeny may not be identical to the parental cell since there may be mutations that occur during replication. However, such progeny are included when the term “host cell” is used.

Hybridization: Oligonucleotides and their analogs hybridize by hydrogen bonding, which includes Watson-Crick, Hoogsteen or reversed Hoogsteen hydrogen bonding, between complementary bases. Generally, nucleic acid consists of nitrogenous bases that are either pyrimidines (cytosine (C), uracil (U), and thymine (T)) or purines (adenine (A) and guanine (G)). These nitrogenous bases form hydrogen bonds between a pyrimidine and a purine, and the bonding of the pyrimidine to the purine is referred to as “base pairing.” More specifically, A will hydrogen bond to T or U, and G will bond to C. “Complementary” refers to the base pairing that occurs between two distinct nucleic acid sequences or two distinct regions of the same nucleic acid sequence. “Specifically hybridizable” and “specifically complementary” are terms that indicate a sufficient degree of complementarity such that stable and specific binding occurs between the oligonucleotide (or it is analog) and the DNA or RNA target. The oligonucleotide or oligonucleotide analog need not be 100% complementary to its target sequence to be specifically hybridizable. An oligonucleotide or analog is specifically hybridizable when binding of the oligonucleotide or analog to the target DNA or RNA molecule interferes with the normal function of the target DNA or RNA, and there is a sufficient degree of complementarity to avoid non-specific binding of the oligonucleotide or analog to non-target sequences under conditions where specific binding is desired. Such binding is referred to as specific hybridization.

Hybridization conditions resulting in particular degrees of stringency will vary depending upon the nature of the hybridization method of choice and the composition and length of the hybridizing nucleic acid sequences. Generally, the temperature of hybridization and the ionic strength (especially the Na+ concentration) of the hybridization buffer will determine the stringency of hybridization, though waste times also influence stringency. Calculations regarding hybridization conditions required for attaining particular degrees of stringency are discussed by Sambrook et al. (ed.), Molecular Cloning: A Laboratory Manual, 2nd ed., vol. 1-3, Cold Spring Harbor Laboratory Press, Cold Spring Harbor, N.Y., 1989, chapters 9 and 11.

Immune checkpoint inhibitor therapy (CPI or ICI): A form of cancer immunotherapy. The therapy targets immune checkpoints, key regulators of the immune system that when stimulated can dampen the immune response to an immunologic stimulus. Some cancers can protect themselves from attack by stimulating immune checkpoint targets. Checkpoint therapy can block inhibitory checkpoints, restoring immune system function.

Currently approved checkpoint inhibitors target the molecules CTLA4, PD-1, and PD-L1. PD-1 is the transmembrane programmed cell death 1 protein (also called PDCD1 and CD279), which interacts with PD-L1 (PD-1 ligand 1, or CD274). PD-L1 on the cell surface binds to PDi on an immune cell surface, which inhibits immune cell activity. Among PD-L1 functions is a key regulatory role on T cell activities. It appears that (cancer-mediated) upregulation of PD-L1 on the cell surface may inhibit T cells that might otherwise attack. Antibodies that bind to either PD-1 or PD-L1 and therefore block the interaction may allow the T-cells to attack the tumor.

Immune checkpoint inhibitors, such as anti-PD-1 antibodies, have been approved to treat different types of cancer (e.g., bladder, lung, kidney, melanoma, head, neck, Hodgkin's lymphoma and solid tumors). PD-1 inhibitors include nivolumab, pembrolizumab, cemiplimab and spartalizumab. Additional CPIs include CTLA-4 blockage (e.g., ipilimumab, such as for treatment of melanoma) and PD-LI inhibitors (e.g., atezolizumab, avelumab, or durvalumab, such as for treatment of bladder cancer). Remarkably, the FDA for the first time gave tumor-type, agnostic approval to treat any late stage cancer that is MSI-H. This was based on the remarkably positive responses to treatment of not only cancers with frequent MSI-H phenotypes (colon, endometrial and stomach), but rare MSI-H patients in other cancers. For example, a woman with triple negative, metastatic breast cancer who was MSI-H had a complete remission, while most breast cancers have been unresponsive to CPI treatment.

Immune response: A response of a cell of the immune system, such as a B cell, T cell, or monocyte, to a stimulus. In one embodiment, the response is specific for a particular antigen (an “antigen-specific response”).

Immunogenic peptide: A peptide which comprises an allele-specific motif or other sequence such that the peptide will bind an MHC molecule and induce a cytotoxic T lymphocyte (“CTL”) response, or a B cell response (e.g., antibody production) against the antigen from which the immunogenic peptide is derived.

Immunogenic composition: A composition that includes an immunogenic polypeptide or nucleic acid or viral vector encoding an immunogenic polypeptide that induces a measurable immune response (such as a CTL response or measurable B cell response) against the immunogenic polypeptide. For example, in several embodiments, an immunogenic composition includes a viral vector expressing an immunogenic polypeptide that induces an immune response to an epitope on the immunogenic polypeptide that is also contained on a polypeptide expressed by a viral pathogen. In one example, an immunogenic composition includes a nucleic acid encoding an immunogenic polypeptide, such as a nucleic acid vector that can be used to express the polypeptide (and thus be used to elicit an immune response against this polypeptide or an epitope on the polypeptide). In several examples, the immunogenic composition includes one or more adjuvants.

Immunotherapy: A method of evoking an immune response against a virus based on its production of target antigens. Immunotherapy based on cell-mediated immune responses involves generating a cell-mediated response to cells that produce particular antigenic determinants, while immunotherapy based on humoral immune responses involves generating specific antibodies to virus that produce particular antigenic determinants. In several embodiments, immunotherapy includes administration of prime-boost vaccination to a subject.

Inhibiting or treating a disease: Inhibiting the full development of a disease or condition, for example cancer. “Treatment” refers to a therapeutic intervention that ameliorates a sign or symptom of a disease or pathological condition after it has begun to develop. The term “ameliorating,” with reference to a disease or pathological condition, refers to any observable beneficial effect of the treatment. Inhibiting a disease can include preventing or reducing the risk of the disease. The beneficial effect can be evidenced, for example, by a delayed onset of clinical symptoms of the disease in a susceptible subject, a reduction in severity of some or all clinical symptoms of the disease, a slower progression of the disease, an improvement in the overall health or well-being of the subject, or by other parameters well known in the art that are specific to the particular disease. A “prophylactic” treatment is a treatment administered to a subject who does not exhibit signs of a disease or exhibits only early signs for the purpose of decreasing the risk of developing pathology.

Isolated: An “isolated” biological component (such as a nucleic acid molecule, peptide, protein, or organelle) has been substantially separated or purified away from other biological components in the cell of the organism in which the component naturally occurs, e.g., other chromosomal and extra-chromosomal DNA and RNA, proteins and/organelles. Nucleic acids and proteins that have been “isolated” include nucleic acids and proteins purified by standard purification methods. The term also embraces nucleic acids and proteins prepared by recombinant expression in a host cell as well as chemically synthesized nucleic acids, such as probes and primers.

Label: A detectable compound or composition that is conjugated directly or indirectly to another molecule to facilitate detection of that molecule. Specific, non-limiting examples of labels include fluorescent tags, enzymatic linkages, and radioactive isotopes.

Microsatellite: A tract of repetitive DNA/RNA in which certain DNA/RNA motifs are repeated, typically 5-50 times. Microsatellites occur at thousands of locations within an organism's genome. They have a higher mutation rate than other areas of DNA/RNA leading to high genetic diversity. MSI is defined by the frequent insertion or deletion (indel) of a base in a microsatellite (MS). This is caused by a failure in mis-match repair (dMMR). MSs are prone to indels because of the loss of register in repeats, particularly runs of A/T, during replication. The most frequent defects causing dMMR are methylation or mutations in hMLH1, hPMS1-8, hPMSR1-7, hMSH2 and hMSH3 in the tumor or in the germline (Lynch syndrome). The frequency of MSI-H in cancers is variable. Colon (16.6%), endometrial (28.3%) and stomach (21.9%) have the highest frequency, but small numbers of MSI-H patients (0.1-9%) have been detected in 22 other cancers and probably occur in all cancers at some frequency (Cortes-Ciriano I, Lee S, Park W Y, Kim T M, Park P J. A molecular portrait of microsatellite instability across multiple cancers. Nat Commun. 2017; 8:15180. doi: 10.1038/ncomms15180. PubMed PMID: 28585546; PMCID: PMC5467167., which is hereby incorporated by reference). Early cancer stages have higher MSI-H frequencies than later (Shannon C, Kirk J, Barnetson R, Evans J, Schnitzler M, Quinn M, Hacker N, Crandon A, Harnett P. Incidence of microsatellite instability in synchronous tumors of the ovary and endometrium. Clin Cancer Res. 2003; 9(4):1387-92. PubMed PMID: 12684409, which is hereby incorporated by reference in its entirety).

Nucleic acid: A polymer composed of nucleotide units (ribonucleotides, deoxyribonucleotides, related naturally occurring structural variants, and synthetic non-naturally occurring analogs thereof) linked via phosphodiester bonds, related naturally occurring structural variants, and synthetic non-naturally occurring analogs thereof. Thus, the term includes nucleotide polymers in which the nucleotides and the linkages between them include non-naturally occurring synthetic analogs, such as, for example and without limitation, phosphorothioates, phosphoramidates, methyl phosphonates, chiral-methyl phosphonates, 2′-O-methyl ribonucleotides, peptide-nucleic acids (PNAs), and the like. Such polynucleotides can be synthesized, for example, using an automated DNA synthesizer. The term “oligonucleotide” typically refers to short polynucleotides, generally no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

Conventional notation is used herein to describe nucleotide sequences: the left-hand end of a single-stranded nucleotide sequence is the 5′-end; the left-hand direction of a double-stranded nucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand;” sequences on the DNA strand having the same sequence as an mRNA transcribed from that DNA and which are located 5′ to the 5′-end of the RNA transcript are referred to as “upstream sequences;” sequences on the DNA strand having the same sequence as the RNA and which are 3′ to the 3′ end of the coding RNA transcript are referred to as “downstream sequences.”

“cDNA” refers to a DNA that is complementary or identical to an mRNA, in either single stranded or double stranded form.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA produced by that gene produces the protein in a cell or other biological system. Both the coding strand, the nucleotide sequence of which is identical to the mRNA sequence and is usually provided in sequence listings, and non-coding strand, used as the template for transcription, of a gene or cDNA can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

“Recombinant nucleic acid” refers to a nucleic acid having nucleotide sequences that are not naturally joined together and can be made by artificially combining two otherwise separated segments of sequence. This artificial combination is often accomplished by chemical synthesis or, more commonly, by the artificial manipulation of isolated segments of nucleic acids, for example, by genetic engineering techniques. Recombinant nucleic acids include nucleic acid vectors comprising an amplified or assembled nucleic acid which can be used to transform a suitable host cell. A host cell that comprises the recombinant nucleic acid is referred to as a “recombinant host cell.” The gene is then expressed in the recombinant host cell to produce a “recombinant polypeptide.” A recombinant nucleic acid can also serve a non-coding function (for example, promoter, origin of replication, ribosome-binding site and the like).

Nucleotide: “Nucleotide” includes, but is not limited to, a monomer that includes a base linked to a sugar, such as a pyrimidine, purine or synthetic analogs thereof, or a base linked to an amino acid, as in a peptide nucleic acid (PNA). A nucleotide is one monomer in a polynucleotide. A nucleotide sequence refers to the sequence of bases in a polynucleotide.

Operably linked: A first nucleic acid sequence is operably linked with a second nucleic acid sequence when the first nucleic acid sequence is placed in a functional relationship with the second nucleic acid sequence. For instance, a promoter, such as the CMV promoter, is operably linked to a coding sequence if the promoter affects the transcription or expression of the coding sequence. Generally, operably linked DNA sequences are contiguous and, where necessary to join two protein-coding regions, in the same reading frame.

Peptide: Any chain of amino acids, regardless of length or post-translational modification (e.g., glycosylation or phosphorylation). A polypeptide can be between 3 and 30 amino acids in length. In one embodiment, a polypeptide is from about 5 to about 25 amino acids in length. In yet another embodiment, a polypeptide is from about 8 to about 12 amino acids in length. In yet another embodiment, a peptide is about 5 amino acids in length. With regard to polypeptides, the word “about” indicates integer amounts.

Peptide Modifications: Immunogenic peptides include synthetic embodiments of peptides described herein. In addition, analogs (non-peptide organic molecules), derivatives (chemically functionalized peptide molecules obtained starting with the disclosed peptide sequences) and variants (homologs) of these proteins can be utilized in the methods described herein. Each polypeptide of this disclosure is comprised of a sequence of amino acids, which may be either L and/or D-amino acids, naturally occurring and otherwise.

Peptides can be modified by a variety of chemical techniques to produce derivatives having essentially the same activity as the unmodified peptides, and optionally having other desirable properties. For example, carboxylic acid groups of the protein, whether carboxyl-terminal or side chain, can be provided in the form of a salt of a pharmaceutically-acceptable cation or esterified to form a C1-C16 ester, or converted to an amide of formula NR1R2 wherein R1 and R2 are each independently H or C1-C16 alkyl, or combined to form a heterocyclic ring, such as a 5- or 6-membered ring. Amino groups of the peptide, whether amino-terminal or side chain, can be in the form of a pharmaceutically-acceptable acid addition salt, such as the HCl, HBr, acetic, benzoic, toluene sulfonic, maleic, tartaric and other organic salts, or can be modified to C1-C16 alkyl or dialkyl amino or further converted to an amide.

Hydroxyl groups of the peptide side chains may be converted to C1-C16 alkoxy or to a C1-C16 ester using well-recognized techniques. Phenyl and phenolic rings of the peptide side chains may be substituted with one or more halogen atoms, such as fluorine, chlorine, bromine or iodine, or with C1-C16 alkyl, C1-C16 alkoxy, carboxylic acids and esters thereof, or amides of such carboxylic acids. Methylene groups of the peptide side chains can be extended to homologous C2-C4 alkylenes. Thiols can be protected with any one of a number of well-recognized protecting groups, such as acetamide groups. Those skilled in the art will also recognize methods for introducing cyclic structures into the peptides of this disclosure to select and provide conformational constraints to the structure that result in enhanced stability.

Peptidomimetic and organomimetic embodiments are envisioned, whereby the three-dimensional arrangement of the chemical constituents of such peptido- and organomimetics mimic the three-dimensional arrangement of the peptide backbone and component amino acid side chains, resulting in such peptido- and organomimetics of an immunogenic polypeptide having measurable or enhanced ability to generate an immune response. For computer modeling applications, a pharmacophore is an idealized three-dimensional definition of the structural requirements for biological activity. Peptido- and organomimetics can be designed to fit each pharmacophore with current computer modeling software (using computer assisted drug design or CADD). See Walters, “Computer-Assisted Modeling of Drugs,” in Klegerman & Groves, eds., 1993, Pharmaceutical Biotechnology, Interpharm Press: Buffalo Grove, Ill., pp. 165 174 and Principles of Pharmacology, Munson (ed.) 1995, Ch. 102, for descriptions of techniques used in CADD. Also included are mimetics prepared using such techniques.

Pharmaceutical agent: A chemical compound or composition capable of inducing a desired therapeutic or prophylactic effect when properly administered to a subject or a cell. In some examples, a pharmaceutical agent includes one or more of the disclosed polypeptides.

Pharmaceutically acceptable carriers: The pharmaceutically acceptable carriers of use are conventional. Remington's Pharmaceutical Sciences, by E. W. Martin, Mack Publishing Co., Easton, Pa., 19th Edition, 1995, describes compositions and formulations suitable for pharmaceutical delivery of the compositions disclosed herein.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (such as powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Prime-boost vaccination: An immunotherapy including administration of a first immunogenic composition (the primer vaccine) followed by administration of a second immunogenic composition (the booster vaccine) to a subject to induce an immune response.

The booster vaccine is administered to the subject after the primer vaccine; the skilled artisan will understand a suitable time interval between administration of the primer vaccine and the booster vaccine, and examples of such timeframes are disclosed herein.

In some embodiments, the primer vaccine, the booster vaccine, or both primer vaccine and the booster vaccine additionally include an adjuvant.

Purified: The term purified does not require absolute purity; rather, it is intended as a relative term. Thus, for example, a purified polypeptide preparation is one in which the peptide or protein is more enriched than the peptide or protein is in its natural environment within a cell. In one embodiment, a preparation is purified such that the protein or peptide represents at least 50% of the total peptide or protein content of the preparation.

Recombinant: A recombinant nucleic acid is one that has a sequence that is not naturally occurring or has a sequence that is made by an artificial combination of two otherwise separated segments of sequence. This artificial combination is often accomplished by chemical synthesis or, more commonly, by the artificial manipulation of isolated segments of nucleic acids, e.g., by genetic engineering techniques.

Sample: As used herein, a “sample” obtained from a subject refers to a cell, fluid or tissue sample. Bodily fluids include, but are not limited to, blood, serum, urine, saliva and spinal fluid. Cell samples include, for example, PBMCs, white blood cells, lymphocytes, or other cells of the immune system.

Sequence identity: The similarity between amino acid sequences is expressed in terms of the similarity between the sequences, otherwise referred to as sequence identity. Sequence identity is frequently measured in terms of percentage identity (or similarity or homology); the higher the percentage, the more similar the two sequences are. Homologs or variants of a polypeptide will possess a relatively high degree of sequence identity when aligned using standard methods.

Within the context of an immunogenic peptide, a “conserved residue” is one which appears in a significantly higher frequency than would be expected by random distribution at a particular position in a peptide. In one embodiment, a conserved residue is one where the MHC structure may provide a contact point with the immunogenic peptide.

Methods of alignment of sequences for comparison are well known in the art. Various programs and alignment algorithms are described in: Smith and Waterman, Adv. Appl. Math. 2:482, 1981; Needleman and Wunsch, J. Mol. Biol. 48:443, 1970; Higgins and Sharp, Gene 73:237, 1988; Higgins and Sharp, CABIOS 5:151, 1989; Corpet et al., Nucleic Acids Research 16:10881, 1988; and Pearson and Lipman, Proc. Natl. Acad. Sci. USA 85:2444, 1988. Altschul et al., Nature Genet. 6:119, 1994, presents a detailed consideration of sequence alignment methods and homology calculations.

The NCBI Basic Local Alignment Search Tool (BLAST) (Altschul et al., J. Mol. Biol. 215:403, 1990) is available from several sources, including the National Center for Biotechnology Information (NCBI, Bethesda, Md.) and on the internet, for use in connection with the sequence analysis programs blastp, blastn, blastx, tblastn and tblastx. A description of how to determine sequence identity using this program is available on the NCBI website on the internet.

Homologs and variants of a polypeptide are typically characterized by possession of at least 75%, for example at least 80%, sequence identity counted over the full length alignment with the amino acid sequence using the NCBI Blast 2.0, gapped blastp set to default parameters. For comparisons of amino acid sequences of greater than about 30 amino acids, the Blast 2 sequences function is employed using the default BLOSUM62 matrix set to default parameters, (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties). Proteins with even greater similarity to the reference sequences will show increasing percentage identities when assessed by this method, such as at least 80%, at least 85%, at least 90%, at least 95%, at least 98%, or at least 99% sequence identity. Methods for determining sequence identity over such short windows are available at the NCBI website on the internet. One of skill in the art will appreciate that these sequence identity ranges are provided for guidance only; it is entirely possible that strongly significant homologs could be obtained that fall outside of the ranges provided.

Suitable methods and materials for the practice or testing of this disclosure are described below. Such methods and materials are illustrative only and are not intended to be limiting. Other methods and materials similar or equivalent to those described herein can be used. For example, methods well known in the art to which this disclosure pertains are described in various general and more specific references, including, for example, Sambrook et al., Molecular Cloning: A Laboratory Manual, 2d ed., Cold Spring Harbor Laboratory Press, 1989; Sambrook et al., Molecular Cloning: A Laboratory Manual, 3d ed., Cold Spring Harbor Press, 2001; Ausubel et al., Current Protocols in Molecular Biology, Greene Publishing Associates, 1992 (and Supplements to 2000); Ausubel et al., Short Protocols in Molecular Biology: A Compendium of Methods from Current Protocols in Molecular Biology, 4th ed., Wiley & Sons, 1999; Harlow and Lane, Antibodies: A Laboratory Manual, Cold Spring Harbor Laboratory Press, 1990; and Harlow and Lane, Using Antibodies: A Laboratory Manual, Cold Spring Harbor Laboratory Press, 1999. In addition, the materials, methods, and examples are illustrative only and not intended to be limiting.

Treatment: A method of reducing the effects of a disease or condition. Treatment can also refer to a method of reducing the disease or condition itself rather than just the symptoms. The treatment can be any reduction from native levels and can be but is not limited to the complete ablation of the disease, condition, or the symptoms of the disease or condition. For example, a disclosed method for reducing the effects of a cancer is considered to be a treatment if there is a 10% reduction in one or more symptoms of the disease (e.g., tumor size) in a subject with the disease when compared to native levels in the same subject or control subjects. Thus, the reduction can be a 10, 20, 30, 40, 50, 60, 70, 80, 90, 100%, or any amount of reduction in between as compared to native or control levels. It is also understood and contemplated herein that treatment can refer to any reduction in the progression of a disease or cancer. Thus, for example, methods of reducing the effects of a cancer is considered to be a treatment if there is a 10% reduction in the tumor growth rate relative to a control subject or tumor growth rates in the same subject prior to the treatment. It is understood that the reduction can be a 10, 20, 30, 40, 50, 60, 70, 80, 90, 100%, or any amount of reduction in between as compared to native or control levels.

Tumor: All neoplastic cell growth and proliferation, whether malignant or benign, and all pre-cancerous and cancerous cells and tissues.

“Solid tumor” is an abnormal mass of tissue that usually does not contain cysts or liquid areas. Solid tumors can be either benign (not cancer), or malignant (cancer). Different types of solid tumors are named for the type of cells that form them. Examples of solid tumors include, but are not limited to, sarcomas, carcinomas and lymphomas.

Vaccine: A preparation of immunogenic material capable of stimulating an immune response, administered for the prevention, amelioration, or treatment of infectious or other types of disease, such as cancer. The immunogenic material may include antigenic proteins, peptides or DNA derived from them.

The immunogenic material for a cancer vaccine may include, for example, a protein or peptide expressed by a tumor or cancer cell. Vaccines may elicit both prophylactic (preventative) and therapeutic responses. Methods of administration vary according to the vaccine, but may include inoculation, ingestion, inhalation or other forms of administration.

Vector: A nucleic acid molecule as introduced into a host cell, thereby producing a transformed host cell. A vector may include nucleic acid sequences that permit it to replicate in a host cell, such as an origin of replication. A vector may also include one or more selectable marker genes and other genetic elements known in the art. Recombinant DNA vectors are vectors having recombinant DNA.

Recombinant RNA vectors are vectors having recombinant RNA. A vector can include nucleic acid sequences that permit it to replicate in a host cell, such as an origin of replication. A vector can also include one or more selectable marker genes and other genetic elements known in the art. Viral vectors are recombinant vectors having at least some nucleic acid sequences derived from one or more viruses.

### III. System and Method

i. Overview

Disclosed herein are systems and methods for determining MSI status. In particular, the inventors have developed a non-invasive MSI test using high-density FS peptide arrays to evaluate the MSI status by measuring the antibody responses to the potential FS antigens. The disclosed assay is based on the logic that MSI-H patients produce FSP downstream of MS and that MS instability leads to more exon mis-splicing, creating more FSP. It is the formation of FSP that creates the favorable clinical response to CPI treatment in MSI-H patients and what the disclosed assay directly measures. The diagnosis of MSI-H status also indicates which FSP are reactive in MSI-H patients, enabling constitution of a vaccine to treat MSI-H cancers.

The homopolymer in human genome is the most susceptible MS region of insertion and/or deletion (INDEL) caused by the MSI/MMIR. There are total ˜7,000 homopolymers (longer than 7 nt repeats) regions in the coding regions of all human genes. The FS mutation by INDELs in these MS region can potentially generate ˜14K possible FSP. The MSI-H tumor also has more FS mis-splicing and exon 1 mis-initiation. There are 200 K FSPs that could be produced by exon mis-splicing and exon 1 mis-initiation. All potential FS peptides longer than 15 aa were divided into 15 aa peptides and in situ synthesized on a peptides array.

The higher MSI, the higher expression of more FS peptides in the tumor and the higher the immune response, including antibody immune responses to these FSPs in the patient. With this peptide array, one can comprehensively evaluate the overall MSI status of a tumor and the activity of the anti-tumor immune responses to all potential FS antigens, which is directly related to the level of the response of the cancer patient to the immunotherapy.

This technology has numerous applications. First, it can greatly simplify the assays that are currently being done routinely for MSI-H cancers. Instead of obtaining a biopsy, such as a biopsy of the colon, endometrial or stomach cancer and sequencing only 6 MS as representative of the status, one would apply approximately 10 μl of diluted blood to the FSP array and from the antibody binding, make a diagnostic assessment. The disclosed methods and systems are faster, less expensive and more informative than the conventional technology. Second, for many types of cancer the incidence of MSI is very low, 0.5-9%, with most having 1-2% incidence. Because of low incidence and cost, these cancer patients are not routinely screened for MSI status and may not receive checkpoint inhibitors. Given the simplicity and low cost of the disclosed FSP array assay, these patients could be screened for MSI status. This may allow more of these patients to receive checkpoint inhibitor treatment or earlier treatment. Even though this would require many more assays than for high MSI tumors, it would be a net benefit for treating patients. In addition, for patients receiving CPI, the arrays could be used to monitor response to the therapy and predict outcomes. The expectation is that if the tumor is being eradicated the antibody level to its FSP will decline. Patients with such a profile would be expected to have better outcomes.

In some embodiments, the disclosed methods and systems are used for determining whether a cancer patient has high microsatellite instability (MSI-H), or normal microsatellite stability. In some cases, it may be useful to determine the MSI-L cases as therapy recommendations evolve. The disclosed methods and systems can be used for identifying MSI-H and thus, patients with MSI-H tumors and candidates for immunotherapy, such as CPI therapy. In turn, the disclosed methods and systems can be used to predict responsiveness, based on being MSI-H, to immunotherapy, such as CPI therapy. In some embodiments, the methods further include providing an immunotherapy, such as CPI therapy, to the MSI-H patient, such as to a patient with colon cancer, endometrial cancer, melanoma, lung cancer, neck cancer, kidney cancer, bladder cancer, head cancer, stomach cancer, Hodgkin's lymphoma and/or solid tumors. In some embodiments, the disclosed methods and systems allow the immune response to components of FSP vaccine to be monitored. For example, one type of cancer vaccine uses the FSP as components. This can be a therapeutic or prophylactic vaccine. The immune response subsequent to vaccination with FSPs can be directly assessed on the arrays.

In some embodiments, the biological sample comprises or is selected from the group consisting of blood, plasma, serum, thymus, bone marrow, spleen, lymph node, bronchoalveolar lavage, breast, central nervous system, cerebrospinal fluid, eye, tears, gastrointestinal tract, saliva, feces, urine, heart, kidney, liver, lung, muscle, pancreas, peripheral nervous system, saliva, skin, thyroid, trachea, and tumor. In some embodiments, the biological sample is blood, serum, plasma, or saliva. In some embodiments, the biological sample comprises cells selected from B cells, T cells, CD4+ T cells, CD8+ T cells, Th17 cells, and combinations thereof. In some embodiments, the biological sample comprises an antibody. In some embodiments, all the biological samples tested are the same type. In other embodiments, the biological samples are different types, such as different types of samples listed above. In some examples, the biological sample is a fluid sample including antibody, such as blood, saliva or plasma.

In some embodiments, the systems disclosed herein includes a photolithographic array synthesis platform that merges semiconductor manufacturing processes and combinatorial chemical synthesis to produce array-based libraries on silicon wafers. By utilizing the tremendous advancements in photolithographic feature patterning, the array synthesis platform is highly-scalable and capable of producing combinatorial peptide libraries with 80 million features on an 8-inch wafer. Photolithographic array synthesis is performed using semiconductor wafer production equipment in a cleanroom to achieve high reproducibility. When the wafer is diced into standard microscope slide dimensions, each slide contains more than 6 million distinct chemical entities.

In some embodiments, arrays with peptide libraries produced by photolithographic technologies disclosed herein are used for immune-based assays. In some embodiments, platforms disclosed herein comprise frameshift peptides, such as peptides resulting from an insertion or deletion error in transcription of an mRNA or peptides resulting from a splicing error such as a trans-splicing error or a cis-splicing error. In some embodiments, platforms herein comprise frameshift peptides comprising peptides having a sequence selected from all MS FS or MS FS from oncogenes, essential genes, and/or highly expressed genes.

In some embodiments, the array is a wafer-based, photolithographic, in situ peptide array produced using reusable masks and automation to obtain arrays of scalable numbers of combinatorial sequence peptides. In some embodiments, the peptide array comprises about 20, about 50, about 70, about 100, about 500, about 1000, about 2000, about 3000, about 4000, about 5,000, about 6000, about 7000, about 8000, about 9000, about 10,000, about 15,000, about 20,000, about 30,000, about 40,000, about 50,000, about 100,000, about 200,000, about 300,000, about 400,000, about 500,000, or more peptides having different sequences. Multiple copies of each of the different sequence peptides can be situated on the wafer at addressable locations known as features. In some embodiments, the array comprises, consists essentially of, or consists of one or more of the peptides with sequences indicative of MSI, such as those set forth in any one of Tables 1-3. For example, Tables 1 and 2 provide frameshift peptides chosen from the 400K FSPs based on their ability to classify MSI-H from microsatellite stable (MSS) patients. These peptides also can constitute a vaccine for MSI-H patients.

TABLE 1

| | | peptide_ | SEQ ID | | SEQ ID | |
|---|---|---|---|---|---|---|
| nimb_id | desc_id | sequence | NO: | frameshift | NO: | fsid |
| HCIM011507 | CCDS50 | PQVGK | 1 | PQVGKKLWQLCWI | 81 | 235570 |
| | 91.2_ | KLWQL | | MTCLRLFTRNHFAV | | |
| | del_1 | CWIMT | | ILLMYQKSPGRLVD | | |
| | | | | GSSW | | |
| HCIM011508 | CCDS50 | CLRLFT | 2 | PQVGKKLWQLCWI | 81 | 235570 |
| | 91.2_ | RNHFA | | MTCLRLFTRNHFAV | | |
| | del_2 | VILL | | ILLMYQKSPGRLVD | | |
| | | | | GSSW | | |
| HCIMO11510 | CCDS50 | MYQKS | 3 | PQVGKKLWQLCWI | 81 | 235570 |
| | 91.2_ | PGRLV | | MTCLRLFTRNHFAV | | |
| | del_3 | DGSSW | | ILLMYQKSPGRLVD | | |
| | | | | GSSW | | |
| HCIM030952 | NM_000 | HEAGL | 4 | HEAGLGLHLEGTLW | 82 | 181308 |
| | 695.3_Ex | GLHLE | | PGPHHRTLELPTEPD | | |
| | on4_3rd_ | GTLWP | | PGAPGGRPRR | | |
| | 1 | | | | | |
| HCIM030953 | NM_000 | GPHHR | 5 | HEAGLGLHLEGTLW | 82 | 181308 |
| | 695.3_Ex | TLELPT | | PGPHHRTLELPTEPD | | |
| | on4_3rd_ | EPDP | | PGAPGGRPRR | | |
| | 2 | | | | | |
| HCIM033690 | NM 000 | PSTGCP | 6 | PSTGCPPPGKALLQG | 83 | 210409 |
| | 836.2_Ex | PPGKAL | | FLHRHSEAAGAYHR | | |
| | on6_3rd_ | LQG | | LQLRPLPGHQWQAR | | |
| | 1 | | | KEDRWRLERHDRG | | |
| HCIM033691 | NM_000 | FLHRHS | 7 | PSTGCPPPGKALLQG | 83 | 210409 |
| | 836.2_Ex | EAAGA | | FLHRHSEAAGAYHR | | |
| | on6_3rd_ | YHRL | | LQLRPLPGHQWQAR | | |
| | 2 | | | KEDRWRLERHDRG | | |
| HCIM033692 | NM 000 | QLRPLP | 8 | PSTGCPPPGKALLQG | 83 | 210409 |
| | 836.2_Ex | GHQWQ | | FLHRHSEAAGAYHR | | |
| | on6_3rd_ | ARKE | | LQLRPLPGHQWQAR | | |
| | 3 | | | KEDRWRLERHDRG | | |
| HCIM041740 | NM 001 | GRATL | 9 | GRATLQRGGFGAGA | 84 | 145701 |
| | 005291.2_ | QRGGF | | GRAVRSGRGAADR | | |
| | Exon1_ | GAGAG | | HR | | |
| | 3rd_1 | | | | | |
| HCIM067872 | NM_001 | VSRHV | 10 | VSRHVALGLPHLGS | 85 | 152624 |
| | 048218.1 | ALGLP | | LQWAPTSGSSPTQP | | |
| | Exon5_ | HLGSL | | WE | | |
| | 3rd_1 | | | | | |
| HCIM067873 | NM_001 | QWAPT | 11 | VSRHVALGLPHLGS | 85 | 152624 |
| | 048218.1 | SGSSPT | | LQWAPTSGSSPTQP | | |
| | Exon5_ | QPWE | | WE | | |
| | 3rd_2 | | | | | |
| HCIM071574 | NM 001 | EEPLD | 12 | EEPLDWSQSLSQTH | 86 | 221751 |
| | 080414.3 | WSQSL | | QTKERGFEGTLKIHR | | |
| | Exon25_ | SQTHQ | | GQPSLAAGVLRPRL | | |
| | 3rd_1 | | | AGGLSAAQITGREP | | |
| | | | | RHPRTGLQLCRRAR | | |
| | | | | RPQRVCGE | | |
| HCIM071575 | NM_001 | TKERGF | 13 | EEPLDWSQSLSQTH | 86 | 221751 |
| | 080414.3 | EGTLKI | | QTKERGFEGTLKIHR | | |
| | Exon25_ | HRG | | GQPSLAAGVLRPRL | | |
| | 3rd_2 | | | AGGLSAAQITGREP | | |
| | | | | RHPRTGLQLCRRAR | | |
| | | | | RPQRVCGE | | |
| HCIM071576 | NM_001 | QPSLAA | 14 | EEPLDWSQSLSQTH | 86 | 221751 |
| | 080414.3 | GVLRP | | QTKERGFEGTLKIHR | | |
| | Exon25_ | RLAG | | GQPSLAAGVLRPRL | | |
| | 3rd_3 | | | AGGLSAAQITGREP | | |
| | | | | RHPRTGLQLCRRAR | | |
| | | | | RPQRVCGE | | |
| HCIM071577 | NM_001 | GLSAA | 15 | EEPLDWSQSLSQTH | 86 | 221751 |
| | 080414.3 | QITGRE | | QTKERGFEGTLKIHR | | |
| | Exon25_ | PRHP | | GQPSLAAGVLRPRL | | |
| | 3rd_4 | | | AGGLSAAQITGREP | | |
| | | | | RHPRTGLQLCRRAR | | |
| | | | | RPQRVCGE | | |
| HCIM071578 | NM_001 | RTGLQ | 16 | EEPLDWSQSLSQTH | 86 | 221751 |
| | 080414.3 | LCRRA | | QTKERGFEGTLKIHR | | |
| | Exon25_ | RRPQR | | GQPSLAAGVLRPRL | | |
| | 3rd_5 | | | AGGLSAAQITGREP | | |
| | | | | RHPRTGLQLCRRAR | | |
| | | | | RPQRVCGE | | |
| HCIM081794 | NM_001 | VVCQE | 17 | VVCQEGWSGPLLAQ | 87 | 182759 |
| | 100876.1 | GWSGP | | RLVPASLGQSQPGIT | | |
| | Exon 11 | LLAQR | | ASLCPPQCRE | | |
| | 2nd 1 | | | | | |
| HCIM081795 | NM_001 | LVPASL | 18 | VVCQEGWSGPLLAQ | 87 | 182759 |
| | 100876.1 | GQSQP | | RLVPASLGQSQPGIT | | |
| | Exon11_ | GITA | | ASLCPPQCRE | | |
| | 2nd_2 | | | | | |
| HCIM091613 | NM_001 | GLGAQ | 19 | GLGAQDGRGDRVR | 88 | 175486 |
| | 128160.1 | DGRGD | | AGARLRRQPLGHRA | | |
| | Exon1_ | RVRAG | | GTGRRRLQHE | | |
| | 3rd_1 | | | | | |
| HCIM0916I4 | NM_001 | ARLRR | 20 | GLGAQDGRGDRVR | 88 | 175486 |
| | 128160.1_ | QPLGH | | AGARLRRQPLGHRA | | |
| | Exon1_ | RAGTG | | GTGRRRLQHE | | |
| | 3rd_2 | | | | | |
| HCIM094285 | NM_001 | EKQVS | 21 | EKQVSMARLAPQGS | 89 | 70106 |
| | 130103.1_ | MARLA | | QET | | |
| | Exon23_ | PQGSQ | | | | |
| | 2nd_1 | | | | | |
| HCIM096912 | NM 001 | WRVRG | 22 | WRVRGCGRPLGAG | 90 | 221420 |
| | 134231.1_ | CGRPL | | CCAEATAGREPPRP | | |
| | Exon1_ | GAGCC | | RPPALGAAPRVPAP | | |
| | 2nd_1 | | | TAPASRAPRPPRHPP | | |
| | | | | AAPTSARTYGLATR | | |
| | | | | TCGDWCT | | |
| HCIM096913 | NM_001 | AEATA | 23 | WRVRGCGRPLGAG | 90 | 221420 |
| | 134231.1 | GREPPR | | CCAEATAGREPPRP | | |
| | Exon1_ | PRPP | | RPPALGAAPRVPAP | | |
| | 2nd_2 | | | TAPASRAPRPPRHPP | | |
| | | | | AAPTSARTYGLATR | | |
| | | | | TCGDWCT | | |
| HCIM096914 | NM_001 | ALGAA | 24 | WRVRGCGRPLGAG | 90 | 221420 |
| | 134231.1_ | PRVPAP | | CCAEATAGREPPRP | | |
| | Exon1_ | TAPA | | RPPALGAAPRVPAP | | |
| | 2nd_3 | | | TAPASRAPRPPRHPP | | |
| | | | | AAPTSARTYGLATR | | |
| | | | | TCGDWCT | | |
| HCIM096915 | NM_001 | SRAPRP | 25 | WRVRGCGRPLGAG | 90 | 221420 |
| | 134231.1_ | PRHPPA | | CCAEATAGREPPRP | | |
| | Exon1_ | APT | | RPPALGAAPRVPAP | | |
| | 2nd_4 | | | TAPASRAPRPPRHPP | | |
| | | | | AAPTSARTYGLATR | | |
| | | | | TCGDWCT | | |
| HCIM096916 | NM_001 | SARTY | 26 | WRVRGCGRPLGAG | 90 | 221420 |
| | 134231.1_ | GLATR | | CCAEATAGREPPRP | | |
| | Exon 1_ | TCGDW | | RPPALGAAPRVPAP | | |
| | 2nd_5 | | | TAPASRAPRPPRHPP | | |
| | | | | AAPTSARTYGLATR | | |
| | | | | TCGDWCT | | |
| HCIM107300 | NM_001 | GLGAE | 27 | GLGAELCLQVQVLR | 91 | 162032 |
| | 143838.2_ | LCLQV | | DLVRHPAPAAATRH | | |
| | Exon1_ | QVLRD | | SDARQ | | |
| | 3rd_1 | | | | | |
| HCIM107301 | NM_001 | LVRHP | 28 | GLGAELCLQVQVLR | 91 | 162032 |
| | 143838.2_ | APAAA | | DLVRHPAPAAATRH | | |
| | Exon1_ | TRHSD | | SDARQ | | |
| | 3rd_2 | | | | | |
| HCIM113647 | NM_001 | SGTRHT | 29 | PLPPQVPTAGATGK | 92 | 224396 |
| | 145770.2_ | GAAST | | TFASAASGTRHTGA | | |
| | Exon4_ | TNPH | | ASTTNPHQTCASPSR | | |
| | 2nd_2 | | | TPKRPSQSMPLSLQP | | |
| | | | | TLLPDPSLTPGASTT | | |
| | | | | SASTGTDMLGDYIFS | | |
| | | | | MASVTSC | | |
| HCIM113647 | NM_001 | SGTRHT | 29 | VPTAGATGKTFASA | 93 | 223783 |
| | 145770.2_ | GAAST | | ASGTRHTGAASTTN | | |
| | Exon4_ | TNPH | | PHQTCASPSRTPKRP | | |
| | 2nd_2 | | | SQSMPLSLQPTLLPD | | |
| | | | | PSLTPGASTTSASTG | | |
| | | | | TDMLGDYIFSMASV | | |
| | | | | TSC | | |
| HCIM113648 | NM_001 | QTCASP | 30 | PLPPQVPTAGATGK | 92 | 224396 |
| | 145770.2_ | SRTPKR | | TFASAASGTRHTGA | | |
| | Exon4_ | PSQ | | ASTTNPHQTCASPSR | | |
| | 2nd_3 | | | TPKRPSQSMPLSLQP | | |
| | | | | TLLPDPSLTPGASTT | | |
| | | | | SASTGTDMLGDYIFS | | |
| | | | | MASVTSC | | |
| HCIM113648 | NM_001 | QTCASP | 30 | VPTAGATGKTFASA | 93 | 223783 |
| | 145770.2_ | SRTPKR | | ASGTRHTGAASTTN | | |
| | Exon4_ | PSQ | | PHQTCASPSRTPKRP | | |
| | 2nd_3 | | | SQSMPLSLQPTLLPD | | |
| | | | | PSLTPGASTTSASTG | | |
| | | | | TDMLGDYIFSMASV | | |
| | | | | TSC | | |
| HCIM113649 | NM_001 | SMPLSL | 31 | PLPPQVPTAGATGK | 92 | 224396 |
| | 145770.2_ | QPTLLP | | TFASAASGTRHTGA | | |
| | Exon4_ | DPS | | ASTTNPHQTCASPSR | | |
| | 2nd_4 | | | TPKRPSQSMPLSLQP | | |
| | | | | TLLPDPSLTPGASTT | | |
| | | | | SASTGTDMLGDYIFS | | |
| | | | | MASVTSC | | |
| HCIM113649 | NM_001 | SMPLSL | 31 | VPTAGATGKTFASA | 93 | 223783 |
| | 145770.2_ | QPTLLP | | ASGTRHTGAASTTN | | |
| | _Exon4_ | DPS | | PHQTCASPSRTPKRP | | |
| | 2nd_4 | | | SQSMPLSLQPTLLPD | | |
| | | | | PSLTPGASTTSASTG | | |
| | | | | TDMLGDYIFSMASV | | |
| | | | | TSC | | |
| HCIM113650 | NM_001 | LTPGAS | 32 | VPTAGATGKTFASA | 93 | 223783 |
| | 145770.2_ | TTSAST | | ASGTRHTGAASTTN | | |
| | Exon4_ | GTD | | PHQTCASPSRTPKRP | | |
| | 2nd_5 | | | SQSMPLSLQPTLLPD | | |
| | | | | PSLTPGASTTSASTG | | |
| | | | | TDMLGDYIFSMASV | | |
| | | | | TSC | | |
| HCIM113650 | NM_001 | LTPGAS | 32 | PLPPQVPTAGATGK | 92 | 224396 |
| | 145770.2_ | TTSAST | | TFASAASGTRHTGA | | |
| | Exon4_ | GTD | | ASTTNPHQTCASPSR | | |
| | 2nd_5 | | | TPKRPSQSMPLSLQP | | |
| | | | | TLLPDPSLTPGASTT | | |
| | | | | SASTGTDMLGDYIFS | | |
| | | | | MASVTSC | | |
| HCIM113651 | NM_001 | MLGDY | 33 | VPTAGATGKTFASA | 93 | 223783 |
| | 145770.2_ | IFSMAS | | ASGTRHTGAASTTN | | |
| | Exon4 | VTSC | | PHQTCASPSRTPKRP | | |
| | 2nd_6 | | | SQSMPLSLQPTLLPD | | |
| | | | | PSLTPGASTTSASTG | | |
| | | | | TDMLGDYIFSMASV | | |
| | | | | TSC | | |
| HCIM113651 | NM_001 | MLGDY | 33 | PLPPQVPTAGATGK | 92 | 224396 |
| | 145770.2_ | IFSMAS | | TFASAASGTRHTGA | | |
| | Exon4_ | VTSC | | ASTTNPHQTCASPSR | | |
| | 2nd_6 | | | TPKRPSQSMPLSLQP | | |
| | | | | TLLPDPSLTPGASTT | | |
| | | | | SASTGTDMLGDYIFS | | |
| | | | | MASVTSC | | |
| HCIM113669 | NM_001 | PLPPQV | 34 | PLPPQVPTAGATGK | 92 | 224396 |
| | 145773.2_ | PTAGA | | TFASAASGTRHTGA | | |
| | Exon3_ | TGKT | | ASTTNPHQTCASPSR | | |
| | 2nd_1 | | | TPKRPSQSMPLSLQP | | |
| | | | | TLLPDPSLTPGASTT | | |
| | | | | SASTGTDMLGDYIFS | | |
| | | | | MASVTSC | | |
| HCIM113670 | NM_001 | FASAAS | 35 | PLPPQVPTAGATGK | 92 | 224396 |
| | 145773.2_ | GTRHT | | TFASAASGTRHTGA | | |
| | Exon3_ | GAAS | | ASTTNPHQTCASPSR | | |
| | 2nd_2 | | | TPKRPSQSMPLSLQP | | |
| | | | | TLLPDPSLTPGASTT | | |
| | | | | SASTGTDMLGDYIFS | | |
| | | | | MASVTSC | | |
| HCIM113670 | NM_001 | FASAAS | 35 | VPTAGATGKTFASA | 93 | 223783 |
| | 145773.2_ | GTRHT | | ASGTRHTGAASTTN | | |
| | Exon3_ | GAAS | | PHQTCASPSRTPKRP | | |
| | 2nd_2 | | | SQSMPLSLQPTLLPD | | |
| | | | | PSLTPGASTTSASTG | | |
| | | | | TDMLGDYIFSMASV | | |
| | | | | TSC | | |
| HCIM113671 | NM_001 | TTNPH | 36 | PLPPQVPTAGATGK | 92 | 224396 |
| | 145773.2_ | QTCASP | | TFASAASGTRHTGA | | |
| | Exon3_ | SRTP | | ASTTNPHQTCASPSR | | |
| | 2nd_3 | | | TPKRPSQSMPLSLQP | | |
| | | | | TLLPDPSLTPGASTT | | |
| | | | | SASTGTDMLGDYIFS | | |
| | | | | MASVTSC | | |
| HCIM113671 | NM_001 | TTNPH | 36 | VPTAGATGKTFASA | 93 | 223783 |
| | 145773.2_ | QTCASP | | ASGTRHTGAASTTN | | |
| | Exon3_ | SRTP | | PHQTCASPSRTPKRP | | |
| | 2nd_3 | | | SQSMPLSLQPTLLPD | | |
| | | | | PSLTPGASTTSASTG | | |
| | | | | TDMLGDYIFSMASV | | |
| | | | | TSC | | |
| HCIM113672 | NM_001 | KRPSQS | 37 | VPTAGATGKTFASA | 93 | 223783 |
| | 145773.2_ | MPLSL | | ASGTRHTGAASTTN | | |
| | Exon3_ | QPTL | | PHQTCASPSRTPKRP | | |
| | 2nd_4 | | | SQSMPLSLQPTLLPD | | |
| | | | | PSLTPGASTTSASTG | | |
| | | | | TDMLGDYIFSMASV | | |
| | | | | TSC | | |
| HCIM113672 | NM_001 | KRPSQS | 37 | PLPPQVPTAGATGK | 92 | 224396 |
| | 145773.2_ | MPLSL | | TFASAASGTRHTGA | | |
| | Exon3_ | QPTL | | ASTTNPHQTCASPSR | | |
| | 2nd_4 | | | TPKRPSQSMPLSLQP | | |
| | | | | TLLPDPSLTPGASTT | | |
| | | | | SASTGTDMLGDYIFS | | |
| | | | | MASVTSC | | |
| HCIM113673 | NM_001 | LPDPSL | 38 | VPTAGATGKTFASA | 93 | 223783 |
| | 145773.2_ | TPGAST | | ASGTRHTGAASTTN | | |
| | Exon3_ | TSA | | PHQTCASPSRTPKRP | | |
| | 2nd_5 | | | SQSMPLSLQPTLLPD | | |
| | | | | PSLTPGASTTSASTG | | |
| | | | | TDMLGDYIFSMASV | | |
| | | | | TSC | | |
| HCIM113673 | NM_001 | LPDPSL | 38 | PLPPQVPTAGATGK | 92 | 224396 |
| | 145773.2_ | TPGAST | | TFASAASGTRHTGA | | |
| | Exon3_ | TSA | | ASTTNPHQTCASPSR | | |
| | 2nd_5 | | | TPKRPSQSMPLSLQP | | |
| | | | | TLLPDPSLTPGASTT | | |
| | | | | SASTGTDMLGDYIFS | | |
| | | | | MASVTSC | | |
| HCIM113674 | NM_001 | STGTD | 39 | PLPPQVPTAGATGK | 92 | 224396 |
| | 145773.2_ | MLGDY | | TFASAASGTRHTGA | | |
| | Exon3_ | IFSMA | | ASTTNPHQTCASPSR | | |
| | 2nd_6 | | | TPKRPSQSMPLSLQP | | |
| | | | | TLLPDPSLTPGASTT | | |
| | | | | SASTGTDMLGDYIFS | | |
| | | | | MASVTSC | | |
| HCIM113674 | NM_001 | STGTD | 39 | VPTAGATGKTFASA | 93 | 223783 |
| | 145773.2_ | MLGDY | | ASGTRHTGAASTTN | | |
| | Exon3_ | IFSMA | | PHQTCASPSRTPKRP | | |
| | 2nd_6 | | | SQSMPLSLQPTLLPD | | |
| | | | | PSLTPGASTTSASTG | | |
| | | | | TDMLGDYIFSMASV | | |
| | | | | TSC | | |
| HCIM115998 | NM_001 | EIHLGT | 40 | EIHLGTIRKLFCCSV | 94 | 217745 |
| | 154.3_ | IRKLFC | | EKMTNVSRGRAPCC | | |
| | Exon13_ | CSV | | VPAPPHCLPSAPLAA | | |
| | 2nd_1 | | | FVCQCLTHCLIHTS | | |
| | | | | MLMTNTYTS | | |
| HCIM115999 | NM_001 | EKMTN | 41 | EIHLGTIRKLFCCSV | 94 | 217745 |
| | 154.3_Ex | VSRGR | | EKMTNVSRGRAPCC | | |
| | on_13_2n | APCCV | | VPAPPHCLPSAPLAA | | |
| | d_2 | | | FVCQCLTHCLIHTS | | |
| | | | | MLMTNTYTS | | |
| HCIM116000 | NM_001 | PAPPHC | 42 | EIHLGTIRKLFCCSV | 94 | 217745 |
| | 154.3_Ex | LPSAPL | | EKMTNVSRGRAPCC | | |
| | on_13_2n | AAF | | VPAPPHCLPSAPLAA | | |
| | d_3 | | | FVCQCLTHCLIHTS | | |
| | | | | MLMTNTYTS | | |
| HCIM116001 | NM_001 | VCQCL | 43 | EIHLGTIRKLFCCSV | 94 | 217745 |
| | l54.3_Ex | THCLIH | | EKMTNVSRGRAPCC | | |
| | on_13_ | TSML | | VPAPPHCLPSAPLAA | | |
| | 2nd_4 | | | FVCQCLTHCLIHTS | | |
| | | | | MLMTNTYTS | | |
| HCIM147367 | NM_001 | WPPPPP | 44 | WPPPPPAPVSPTTTS | 95 | 105081 |
| | 204492.1_ | APVSPT | | SSRSLA | | |
| | Exon1_ | TTS | | | | |
| | 2nd_1 | | | | | |
| HCIM188007 | NM_001 | GNPRA | 45 | GNPRALRAGGHHA | 96 | 224368 |
| | 287444.1_ | LRAGG | | RQDHRGVPQRGPVL | | |
| | Exon1_ | HHARQ | | RGQEVRAVAAPRG | | |
| | 3rd_l | | | HLRGAAGAAHGAG | | |
| | | | | GRPVRRAPPLHAHA | | |
| | | | | WAPGAGAGRAAGG | | |
| | | | | RQVRGGGPRALQG | | |
| | | | | AR | | |
| HCIM188008 | NM_001 | DHRGV | 46 | GNPRALRAGGHHA | 96 | 224368 |
| | 287444.1_ | PQRGP | | RQDHRGVPQRGPVL | | |
| | Exon1_ | VLRGQ | | RGQEVRAVAAPRG | | |
| | 3rd_2 | | | HLRGAAGAAHGAG | | |
| | | | | GRPVRRAPPLHAHA | | |
| | | | | WAPGAGAGRAAGG | | |
| | | | | RQVRGGGPRALQG | | |
| | | | | AR | | |
| HCIM188009 | NM_001 | EVRAV | 47 | GNPRALRAGGHHA | 96 | 224368 |
| | 287444.1_ | AAPRG | | RQDHRGVPQRGPVL | | |
| | Exon1_ | HLRGA | | RGQEVRAVAAPRG | | |
| | 3rd_3 | | | HLRGAAGAAHGAG | | |
| | | | | GRPVRRAPPLHAHA | | |
| | | | | WAPGAGAGRAAGG | | |
| | | | | RQVRGGGPRALQG | | |
| | | | | AR | | |
| HCIM188010 | NM_001 | AGAAH | 48 | GNPRALRAGGHHA | 96 | 224368 |
| | 287444.1 | GAGGR | | RQDHRGVPQRGPVL | | |
| | Exon1_ | PVRRA | | RGQEVRAVAAPRG | | |
| | 3rd_4 | | | HLRGAAGAAHGAG | | |
| | | | | GRPVRRAPPLHAHA | | |
| | | | | WAPGAGAGRAAGG | | |
| | | | | RQVRGGGPRALQG | | |
| | | | | AR | | |
| HCIM188011 | NM_001 | PPLHAH | 49 | GNPRALRAGGHHA | 96 | 224368 |
| | 287444.1_ | AWAPG | | RQDHRGVPQRGPVL | | |
| | Exon1_ | AGAG | | RGQEVRAVAAPRG | | |
| | 3rd_5 | | | HLRGAAGAAHGAG | | |
| | | | | GRPVRRAPPLHAHA | | |
| | | | | WAPGAGAGRAAGG | | |
| | | | | RQVRGGGPRALQG | | |
| | | | | AR | | |
| HCIM188012 | NM_001 | RAAGG | 50 | GNPRALRAGGHHA | 96 | 224368 |
| | 287444.1_ | RQVRG | | RQDHRGVPQRGPVL | | |
| | Exon1_ | GGPRA | | RGQEVRAVAAPRG | | |
| | 3rd_6 | | | HLRGAAGAAHGAG | | |
| | | | | GRPVRRAPPLHAHA | | |
| | | | | WAPGAGAGRAAGG | | |
| | | | | RQVRGGGPRALQG | | |
| | | | | AR | | |
| HCIM192261 | NM_001 | VPTAG | 51 | VPTAGATGKTFASA | 96 | 223783 |
| | 290142.1_ | ATGKT | | ASGTRHTGAASTTN | | |
| | Exon4_ | FASAA | | PHQTCASPSRTPKRP | | |
| | 2nd_1 | | | SQSMPLSLQPTLLPD | | |
| | | | | PSLTPGASTTSASTG | | |
| | | | | TDMLGDYIFSMASV | | |
| | | | | TSC | | |
| HCIM192261 | NM_001 | VPTAG | 51 | PLPPQVPTAGATGK | 96 | 224396 |
| | 290142.1_ | ATGKT | | TFASAASGTRHTGA | | |
| | Exon4_ | FASAA | | ASTTNPHQTCASPSR | | |
| | 2nd_1 | | | TPKRPSQSMPLSLQP | | |
| | | | | TLLPDPSLTPGASTT | | |
| | | | | SASTGTDMLGDYIFS | | |
| | | | | MASVTSC | | |
| HCIM209885 | NM_001_ | ERQQQ | 52 | ERQQQQSGPASLAG | 97 | 78439 |
| | 308333.1_ | QSGPAS | | FSVH | | |
| | Exon1_ | LAGF | | | | |
| | 3rd_1 | | | | | |
| HCIM229308 | NM_001 | TPSGTT | 53 | TPSGTTCHCIVDSCG | 98 | 227964 |
| | 455.3_ | CHCIVD | | SRMRELARALGGSS | | |
| | Exon2_ | SCG | | TLMGGRAEKPPGGG | | |
| | 2nd_1 | | | LSPWTIATSIPRAVA | | |
| | | | | AQPRRRQPCRQPPN | | |
| | | | | QLTTVPPSSPSGLAA | | |
| | | | | PRHAAVMSWMRGR | | |
| | | | | TSVHAPILTPAQSVA | | |
| | | | | AC'RPSWQAQSWMK | | |
| | | | | SRTMMRLSRPC'STA | | |
| | | | | AQPACHLQ | | |
| HCIM229309 | NM_001 | SRPCST | 54 | TPSGTTCHCIVDSCG | 98 | 227964 |
| | 455.3_ | AAQPA | | SRMRELARALGGSS | | |
| | Exon2_ | CHLQ | | TLMGGRAEKPPGGG | | |
| | 2nd_10 | | | LSPWTIATSIPRAVA | | |
| | | | | AQPRRRQPCRQPPN | | |
| | | | | QLTTVPPSSPSGLAA | | |
| | | | | PRHAAVMSWMRGR | | |
| | | | | TSVHAPILTPAQSVA | | |
| | | | | ACRPSWQAQSWMK | | |
| | | | | SRTMMRLSRPCSTA | | |
| | | | | AQPACHLO | | |
| HCIM229310 | NM_001 | SRMRE | 55 | TPSGTTCHCIVDSCG | 98 | 227964 |
| | 455.3_Ex | LARAL | | SRMRELARALGGSS | | |
| | on2_2nd | GGSST | | TLMGGRAEKPPGGG | | |
| | 2 | | | LSPWTIATSIPRAVA | | |
| | | | | AQPRRRQPCRQPPN | | |
| | | | | QLTTVPPSSPSGLAA | | |
| | | | | PRHAAVMSWMRGR | | |
| | | | | TSVHAPILTPAQSVA | | |
| | | | | ACRPSWQAQSWMK | | |
| | | | | SRTMMRLSRPCSTA | | |
| | | | | AQPACHLQ | | |
| HCIM229311 | NM_001 | LMGGR | 56 | TPSGTTCHCIVDSCG | 98 | 227964 |
| | 455.3_Ex | AEKPPG | | SRMRELARALGGSS | | |
| | on2_ | GGLS | | TLMGGRAEKPPGGG | | |
| | 2nd_3 | | | LSPWTIATSIPRAVA | | |
| | | | | AQPRRRQPCRQPPN | | |
| | | | | QLTTVPPSSPSGLAA | | |
| | | | | PRHAAVMSWMRGR | | |
| | | | | TSVHAPILTPAQSVA | | |
| | | | | ACRPSWQAQSWMK | | |
| | | | | SRTMMRLSRPCSTA | | |
| | | | | AQPACHLQ | | |
| HCIM229312 | NM_001 | PWTIAT | 57 | TPSGTTCHCIVDSCG | 98 | 227964 |
| | 455.3_Ex | SIPRAV | | SRMRELARALGGSS | | |
| | on2_2nd_4 | AAQ | | TLMGGRAEKPPGGG | | |
| | | | | LSPWTIATSIPRAVA | | |
| | | | | AQPRRRQPCRQPPN | | |
| | | | | QLTTVPPSSPSGLAA | | |
| | | | | PRHAAVMSWMRGR | | |
| | | | | TSVHAPILTPAQSVA | | |
| | | | | ACRPSWQAQSWMK | | |
| | | | | SRTMMRLSRPCSTA | | |
| | | | | AQPACHLQ | | |
| HCIM229313 | NM_001 | PRRRQP | 58 | TPSGTTCHCIVDSCG | 98 | 227964 |
| | 455.3_Ex | CRQPPN | | SRMRELARALGGSS | | |
| | on2_2nd_5 | QLT | | TLMGGRAEKPPGGG | | |
| | | | | LSPWTIATSIPRAVA | | |
| | | | | AQPRRRQPCRQPPN | | |
| | | | | QLTTVPPSSPSGLAA | | |
| | | | | PRHAAVMSWMRGR | | |
| | | | | TSVHAPILTPAQSVA | | |
| | | | | ACRPSWQAQSWMK | | |
| | | | | SRTMMRLSRPCSTA | | |
| | | | | AQPACHLQ | | |
| HCIM229314 | NM_001 | TVPPSS | 59 | TPSGTTCHCIVDSCG | 98 | 227964 |
| | 455.3_Ex | PSGLAA | | SRMRELARALGGSS | | |
| | on2_2nd_ | PRH | | TLMGGRAEKPPGGG | | |
| | 6 | | | LSPWTIATSIPRAVA | | |
| | | | | AQPRRRQPCRQPPN | | |
| | | | | QLTTVPPSSPSGLAA | | |
| | | | | PRHAAVMSWMRGR | | |
| | | | | TSVHAPILTPAQSVA | | |
| | | | | ACRPSWQAQSWMK | | |
| | | | | SRTMMRLSRPCSTA | | |
| | | | | AQPACHLQ | | |
| HCIM229315 | NM_001 | AAVMS | 60 | TPSGTTCHCIVDSCG | 98 | 227964 |
| | 455.3_Ex | WMRGR | | SRMRELARALGGSS | | |
| | on2_2nd_ | TSVHA | | TLMGGRAEKPPGGG | | |
| | 1 | | | LSPWTIATSIPRAVA | | |
| | | | | AQPRRRQPCRQPPN | | |
| | | | | QLTTVPPSSPSGLAA | | |
| | | | | PRHAAVMSWMRGR | | |
| | | | | TSVHAPILTPAQSVA | | |
| | | | | ACRPSWQAQSWMK | | |
| | | | | SRTMMRLSRPCSTA | | |
| | | | | AQPACHLQ | | |
| HCIM229316 | NM_001 | PILTPA | 61 | TPSGTTCHCIVDSCG | 98 | 227964 |
| | 455.3_Ex | QSVAA | | SRMRELARALGGSS | | |
| | on2_2nd_ | CRPS | | TLMGGRAEKPPGGG | | |
| | 8 | | | LSPWTIATSIPRAVA | | |
| | | | | AQPRRRQPCRQPPN | | |
| | | | | QLTTVPPSSPSGLAA | | |
| | | | | PRHAAVMSWMRGR | | |
| | | | | TSVHAPILTPAQSVA | | |
| | | | | ACRPSWQAQSWMK | | |
| | | | | SRTMMRLSRPCSTA | | |
| | | | | AQPACHLQ | | |
| HCIM229317 | NM_001 | WQAQS | 62 | TPSGTTCHCIVDSCG | 98 | 227964 |
| | 455.3_Ex | WMKSR | | SRMRELARALGGSS | | |
| | on2_2nd_ | TMMRL | | TLMGGRAEKPPGGG | | |
| | 9 | | | LSPWTIATSIPRAVA | | |
| | | | | AQPRRRQPCRQPPN | | |
| | | | | QLTTVPPSSPSGLAA | | |
| | | | | PRHAAVMSWMRGR | | |
| | | | | TSVHAPILTPAQSVA | | |
| | | | | ACRPSWQAQSWMK | | |
| | | | | SRTMMRLSRPCSTA | | |
| | | | | AQPACHLQ | | |
| HCIM238804 | NM_002 | EREVIQ | 63 | EREVIQERNRNFRQ | 99 | 213470 |
| | 423.4_Ex | ERNRN | | NIHSFIHWIVYHCCT | | |
| | on6_2nd_ | FRQN | | IRIDKHCSSTPFSNY | | |
| | 1 | | | VTLFYCSWFLNVFH | | |
| | | | | SF | | |
| HCIM238805 | NM_002 | IHSFIH | 64 | EREVIQERNRNFRQ | 99 | 213470 |
| | 423.4_Ex | WIVYH | | NIHSFIHWIVYHCCT | | |
| | on6_2nd_ | CCTI | | IRIDKHCSSTPFSNY | | |
| | 2 | | | VTLFYCSWFLNVFH | | |
| | | | | SF | | |
| HCIM238806 | NM_002 | RIDKHC | 65 | EREVIQERNRNFRQ | 99 | 213470 |
| | 423.4_Ex | SSTPFS | | NIHSFIHWIVYHCCT | | |
| | on6_2nd_ | NYV | | IRIDKHCSSTPFSNY | | |
| | 3 | | | VTLFYCSWFLNVFH | | |
| | | | | SF | | |
| HCIM238807 | NM_002 | TLFYCS | 66 | EREVIQERNRNFRQ | 99 | 213470 |
| | 423.4_Ex | WFLNV | | NIHSFIHWIVYHCCT | | |
| | on6_2nd | FHSF | | IRIDKHCSSTPFSNY | | |
| | _4 | | | VTLFYCSWFLNVFH | | |
| | | | | SF | | |
| HCIM248502 | NM_003 | LVPGA | 67 | LVPGATQSQWTLEG | 100 | 64577 |
| | 399.5_Ex | TQSQW | | RM | | |
| | on2_2nd | TLEGR | | | | |
| | 1 | | | | | |
| HCIM316305 | NM_017 | QYSKA | 68 | QYSKAAPGEGEGGS | 101 | 163373 |
| | 895.7_Ex | APGEG | | GPRAREELVPDQRR | | |
| | on_16_ | EGGSG | | EEEGE | | |
| | 3rd_1 | | | | | |
| HCIM316306 | NM_017 | PRAREE | 69 | QYSKAAPGEGEGGS | 101 | 163373 |
| | 895.7_Ex | LVPDQ | | GPRAREELVPDQRR | | |
| | on_16_3rd | RREE | | EEEGE | | |
| | 2 | | | | | |
| HCIM388991 | NM_199 | GCCEE | 70 | GCCEEVFCRVSCIIH | 102 | 165429 |
| | 344.2_Ex | VFCRVS | | GQFYEALEGTMDRS | | |
| | on8_3rd_ | CIIH | | WWTVL | | |
| | 1 | | | | | |
| HCIM388992 | NM_199 | GQFYE | 71 | GCCEEVFCRVSCIIH | 102 | 165429 |
| | 344.2_Ex | ALEGT | | GQFYEALEGTMDRS | | |
| | on8_3rd_ | MDRSW | | WWTVL | | |
| | 2 | | | | | |
| HCIM391774 | NM_212 | GVPGSS | 72 | GVPGSSAEAPAEAG | 103 | 226620 |
| | 550.4_Ex | AEAPA | | DGGAGGGDRDGFR | | |
| | on2_3rd_ | EAGD | | ALCVLVGGGGAVP | | |
| | 1 | | | GSFGPDARPPHGAA | | |
| | | | | GGWGSRGDRLGAG | | |
| | | | | AGAGTDGRAEGPAS | | |
| | | | | TRGAAGIGGGGLGH | | |
| | | | | GGGPGARPRALAPA | | |
| | | | | TSAGGEPGAAGPRR | | |
| | | | | GGRRERCLPPCRPR | | |
| | | | | RGRPG | | |
| HCIM391775 | NM_212 | GGAGG | 73 | GVPGSSAEAPAEAG | 103 | 226620 |
| | 550.4_Ex | GDRDG | | DGGAGGGDRDGFR | | |
| | on2_3rd_ | FRALC | | ALCVLVGGGGAVP | | |
| | 2 | | | GSFGPDARPPHGAA | | |
| | | | | GGWGSRGDRLGAG | | |
| | | | | AGAGTDGRAEGPAS | | |
| | | | | TRGAAGIGGGGLGH | | |
| | | | | GGGPGARPRALAPA | | |
| | | | | TSAGGEPGAAGPRR | | |
| | | | | GGRRERCLPPCRPR | | |
| | | | | RGRPG | | |
| HCIM39I776 | NM_212 | VLVGG | 74 | GVPGSSAEAPAEAG | 103 | 226620 |
| | 550.4_Ex | GGAVP | | DGGAGGGDRDGFR | | |
| | on2_3rd_ | GSFGP | | ALCVLVGGGGAVP | | |
| | 3 | | | GSFGPDARPPHGAA | | |
| | | | | GGWGSRGDRLGAG | | |
| | | | | AGAGTDGRAEGPAS | | |
| | | | | TRGAAGIGGGGLGH | | |
| | | | | GGGPGARPRALAPA | | |
| | | | | TSAGGEPGAAGPRR | | |
| | | | | GGRRERCLPPCRPR | | |
| | | | | RGRPG | | |
| HCIM391777 | NM_212 | DARPP | 75 | GVPGSSAEAPAEAG | 103 | 226620 |
| | 550.4_Ex | HGAAG | | DGGAGGGDRDGFR | | |
| | on2_3rd_ | GWGSR | | ALCVLVGGGGAVP | | |
| | 4 | | | GSFGPDARPPHGAA | | |
| | | | | GGWGSRGDRLGAG | | |
| | | | | AGAGTDGRAEGPAS | | |
| | | | | TRGAAGIGGGGLGH | | |
| | | | | GGGPGARPRALAPA | | |
| | | | | TSAGGEPGAAGPRR | | |
| | | | | GGRRERCLPPCRPR | | |
| | | | | RGRPG | | |
| HCIM391778 | NM212 | GDRLG | 76 | GVPGSSAEAPAEAG | 103 | 226620 |
| | 550.4_Ex | AGAGA | | DGGAGGGDRDGFR | | |
| | on2_3rd_ | GTDGR | | ALCVLVGGGGAVP | | |
| | 5 | | | GSFGPDARPPHGAA | | |
| | | | | GGWGSRGDRLGAG | | |
| | | | | AGAGTDGRAEGPAS | | |
| | | | | TRGAAGIGGGGLGH | | |
| | | | | GGGPGARPRALAPA | | |
| | | | | TSAGGEPGAAGPRR | | |
| | | | | GGRRERCLPPCRPR | | |
| | | | | RGRPG | | |
| HCIM391779 | NM_212 | AEGPAS | 77 | GVPGSSAEAPAEAG | 103 | 226620 |
| | 550.4_Ex | TRGAA | | DGGAGGGDRDGFR | | |
| | on2_3rd_ | GIGG | | ALCVLVGGGGAVP | | |
| | 6 | | | GSFGPDARPPHGAA | | |
| | | | | GGWGSRGDRLGAG | | |
| | | | | AGAGTDGRAEGPAS | | |
| | | | | TRGAAGIGGGGLGH | | |
| | | | | GGGPGARPRALAPA | | |
| | | | | TSAGGEPGAAGPRR | | |
| | | | | GGRRERCLPPCRPR | | |
| | | | | RGRPG | | |
| HCIM391780 | NM_212 | GGLGH | 78 | GVPGSSAEAPAEAG | 103 | 226620 |
| | 550.4_Ex | GGGPG | | DGGAGGGDRDGFR | | |
| | on2_3rd_ | ARPRA | | ALCVLVGGGGAVP | | |
| | 7 | | | GSFGPDARPPHGAA | | |
| | | | | GGWGSRGDRLGAG | | |
| | | | | AGAGTDGRAEGPAS | | |
| | | | | TRGAAGIGGGGLGH | | |
| | | | | GGGPGARPRALAPA | | |
| | | | | TSAGGEPGAAGPRR | | |
| | | | | GGRRERCLPPCRPR | | |
| | | | | RGRPG | | |
| HCIM391781 | NM_212 | LAPATS | 79 | GVPGSSAEAPAEAG | 103 | 226620 |
| | 550.4_ | AGGEP | | DGGAGGGDRDGFR | | |
| | Exon2_ | GAAG | | ALCVLVGGGGAVP | | |
| | 3rd_8 | | | GSFGPDARPPHGAA | | |
| | | | | GGWGSRGDRLGAG | | |
| | | | | AGAGTDGRAEGPAS | | |
| | | | | TRGAAGIGGGGLGH | | |
| | | | | GGGPGARPRALAPA | | |
| | | | | TSAGGEPGAAGPRR | | |
| | | | | GGRRERCLPPCRPR | | |
| | | | | RGRPG | | |
| HCIM391782 | NM_212 | PRRGG | 80 | GVPGSSAEAPAEAG | 103 | 226620 |
| | 550.4_ | RRERCL | | DGGAGGGDRDGFR | | |
| | Exon2 | PPCR | | ALCVLVGGGGAVP | | |
| | 3rd_9 | | | GSFGPDARPPHGAA | | |
| | | | | GGWGSRGDRLGAG | | |
| | | | | AGAGTDGRAEGPAS | | |
| | | | | TRGAAGIGGGGLGH | | |
| | | | | GGGPGARPRALAPA | | |
| | | | | TSAGGEPGAAGPRR | | |
| | | | | GGRRERCLPPCRPR | | |
| | | | | RGRPG | | |

TABLE 2

| | SEQ |
|---|---|
| | ID |
| frameshift | NO: |
| PQVGKKLWQLCWIMTCLRLFTRNHFAVILL | 81 |
| MYQKSPGRLVDGSSW | |
| HEAGLGLHLEGTLWPGPHHRTLELPTEPDP | 82 |
| GAPGGRPRR | |
| PSTGCPPPGKALLQGFLHRHSEAAGAYHRL | 83 |
| QLRPLPGHQWQARKEDRWRLERHDRG | |
| GRATLQRGGFGAGAGRAVRSGRGAADRHR | 84 |
| VSRHVALGLPHLGSLQWAPTSGSSPTQPWE | 85 |
| EEPLDWSQSLSQTHQTKERGFEGTLKIHR | 86 |
| GQPSLAAGVLRPRLAGGLSAAQITGREPR | |
| HPRTGLQLCRRARRPQRVCGE | |
| VVCQEGWSGPLLAQRLVPASLGQSQPGIT | 87 |
| ASLCPPQCRE | |
| GLGAQDGRGDRVRAGARLRRQPLGHRAGT | 88 |
| GRRRLQHE | |
| EKQVSMARLAPQGSQET | 89 |
| WRVRGCGRPLGAGCCAEATAGREPPRPRP | 90 |
| PALGAAPRVPAPTAPASRAPRPPRHPPAA | |
| PTSARTYGLATRTCGDWCT | |
| GLGAELCLQVQVLRDLVRHPAPAAATRHS | 91 |
| DARQ | |
| PLPPQVPTAGATGKTFASAASGTRHTGAA | 92 |
| STTNPHQTCASPSRTPKRPSQSMPLSLQP | |
| TLLPDPSLTPGASTTSASTGTDMLGDYIF | |
| SMASVTSC | |
| VPTAGATGKTFASAASGTRHTGAASTTNP | 93 |
| HQTCASPSRTPKRPSQSMPLSLQPTLLPD | |
| PSLTPGASTTSASTGTDMLGDYIFSMASV | |
| TSC | |
| EIHLGTIRKLFCCSVEKMTNVSRGRAPCC | 94 |
| VPAPPHCLPSAPLAAFVCQCLTHCLIHTS | |
| MLMTNTYTS | |
| WPPPPPAPVSPTTTSSSRSLA | 95 |
| GNPRALRAGGHHARQDHRGVPQRGPVLRG | 96 |
| QEVRAVAAPRGHLRGAAGAAHGAGGRPVR | |
| RAPPLHAHAWAPGAGAGRAAGGRQVRGGG | |
| PRALQGAR | |
| ERQQQQSGPASLAGFSVH | 97 |
| TPSGTTCHCIVDSCGSRMRELARALGGSS | 98 |
| TLMGGRAEKPPGGGLSPWTIATSIPRAVA | |
| AQPRRRQPCRQPPNQLTTVPPSSPSGLAA | |
| PRHAAVMSWMRGRTSVHAPILTPAQSVAA | |
| CRPSWQAQSWMKSRTMMRLSRPCSTAAQP | |
| ACHLQ | |
| EREVIQERNRNFRQNIHSFIHWIVYHCCT | 99 |
| IRIDKHCSSTPFSNYVTLFYCSWFLNVFH | |
| SF | |
| LVPGATQSQWTLEGRM | 100 |
| QYSKAAPGEGEGGSGPRAREELVPDQRRE | 101 |
| EEGE | |
| GCCEEVFCRVSCIIHGQFYEALEGTMDRS | 102 |
| WWTVL | |
| GVPGSSAEAPAEAGDGGAGGGDRDGFRAL | 103 |
| CVLVGGGGAVPGSFGPDARPPHGAAGGWG | |
| SRGDRLGAGAGAGTDGRAEGPASTRGAAG | |
| IGGGGLGHGGGPGARPRALAPATSAGGEP | |
| GAAGPRRGGRRERCLPPCRPRRGRPG | |
| AASSCMGPWCSSSLSSSTWWPSPSPH | 104 |
| CRRERCSWPRWPRWRWPGSPLRRRVPRAA | 105 |
| TCRGVPAPAAPAATCPTSATAAWCAPPAR | |
| ASPVAALWTRLAARAWSACAAYAAAAGRT | |
| PCVAPTGTPMPTCARCRRPAAARCSSPGR | |
| PCASCRRAPARW | |
| KQHVIYSNKKYISFWAQSINPVTEKRIQV | 106 |
| EQTRDEDLDTDSLD | |
| CHRQGDRRLGETCRLIFMSKH | 107 |
| STASASRRWMGTSEHPNAPRWAAASGAGI | 108 |
| WEDTAGSAGLLQGWPKASAAARSGTCPAS | |
| SLPPSGTSLATLAVMTSWLIGPGPAHSSH | |
| ACQKLEGCWRPIELMGAAGAWPSAPASPE | |
| PKGTSWRPHGATRLPRDGSACTFETPVSF | |
| NVHIPGDHTCLLLLILASRFTLTLY | |
| EKEEPLDQWESKVLRDAMAPKVL | 109 |
| LWALTLTSVCKFKRMEKSVWSAPQWDGTQ | 110 |
| SPRCSGELPRERSFHLHQSPGILMKKVCS | |
| LWLLQ | |
| EKYVEVSRRPVRA | 111 |
| EWQYFREASDSMPRFTPR1SSLLPPWA | 112 |
| YIQTRIEEMDHLNISFQEMEQEISSLLMK | 113 |
| TQATYRPPRGWTGKKNPFTSFELKL | |
| RSAEGAGARLGDDEGTGVSDERDAVLGRR | 114 |
| GAGAPAGRPLLPLLSRRLPGPGAPWRRGP | |
| PGPLGHAAQLSVPGGGRLRAGHSRAAARG | |
| RRDGDSSAGPGQETRVRASLVHERGVRRP | |
| GAAPEGREGVRQHQSPRYGGLREREDLLW | |
| GRDGGVREYECVVRVREYWGPGRPGPHGS | |
| GKNVGDCLEIDFEPDENKEWKASVLPI | |
| GAVDHSLPEEADKAEGSPWLSKASLQAKE | 115 |
| ASTSSGEKW | |
| ICSGPPASMPASSYPTSPSTASLGCCSSL | 116 |
| LLS | |
| ASGPGPRPSERAQATGHAAILPREALLGA | 117 |
| RLGLGPGNQNSVPYPAGALLEPSWGVVAG | |
| NPTGTSALPGLVPSWTACTCQPPTCFLKA | |
| TLAHTRMRPVQPAKGQSGGAASCQCPPQL | |
| CLPFSPARFNQNAMLCKSLLLGGG | |
| LISQMNYLWAPRMCTDSSPPGKTLRQWRQ | 118 |
| I | |
| LKDMTIVLMTTWKLEMEPVKIAL | 119 |
| LKPWSHSCDTSMGRSARAKSKRAWRLQHN | 120 |
| ASGPTRCWSHPVMRWR | |
| RLMMTPVPMTSSSGRFTSAAWMTCSSFWP | 121 |
| ARLLRSNGAYMC | |
| LPLHQRTSSSTASWSPGW | 122 |
| LPSLGARATTTQPVKTYVAEGQ | 123 |
| LQVQTKKKKGRRGRKEKQRTNL | 124 |
| LRAVRPGPEKPAGSLCWEQW | 125 |
| GGGWLLGAPGALWTSPPAQHHPVLPAAP | 126 |
| LWPLGSWLSLEPVLRAVRPGPEKPAGSL | |
| CWEQW | |
| LSNGGRLLQIWF | 127 |
| LVPDSPSSLAGTGTVCQGKWTQPWLAAS | 128 |
| TSQAWHPAPPWPRNKGLGIATAKATVHN | |
| EATAVAATRTPAGHPAPRGCPCSPVRRA | |
| TWEPTTMMTTGWTGLCLPPVNPSRVSSS | |
| SLE | |
| LVPSPSRNVPAELSMRRTSSRFTPSSFL | 129 |
| KE | |
| MKMFDHTSETDFACMICCPQIKYKDLSN | 130 |
| H | |
| NLFPVHAMCLNAAMTVF | 131 |
| NNWKESMMLFPVNMSAADEC | 132 |
| AICFLEEKAKQGLLKSLSPIWAVSPTWH | 133 |
| IGRASSWSISTTTMRGTSASRQLAERR | |
| ACAHMTLPPLCSTST | |
| SGRERMRKARRGRTPCPGPRPTGCVPS | 134 |
| TRCGCSCR | |
| LSLRKLQRRLGTLRPSHRMRRCCSSMAT | 135 |
| TNKQLWAT | |
| GPSGSGCPGSSVAMPRPTAAMAACPGRS | 136 |
| CSSPPSRCSEGGMWWPLSSAVSCTT | |
| ASCGLPCRRQPC | |
| SRMTSSTPVCSTSWTRSSLPCWQIPPVA | 137 |
| SFTWRLPSSPVGGTSRQMPHRKSCETLC | |
| AR | |
| SWDTENWSRDKWQ | 138 |
| TCRLPKCMGCFQGCIYFKTSLQHY | 139 |
| QTVLGRIHQRCQERPLHRPPAAVRPQQC | 140 |
| QSVLSERPLDSCRETQACRAV | |
| VTFWTEMPIMKEMEIIPICTRDTRRQKE | 141 |
| ELQ | |
| WAQASVLLMIPTDMQSMRTM | 142 |
| WSAQRSSETWSKPLTPCSL | 143 |
| WTRPWPSTLGMGQSWPSKKISTQPCQSPS | 144 |
| GSAHSATRTWSLRPRRMA | |
| WTSGGGLPTLAPPSTSECPSPASRRSRSF | 145 |
| WSPTASAMRP | |
| GVLLGPTSHGIGGDSRPRPRFRLRLQDQE | 146 |
| EAFRAAEGEGVPGGRPAGGCVPVGRSPL | |
| GSHGLPLTPLPPACSAGIGSCWRQPRPPR | 147 |
| TAGFPSREDPEGEAARPAAGSSLCLGLLG | |
| SLEPLHRWHHLGAETRRGGAHGRGLLPLH | |
| RGLPPAEASQLLRPLRVGGPAGEVASPGQ | |
| RAPLLAPGAGHTDTRHQLPQRDAAEQ | |
| ALLQEGVPLPRFPGQIPGFPEAGQTSSCP | 148 |
| HHQNHPPTGGTI | |
| ARGAGGSRARRPAVRHGQHPEEADWQSRR | 149 |
| RQAAGKGRAAAGPDPGGRRGQAEPTAGLC | |
| GGGGGRRRHRASGRRAGRGEPCRPEEPGQ | |
| RAVPKRAVRRGGRQVLGGNRAPGAS | |
| GVQREVSQQPQARHQGAPGHHRGQGTPGS | 150 |
| RWESPLDQALEEGGPGREAAAAS | |
| SPHHGTCQEDLGQGRPCPSQQPQQPEASP | |
| HGPQOSRCNPGAQGSWGRGGGR | |
| EDWPICRALQPAVEHQRRPFLVQSEPGSH | 151 |
| PHV | |
| FLLSTSLTSSTLREPGSRDSTPSMKSSPG | 152 |
| SWSPPSPSQPTSSDHLKKK | |
| KRPIKQWARRLQKGSLMKKEQTVKQWKKR | 153 |
| NLRATYDATPRREESQ | |
| TLGRRRRKGAAAVKDLTPLPLIGSSLGPG | 154 |
| ISCAAPSIALSTGSGGSRLTTWDRPLTVA | |
| HGSYPIPTEY | |
| LVTGATLATTLLPSRPHRMLVTGSSEPRQ | 155 |
| LQWLD | |

In some embodiments, the array is a glass slide or nitrocellulose membrane having in vitro synthesized peptides spotted in a predetermined pattern and screened for binding of antibodies in a biological sample, such as obtained from one or more subjects.

In some embodiments, detection of antibody binding on a peptide array poses some challenges that can be addressed by the technologies disclosed herein. Accordingly, in some embodiments, the arrays and methods disclosed herein utilize specific coatings and functional group densities on the surface of the array that can tune the desired properties necessary for performing assays. For example, non-specific antibody binding on a peptide array may be minimized by coating the silicon surface with a moderately hydrophilic monolayer polyethylene glycol (PEG), polyvinyl alcohol, carboxymethyl dextran, and combinations thereof. In some embodiments, the hydrophilic monolayer is homogeneous. Second, synthesized peptides are linked to the silicon surface using a spacer that moves the peptide away from the surface so that the peptide is presented to the antibody in an unhindered orientation.

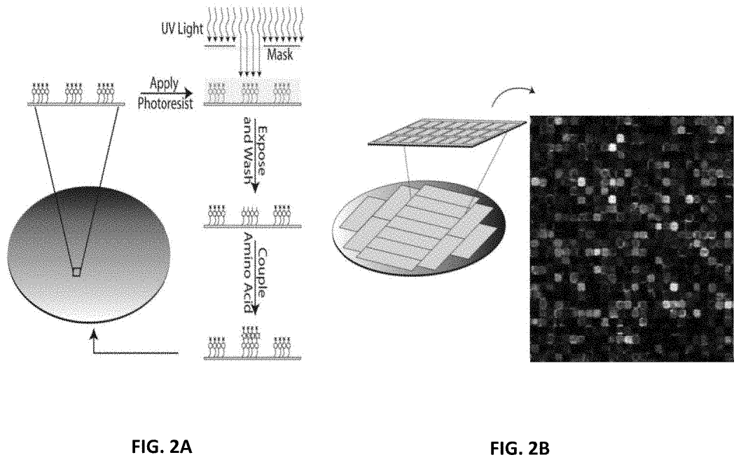

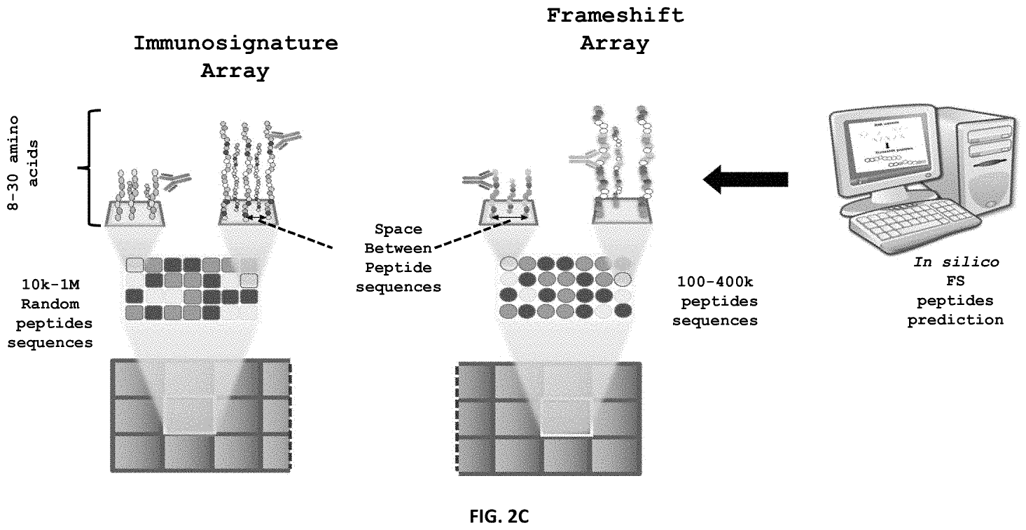

The spacing of the peptides in the features is a key feature (FIGS. 2A-2C). In order to detect the cognate binding of the FS peptide to the antibody it elicited in patient, the peptides need to be 3 nm or further apart. If the peptides are closer than 3 nm, then avidity becomes the dominant binding feature. Avidity is the basis of the immunosignaturing (IMS) effect. The inventors find that IMS can also classify MS-H, but not as well as the FSP arrays.

In some embodiments, the assay for MSI-H status is done with a standard ELISA. Since the antibodies detected on the FSP arrays are of high affinity, the peptides can also be used in an ELISA to detect the relevant antibody. This is not true for peptides in the IMS diagnostic as they are bound to the array by low affinity, avidity reactions which do not support use in ELISAs. As shown in Table 1, twenty-three or even fewer FSP chosen from the array can be synthesized and used in a standard ELISA. This assay type is commonly and widely used which would make the assay even more acceptable and another advantage of the disclosure.

TABLE 3
Frameshift peptides chosen from the 400K FSPs based on their
ability to classify MSI-H from MSS patients. These peptides
would also constitute a vaccine for MSI-H patients.

| | | peptide_ | SEQ ID | | SEQ ID | | |
|---|---|---|---|---|---|---|---|
| nimb_id | desc_id | sequence | NO: | frameshift | NO: | fsid | gene |
| HCIM | CCDS50 | MYQKS | 3 | PQVGKKLWQLCWIM | 81 | 235 | REV3L |
| 1151 | 91.2_del_3 | PGRLV | | TCLRLFTRNHFAVILL | | 570 | |
| 0 | | DGSSW | | MYQKSPGRLVDGSSW | | | |
| HCIM | NM_000 | GPHHR | 5 | HEAGLGLHLEGTLWP | 82 | 181 | ALDH3B2 |
| 3095 | 695.3_Ex | TLELPT | | GPHHRTLELPTEPDPG | | 308 | |
| 3 | on4_3rd_2 | EPDP | | APGGRPRR | | | |
| HCIM | NM_000 | QLRPLP | 8 | PSTGCPPPGKALLQGF | 83 | 210 | GRIN2D |
| 3369 | 836.2_Ex | GHQWQ | | LHRHSEAAGAYHRLQ | | 409 | |
| 2 | on6_3rd_3 | ARKE | | LRPLPGHQWQARKED | | | |
| | | | | RWRLERHDRG | | | |
| HCIM | NM_001 | GRATL | 9 | GRATLQRGGFGAGAG | 84 | 145 | SREBF1 |
| 4174 | 005291.2_ | QRGGF | | RAVRSGRGAADRHR | | 701 | |
| 0 | Exon1_3rd_1 | GAGAG | | | | | |
| HCIM | NM_001 | QWAPT | 11 | VSRHVALGLPHLGSL | 85 | 152 | SCYL1 |
| 6787 | 048218.1_ | SGSSPT | | QWAPTSGSSPTQPWE | | 624 | |
| 3 | Exon5_3rd_2 | QPWE | | | | | |
| HCIM | NM_001 | EEPLD | 12 | EEPLDWSQSLSQTHQT | 86 | 221 | CCDC88C |
| 7157 | 80414.3 | WSQSL | | KERGFEGTLKIHRGQP | | 751 | |
| 4 | Exon25_3rd_1 | SQTHQ | | SLAAGVLRPRLAGGL | | | |
| | | | | SAAQITGREPRHPRTG | | | |
| | | | | LQLCRRARRPQRVCG | | | |
| | | | | E | | | |
| HCIM | NM_001 | LVPASL | 18 | VVCQEGWSGPLLAQR | 87 | 182 | PHYHD1 |
| 8179 | 100876.1_ | GQSQP | | LVPASLGQSQPGITAS | | 759 | |
| 5 | Exon11_ | GITA | | LCPPQCRE | | | |
| | 2nd_2 | | | | | | |
| HCIM | NM_001 | GLGAQ | 19 | GLGAQDGRGDRVRA | 88 | 175 | UBP1 |
| 9161 | 128160.1_ | DGRGD | | GARLRRQPLGHRAGT | | 486 | |
| 3 | Exon1_ | RVRAG | | GRRRLQHE | | | |
| | 3rd_1 | | | | | | |
| HCIM | NM_001 | EKQVS | 21 | EKQVSMARLAPQGSQ | 89 | 701 | COL13A1 |
| 9428 | 130103.1_ | MARLA | | ET | | 6 | |
| 5 | Exon23_ | PQGSQ | | | | | |
| | 2nd_1 | | | | | | |
| HCIM | NM_001 | ALGAA | 24 | WRVRGCGRPLGAGCC | 90 | 221 | NT5DC2 |
| 9691 | 134231.1_ | PRVPAP | | AEATAGREPPRPRPPA | | 420 | |
| 4 | Exon1_ | TAPA | | LGAAPRVPAPTAPASR | | | |
| | 2nd_3 | | | APRPPRHPPAAPTSAR | | | |
| | | | | TYGLATRTCGDWCT | | | |
| HCIM | NM_001 | GLGAE | 27 | GLGAELCLQVQVLRD | 91 | 162 | SLC13A5 |
| 10730 | 143838.2_ | LCLQV | | LVRHPAPAAATRHSD | | 32 | |
| 0 | Exon1_ | QVLRD | | ARQ | | | |
| | 3rd_1 | | | | | | |
| HCIM | NM_001 | LTPGAS | 32 | VPTAGATGKTFASA A | 93 | 223 | ADGRG1 |
| 11365 | 145770.2_ | TTSAST | | SGTRHTGAASTTN PH | | 783 | |
| 0 | Exon4_ | GTD | | QTCASPSRTPKRPSQS | | | |
| | 2nd_5 | | | MPLSLQPTLLPDPSLT | | | |
| | | | | PGASTTSASTGTDML | | | |
| | | | | GDY1FSMASVTSC | | | |
| HCIM | NM_001 | LTPGAS | 32 | PLPPQVPTAGATGKTF | 92 | 224 | ADGRG1 |
| 11365 | 145770.2_ | TTSAST | | ASAASGTRHTGAAST | | 396 | |
| 0 | _Exon4_ | GTD | | TNPHQTCASPSRTPKR | | | |
| | 2nd_5 | | | PSQSMPLSLQPTLLPD | | | |
| | | | | PSLTPGASTTSASTGT | | | |
| | | | | DMLGDYIFSMASVTS | | | |
| | | | | C | | | |
| HCIM | NM_001 | EKMTN | 41 | EIHLGTIRKLFCCSVEK | 94 | 217 | ANXA5 |
| 11599 | 154.3_Ex | VSRGR | | MTNVSRGRAPCCVPA | | 745 | |
| 9 | on_13_2n | APCCV | | PPHCLPSAPLAAFVCQ | | | |
| | d_2 | | | CLTHCLIHTSMLMTNT | | | |
| | | | | YTS | | | |
| HCIM | NM_001 | WPPPPP | 44 | WPPPPPAPVSPTTTSSS | 95 | 105 | CAMK2G |
| 14736 | 204492.1_ | APVSPT | | RSLA | | 81 | |
| 7 | _Exon1_ | TTS | | | | | |
| | 2nd_1 | | | | | | |
| HCIM | NM_001 | PPLHAH | 49 | GNPRALRAGGHHARQ | 96 | 224 | DCDC2C |
| 18801 | 287444.1_ | AWAPG | | DHRGVPQRGPVLRGQ | | 368 | |
| 1 | Exon1_ | AGAG | | EVRAVAAPRGHLRGA | | | |
| | 3rd_5 | | | AGAAHGAGGRPVRR | | | |
| | | | | APPLHAHAWAPGAGA | | | |
| | | | | GRAAGGRQVRGGGPR | | | |
| | | | | ALQGAR | | | |
| HCIM | NM_001 | ERQQQ | 52 | ERQQQQSGPASLAGFS | 97 | 784 | POPDC2 |
| 20988 | 308333.1_ | QSGPAS | | VH | | 39 | |
| 5 | Exon1_ | LAGF | | | | | |
| | 3rd_1 | | | | | | |
| HCIM | NM_001 | LMGGR | 56 | TPSGTTCHCIVDSCGS | 98 | 227 | FOXO3 |
| 22931 | 455.3_Ex | AEKPPG | | RMRELARALGGSSTL | | 964 | |
| 1 | on2_2nd_3 | GGLS | | MGGRAEKPPGGGLSP | | | |
| | | | | WTIATSIPRAVAAQPR | | | |
| | | | | RRQPCRQPPNQLTTVP | | | |
| | | | | PSSPSGLAAPRHAAV | | | |
| | | | | MSWMRGRTSVHAPIL | | | |
| | | | | TPAQSVAACRPSWQA | | | |
| | | | | QSWMKSRTMMRLSR | | | |
| | | | | PCSTAAQPACHLQ | | | |
| HCIM | NM_002 | EREVIQ | 63 | EREVIQERNRNFRQNI | 99 | 213 | MMP7 |
| 23880 | 423.4_Ex | ERNRN | | HSFIHWIVYHCCTIRID | | 470 | |
| 4 | on6_2nd_1 | FRQN | | KHCSSTPFSNYVTLFY | | | |
| | | | | CSWFLNVFHSF | | | |
| HCIM | NM_003 | LVPGA | 67 | LVPGATQSQWTLEGR | 100 | 645 | XPNPEP2 |
| 24850 | 399.5_Ex | TQSQW | | M | | 77 | |
| 2 | on2_2nd_ | TLEGR | | | | | |
| | 1 | | | | | | |
| HCIM | NM_017 | QYSKA | 68 | QYSKAAPGEGEGGSG | 101 | 163 | DDX27 |
| 31630 | 895.7_Ex | APGEG | | PRAREELVPDQRREEE | | 373 | |
| 5 | on_16_3rd_ | EGGSG | | GE | | | |
| | 1 | | | | | | |
| HCIM | NM_199 | GQFYE | 71 | GCCEEVFCRVSCIIHG | 102 | 165 | SFT2D2 |
| 38899 | 344.2_Ex | ALEGT | | QFYEALEGTMDRSW | | 429 | |
| 2 | on8_3rd_ | MDRSW | | WTVL | | | |
| | 2 | | | | | | |
| HCIM | NM_212 | GDRLG | 76 | GVPGSSAEAPAEAGD | 103 | 226 | BLOC1S3 |
| 39177 | 550.4_Ex | AGAGA | | GGAGGGDRDGFRALC | | 620 | |
| 8 | on2_3rd_ | GTDGR | | VLVGGGGAVPGSFGP | | | |
| | 5 | | | DARPPHGAAGGWGSR | | | |
| | | | | GDRLGAGAGAGTDG | | | |
| | | | | RAEGPASTRGAAGIG | | | |
| | | | | GGGLGHGGGPGARPR | | | |
| | | | | ALAPATSAGGEPGAA | | | |
| | | | | GPRRGGRRERCLPPCR | | | |
| | | | | PRRGRPG | | | |

In some examples, a method includes, obtaining a sample, such as a first biological fluid sample from a subject that has or is believed to have a tumor and MSI is desired to be determined. The method includes applying the sample to an FSP array and detecting the binding of antibodies in the sample that bind with the FSP peptides in the array. This produces a baseline value of the antibody levels present. In particular, the binding of an antibody to an FSP peptide array creates a pattern of binding that can be associated with MSI, such as MSI-H, MSI-L or MSL-normal/average. MSI-H antibodies can create higher total binding to a set of peptides and a higher number of FSP with above average binding. These approaches can be used individually or in combination as a classifier of MSI-H status.

In some embodiments, the method includes providing a treatment to the subject that is need thereof. For example, in some embodiments, the disclosed method includes providing a course of treatment, such as treatment with a CPI therapy or not providing a CPI therapy as the tumor is not MSI-H.

In some embodiments, the method further includes obtaining a second biological sample from the subject at a desired time point, such as after a week, month, more than a month, year, or more than year from the treatment. The method includes applying the second sample to an FSP array and detecting the binding of antibodies in the sample that bind the FSP peptides in the array. The method includes comparing the antibody levels and/or isotypes, for example based on the peptide they bind, generated by the first sample and the second sample, thereby determining the MSI status. It is contemplated that the patient can be subsequently monitored as desired by the disclosed method for indication of MSI status as well as effectiveness of a vaccine. In some examples, the antibody profile changes indicating the tumor genetic make-up is changing. In some examples, the disclosed method is used for detection of epitope spreading. A FSP array is used to establish a baseline at time of treatment. If the therapy is killing tumor cells and they release new antigens, this can be detected on the arrays, for example as determined at a later time point.

ii. Production of Peptide Array

As disclosed herein, a FS peptide array is produced by translating genomic, but not natural protein coding, sequences into one or more peptides and then synthesizing the one or more peptides in situ on silica or glass wafers. These FSP sequences essentially replicated random sequences as they are not natural peptides. It is contemplated that the disclosed methods can be utilized to create frameshift peptides to generate Frameshift Arrays. In particular, the wafers are designed to make Frameshift Arrays by controlling the space between peptides to optimize cognate binding of the antibody.

The generated arrays are then developed as diagnostic platforms which can be used in the disclosed methods for determining MSI status. For example, a dilution of sera or blood or other antibody containing fluid is applied to the arrays and the antibodies detected with a secondary antibody. The bound antibodies create a signature of MSI status.

In some embodiments, methods of producing a set of peptides for detecting one or more antibodies that are associated with MSI includes identifying a signature peptide profile for MSI, such as a set of informative peptides correlated to MSI-H, MSI-L and/or MSI-average, and translating the signature peptide profile to one or more high affinity peptides for an antibody of interest, wherein the presence of the antibody of interest identifies MSI status and can be used to determine predicted responsiveness to CPI therapy.

In embodiments, identifying a signature FS peptide profile includes: translating non-coding genomic sequences, after excluding those encoding native proteins, into one or more peptides and then synthesizing the one or more peptides in situ on silica or glass wafers. In comparing MSI-H to MSS/MSI-L, MSI state antibodies specific for FSP can be identified. In embodiments, identifying differentially bound peptides includes identifying peptides on the peptide array that either bind less or more antibody in the profile of MSI-H versus MSS/MSI-L. The control can be any suitable control. In one embodiment, the control comprises MSI-average/normal biological sample, such as sera, contacted with an identical array under the same experimental conditions. The control can be values taken form such a control, such that the control and test need not be conducted at the same time. Comparison of the MSI-H to MSS/MSI-L profile to a normal control and identifying differentially bound peptides can be carried out via any suitable technique.

In IMs the binding of an antibody to a peptide array creates a pattern of binding that can be associated with a condition, such as MSI. The affinity of binding of an antibody to a peptide in the array can be mathematically associated with a condition. The binding pattern of an antibody to a plurality of different peptides of a peptide array can be mathematically associated with a condition. The avidity of binding of an antibody to a plurality of different peptides of a peptide array can be mathematically associated with a condition. This binding and avidity can comprise the interaction of an antibody in a biological sample with multiple, non-identical peptides in a peptide array. An avidity of binding of an antibody with multiple, non-identical peptides in a peptide array can determine an association constant of the antibody to the peptide array. In some embodiments, the concentration of an antibody in a sample contributes to an avidity of binding to a peptide array, for example, by trapping a critical number or antibodies in the array and allowing for rapid rebinding of an antibody to an array.

The avidity of binding of an antibody to a peptide array can be determined by a combination of multiple bond interaction. A cross-reactivity of an antibody to multiple peptides in a peptide array can contribute to an avidity of binding. In some embodiments, an antibody can recognize an epitope of about 3 amino acids, about 4 amino acids, about 5 amino acids, about 6 amino acids, about 7 amino acids, about 8 amino acids, about 9 amino acids, about 10 amino acids, about 11 amino acids, about 12 amino acids, about 13 amino acids, about 14 amino acids, about 15 amino acids, about 16 amino acids, about 17 amino acids, about 18 amino acids, about 19 amino acids or about 20 amino acids. In some embodiments, a sequence of about 5 amino acids dominates a binding energy of an antibody to a peptide.

Off-target binding, and/or avidity, of an antibody to a peptide within a peptide array, for example, effectively compresses binding affinities that span femtomolar (fM) to micromolar (μM) dissociation constants into a range that can be quantitatively measured using only 3 logs of dynamic range. Avidity depends on the effective trapping of the antibody because the peptides are close enough together. An antibody can bind to a plurality of peptides in the array with association constants of 10³M⁻¹ or higher. An antibody can bind to a plurality of peptides in the array with association constants ranging from 10³ to 10⁶ M⁻¹, 2×10³ M⁻¹ to 10⁶ M⁻¹, and/or association constants ranging from 104 M⁻¹ to 10⁶M⁻¹. An antibody can bind to a plurality of peptides in the array with a dissociation constant of about 1 fM, about 2 fM, about 3 fM, about 4 fM, about 5 fM, about 6 fM, about 7 fM, about 8 fM, about 9 fM, about 10 fM, about 20 fM, about 30 fM, about 40 fM, about 50 fM, about 60 fM, about 70 fM, about 80 fM, about 90 fM, about 100 fM, about 200 fM, about 300 fM, about 400 fM, about 500 fM, about 600 fM, about 700 fM, about 800 fM, about 900 fM, about 1 picomolar (pM), about 2 pM, about 3 pM, about 4 pM, about 5 pM, about 6 pM, about 7 pM, about 8 pM, about 9 pM, about 10 pM, about 20 pM, about 30 pM, about 40 pM, about 50 pM, about 60 pM, about 7 pM, about 80 pM, about 90 pM, about 100 pM, about 200 pM, about 300 pM, about 400 pM, about 500 pM, about 600 pM, about 700 pM, about 800 pM, about 900 pM, about 1 nanomolar (nM), about 2 nM, about 3 nM, about 4 nM, about 5 nM, about 6 nM, about 7 nM, about 8 nM, about 9 nM, about 10 nM, about 20 nM, about 30 nM, about 40 nM, about 50 nm, about 60 nM, about 70 nM, about 80 nM, about 90 nM, about 100 nM, about 200 nM, about 300 nM, about 400 nM, about 500 nM, about 600 nM, about 700 nM, about 800 nM, about 900 nM, about 1 μM, about 2 μM, about 3 μM, about 4 μM, about 5 μM, about 6 μM, about 7 μM, about 8 μM, about 9 μM, about 10 μM, about 20 μM, about 30 μM, about 40 μM, about 50 μM, about 60 μM, about 70 μM, about 80 μM, about 90 μM, or about 100 μM.

An antibody can bind to a plurality of peptides in the array with a dissociation constant of at least 1 fM, at least 2 fM, at least 3 fM, at least 4 fM, at least 5 fM, at least 6 fM, at least 7 fM, at least 8 fM, at least 9 fM, at least 10 fM, at least 20 fM, at least 30 fM, at least 40 fM, at least 50 fM, at least 60 fM, at least 70 fM, at least 80 fM, at least 90 fM, at least 100 fM, at least 200 fM, at least 300 fM, at least 400 fM, at least 500 fM, at least 600 fM, at least 700 fM, at least 800 fM, at least 900 fM, at least 1 picomolar (pM), at least 2 pM, at least 3 pM, at least 4 pM, at least 5 pM, at least 6 pM, at least 7 pM, at least 8 pM, at least 9 pM, at least 10 pM, at least 20 pM, at least 30 pM, at least 40 pM, at least 50 pM, at least 60 pM, at least 7 pM, at least 80 pM, at least 90 pM, at least 100 pM, at least 200 pM, at least 300 pM, at least 400 pM, at least 500 pM, at least 600 pM, at least 700 pM, at least 800 pM, at least 900 pM, at least 1 nanomolar (nM), at least 2 nM, at least 3 nM, at least 4 nM, at least 5 nM, at least 6 nM, at least 7 nM, at least 8 nM, at least 9 nM, at least 10 nM, at least 20 nM, at least 30 nM, at least 40 nM, at least 50 nm, at least 60 nM, at least 70 nM, at least 80 nM, at least 90 nM, at least 100 nM, at least 200 nM, at least 300 nM, at least 400 nM, at least 500 nM, at least 600 nM, at least 700 nM, at least 800 nM, at least 900 nM, at least 1 μM, at least 2 μM, at least 3 μM, at least 4 μM, at least 5 μM, at least 6 μM, at least 7 μM, at least 8 μM, at least 9 μM, at least 10 μM, at least 20 μM, at least 30 μM, at least 40 μM, at least 50 μM, at least 60 μM, at least 70 μM, at least 80 μM, at least 90 μM, or about 100 μM.

A dynamic range of binding of an antibody from a biological sample to a peptide microarray can be described as the ratio between the largest and smallest value of a detected signal of binding. A signal of binding can be, for example, a fluorescent signal detected with a secondary antibody. Traditional assays are limited by pre-determined and narrow dynamic ranges of binding. The methods and arrays of the disclosure can detected a broad dynamic range of antibody binding to the peptides in the array. In some embodiments, a broad dynamic range of antibody binding can be detected on a logarithmic scale. In some embodiments, the methods and arrays of the disclosure allow the detection of a pattern of binding of a plurality of antibodies to an array using up to 2 logs of dynamic range, up to 3 logs of dynamic range, up to 4 logs of dynamic range or up to 5 logs of dynamic range.

As used herein, the term “substrate” refers to any type of solid support to which the peptides are immobilized. Examples of substrates include, but are not limited to, microarrays; beads; columns; optical fibers; wipes; nitrocellulose; nylon; glass; quartz; diazotized membranes (paper or nylon); silicones; polyformaldehyde; cellulose; cellulose acetate; paper; ceramics; metals; metalloids; semiconductive materials; coated beads; magnetic particles; plastics such as polyethylene, polypropylene, and polystyrene; gel-forming materials; silicates; agarose; polyacrylamides; methylmethracrylate polymers; sol gels; porous polymer hydrogels; nanostructured surfaces; nanotubes (such as carbon nanotubes); and nanoparticles (such as gold nanoparticles or quantum dots). When bound to a substrate, the peptides can be directly linked to the support, or attached to the surface via a linker. Thus, the solid substrate and/or the peptides can be derivatized using methods known in the art to facilitate binding of the peptides to the solid support, so long as the derivitization does not eliminate detection of binding between the peptides and an antibody. In the present disclosure, the peptides need to be close enough to each other, such as within 3 nm or less to facilitate avidity.

Other molecules, such as reference or control molecules, can be optionally immobilized on the substrate as well. Methods for immobilizing various types of molecules on a variety of substrates are well known to those of skill in the art. A wide variety of materials can be used for the solid surface. A variety of different materials can be used to prepare the support to obtain various properties. For example, proteins (e.g., bovine serum albumin) or mixtures of macromolecules (e.g., Denhardt's solution) can be used to minimize non-specific binding, simplify covalent conjugation, and/or enhance signal detection.

The peptide arrays can be contacted with a biological sample under any suitable conditions to promote binding of antibodies in the biological sample to peptides immobilized on the array. Thus, the disclosed methods are not limited by any specific type of binding conditions employed. Such conditions will vary depending on the array being used, the type of substrate, the density of the peptides arrayed on the substrate, desired stringency of the binding interaction, and nature of the competing materials in the binding solution. In some embodiments, the conditions comprise a step to remove unbound antibodies from the addressable array. Determining the need for such a step, and appropriate conditions for such a step, are well within the level of skill in the art.

Similarly, any suitable detection technique can be used in the disclosed methods detecting binding of antibodies in the biological sample to peptides on the array to generate a disease immune profile; In one embodiment, any type of detectable label can be used to label antibodies on the array, including but not limited to radioisotope labels, fluorescent labels, luminescent labels, and electrochemical labels (i.e.: ligand labels with different electrode mid-point potential, where detection comprises detecting electric potential of the label). Alternatively, bound antibodies can be detected, for example, using a detectably labeled secondary antibody. Methods that directly the bound antibodies, such as plasmon surface resonance, can also be used.

A peptide array can comprise a plurality of different peptides patterns a surface. A peptide array can comprise, for example, a single, a duplicate, a triplicate, a quadruplicate, a quintuplicate, a sextuplicate, a septuplicate, an octuplicate, a nonuplicate, and/or a decuplicate replicate of the different pluralities of peptides and/or molecules. In some embodiments, pluralities of different peptides are spotted or synthesized in replica on the surface of a peptide array. A peptide array can, for example, comprise a plurality of peptides homogenously distributed on the array. A peptide array can, for example, comprise a plurality of peptides heterogeneously distributed on the array.

An inter-peptide acid distance in a peptide array is the distance between each peptide in a peptide microarray. An inter-peptide distance can contribute to an off-target binding and/or to an avidity of binding of an antibody to an array. An intra-amino acid difference can be about 0.5 nm, about 1 nm, about 1 nm, 1.1 nm, about 1.2 nm, about 1.3 nm, about 1.4 nm, about 1.5 nm, about 1.6 nm, about 1.7 nm, about 1.8 nm, about 1.9 nm, about 2 nm, about 2.1 nm, about 2.2 nm, about 2.3 nm, about 2.4 nm, about 2.5 nm, about 2.6 nm, about 2.7 nm, about 2.8 nm, about 2.9 nm, about 3 nm. In some embodiments, the inter-peptide difference is between about 0.5 nm and about 3 nm. For IMS arrays the distance is generally 1-3 nm.

An inter-peptide difference can be at least 0.5 nm, at least 1 nm, at least 1 nm, at least 1.1 nm, at least 1.2 nm, at least 1.3 nm, at least 1.4 nm, at least 1.5 nm, at least 1.6 nm, at least 1.7 nm, at least 1.8 nm, at least 1.9 nm, at least 2 nm, at least 2.1 nm, at least 2.2 nm, at least 2.3 nm, at least 2.4 nm, at least 2.5 nm, at least 2.6 nm, at least 2.7 nm, at least 2.8 nm, at least 2.9 nm, at least 3 nm.

An inter-peptide difference can be not more than 3 nm, not more than 3.1 nm, not more than 3.2 nm, not more than 3.3 nm, not more than 3.4 nm, not more than 3.5 nm, not more than 3.6 nm, not more than 3.7 nm, not more than 3.8 nm, not more than 3.9 nm, not more than 4 nm, not more than 4.1 nm, not more than 4.2 nm, not more than 4.3 nm, not more than 4.4 nm, not more than 4.5 nm, not more than 4.6 nm, not more than 4.7 nm, not more than 4.8 nm, not more than 4.9 nm, not more than 5 nm, not more than 5.1 nm, not more than 5.2 nm, not more than 5.3 nm, not more than 5.4 nm, not more than 5.5 nm, not more than 5.6 nm, not more than 5.7 nm, not more than 5.8 nm, not more than 5.9 nm, and/or not more than 6 nm. In some embodiments, the intra-amino acid distance is not more than 6 nanometers (nm). For FSP arrays the inter-peptide distance is generally at least 3 nm and not more than 6 nm.

A peptide array can comprise a plurality of different peptides patterns a surface. A peptide array can comprise, for example, a single, a duplicate, a triplicate, a quadruplicate, a quintuplicate, a sextuplicate, a septuplicate, an octuplicate, a nonuplicate, and/or a decuplicate replicate of the different pluralities of peptides and/or molecules. In some embodiments, pluralities of different peptides are spotted in replica on the surface of a peptide array. A peptide array can, for example, comprise a plurality of peptides homogenously distributed on the array. A peptide array can, for example, comprise a plurality of peptides heterogeneously distributed on the array.

A peptide can be “spotted” in a peptide array. A peptide spot can have various geometric shapes, for example, a peptide spot can be round, square, rectangular, and/or triangular. A peptide spot can have a plurality of diameters. Non-limiting examples of peptide spot diameters are about 3 μm to about 8 μm, about 3 to about 10 mm, about 5 to about 10 mm, about 10 μm to about 20 μm, about 30 μm, about 40 μm, about 50 μm, about 60 μm, about 70 μm, about 80 μm, about 90 μm, about 100 μm, about 110 μm, about 120 μm, about 130 μm, about 140 μm, about 150 μm, about 160 μm, about 170 μm, about 180 μm, about 190 μm, about 200 μm, about 210 μm, about 220 μm, about 230 μm, about 240 μm, and/or about 250 μm.

A peptide array can comprise a number of different peptides. In some embodiments, a peptide array comprises about 10 peptides, about 50 peptides, about 100 peptides, about 200 peptides, about 300 peptides, about 400 peptides, about 500 peptides, about 750 peptides, about 1000 peptides, about 1250 peptides, about 1500 peptides, about 1750 peptides, about 2,000 peptides; about 2,250 peptides; about 2,500 peptides; about 2,750 peptides; about 3,000 peptides; about 3,250 peptides; about 3,500 peptides; about 3,750 peptides; about 4,000 peptides; about 4,250 peptides; about 4,500 peptides; about 4,750 peptides; about 5,000 peptides; about 5,250 peptides; about 5,500 peptides; about 5,750 peptides; about 6,000 peptides; about 6,250 peptides; about 6,500 peptides; about 7,500 peptides; about 7,725 peptides 8,000 peptides; about 8,250 peptides; about 8,500 peptides; about 8,750 peptides; about 9,000 peptides; about 9,250 peptides; about 10,000 peptides; about 10,250 peptides; about 10,500 peptides; about 10,750 peptides; about 11,000 peptides; about 11,250 peptides; about 11,500 peptides; about 11,750 peptides; about 12,000 peptides; about 12,250 peptides; about 12,500 peptides; about 12,750 peptides; about 13,000 peptides; about 13,250 peptides; about 13,500 peptides; about 13,750 peptides; about 14,000 peptides; about 14,250 peptides; about 14,500 peptides; about 14,750 peptides; about 15,000 peptides; about 15,250 peptides; about 15,500 peptides; about 15,750 peptides; about 16,000 peptides; about 16,250 peptides; about 16,500 peptides; about 16,750 peptides; about 17,000 peptides; about 17,250 peptides; about 17,500 peptides; about 17,750 peptides; about 18,000 peptides; about 18,250 peptides; about 18,500 peptides; about 18,750 peptides; about 19,000 peptides; about 19,250 peptides; about 19,500 peptides; about 19,750 peptides; about 20,000 peptides; about 20,250 peptides; about 20,500 peptides; about 20,750 peptides; about 21,000 peptides; about 21,250 peptides; about 21,500 peptides; about 21,750 peptides; about 22,000 peptides; about 22,250 peptides; about 22,500 peptides; about 22,750 peptides; about 23,000 peptides; about 23,250 peptides; about 23,500 peptides; about 23,750 peptides; about 24,000 peptides; about 24,250 peptides; about 24,500 peptides; about 24,750 peptides; about 25,000 peptides; about 25,250 peptides; about 25,500 peptides; about 25,750 peptides; and/or about 30,000 peptides.

In some embodiments, a peptide array used in the methods and devices herein comprises more than 30,000 peptides. In some embodiments, a peptide array used in a method of determining MSI comprises about 330,000 peptides. In some embodiments the array comprise about 30,000 peptides; about 35,000 peptides; about 40,000 peptides; about 45,000 peptides; about 50,000 peptides; about 55,000 peptides; about 60,000 peptides; about 65,000 peptides; about 70,000 peptides; about 75,000 peptides; about 80,000 peptides; about 85,000 peptides; about 90,000 peptides; about 95,000 peptides; about 100,000 peptides; about 105,000 peptides; about 110,000 peptides; about 115,000 peptides; about 120,000 peptides; about 125,000 peptides; about 130,000 peptides; about 135,000 peptides; about 140,000 peptides; about 145,000 peptides; about 150,000 peptides; about 155,000 peptides; about 160,000 peptides; about 165,000 peptides; about 170,000 peptides; about 175,000 peptides; about 180,000 peptides; about 185,000 peptides; about 190,000 peptides; about 195,000 peptides; about 200,000 peptides; about 210,000 peptides; about 215,000 peptides; about 220,000 peptides; about 225,000 peptides; about 230,000 peptides; about 240,000 peptides; about 245,000 peptides; about 250,000 peptides; about 255,000 peptides; about 260,000 peptides; about 265,000 peptides; about 270,000 peptides; about 275,000 peptides; about 280,000 peptides; about 285,000 peptides; about 290,000 peptides; about 295,000 peptides; about 300,000 peptides; about 305,000 peptides; about 310,000 peptides; about 315,000 peptides; about 320,000 peptides; about 325,000 peptides; about 330,000 peptides; about 335,000 peptides; about 340,000 peptides; about 345,000 peptides; about 350,000 peptides; about 360,000 peptides; about 370,000 peptides; about 380,000 peptides; about 390,000 peptides; about 400,000 peptides; about 405,000 peptides; about 408,000 peptides and/or about 410,000 peptides. In some embodiments, a peptide array used in a method of determining MSI comprises more than 330,000 peptides, more than 350,000 peptides, more than 400,000 peptides such as between 350,000 and 410,000 peptides, or 400,000 and 410,000 peptides.

A peptide array can comprise a number of different peptides. In some embodiments, a peptide array for determining MSI comprises at least 100 FS peptides, at least 250 FS peptides, at least 1,000 FS peptides, at least 2,000 FS peptides; at least 3,000 FS peptides; at least 4,000 FS peptides; at least 5,000 FS peptides; at least 6,000 FS peptides; at least 7,000 FS peptides; at least 8,000 FS peptides; at least 9,000 FS peptides; at least 10,000 FS peptides; at least 11,000 FS peptides; at least 12,000 FS peptides; at least 13,000 FS peptides; at least 14,000 FS peptides; at least 15,000 FS peptides; at least 16,000 FS peptides; at least 17,000 FS peptides; at least 18,000 FS peptides; at least 19,000 FS peptides; at least 20,000 FS peptides; at least 21,000 FS peptides; at least 22,000 FS peptides; at least 23,000 FS peptides; at least 24,000 FS peptides; at least 25,000 FS peptides; at least 30,000 FS peptides; at least 40,000 FS peptides; at least 50,000 FS peptides; at least 60,000 FS peptides; at least 70,000 FS peptides; at least 80,000 FS peptides; at least 90,000 FS peptides; at least 100,000 FS peptides; at least 110,000 FS peptides; at least 120,000 FS peptides; at least 130,000 FS peptides; at least 140,000 FS peptides; at least 150,000 FS peptides; at least 160,000 FS peptides; at least about 170,000 FS peptides, at least 180,000 FS peptides; at least 190,000 FS peptides; at least 200,000 FS peptides; at least 210,000 FS peptides; at least 220,000 FS peptides; at least 230,000 FS peptides; at least 240,000 FS peptides; at least 250,000 FS peptides; at least 260,000 FS peptides; at least 270,000 FS peptides; at least 280,000 peptides; at least 290,000 FS peptides; at least 300,000 FS peptides; at least 310,000 FS peptides; at least 320,000 FS peptides; at least 330,000 FS peptides; at least 340,000 FS peptides; at least 350,000 FS peptides. In some embodiments, a peptide array used in a method of determining MSI comprises at least 330,000 FS peptides. In some embodiments, 400,000 FS peptides are used on the array.

A peptide can be physically tethered to a peptide array by a linker molecule. The N- or the C-terminus of the peptide can be attached to a linker molecule. A linker molecule can be, for example, a functional plurality or molecule present on the surface of an array, such as an imide functional group, an amine functional group, a hydroxyl functional group, a carboxyl functional group, an aldehyde functional group, and/or a sulfhydryl functional group. A linker molecule can be, for example, a polymer. In some embodiments the linker is maleimide. In some embodiments the linker is a glycine-serine-cysteine (GSC) or glycine-glycine-cysteine (GGC) linker. In some embodiments, the linker consists of a polypeptide of various lengths or compositions. In some cases, the linker is polyethylene glycol of different lengths. In yet other cases, the linker is hydroxymethyl benzoic acid, 4-hydroxy-2-methoxy benzaldehyde, 4-sulfamoyl benzoic acid, or other suitable for attaching a peptide to the solid substrate.

A surface of a peptide array can comprise a plurality of different materials. A surface of a peptide array can be, for example, glass. Non-limiting examples of materials that can comprise a surface of a peptide array include glass, functionalized glass, silicon, germanium, gallium arsenide, gallium phosphide, silicon dioxide, sodium oxide, silicon nitrade, nitrocellulose, nylon, polytetraflouroethylene, polyvinylidendiflouride, polystyrene, polycarbonate, methacrylates, or combinations thereof.

A surface of a peptide array can be flat, concave, or convex. A surface of a peptide array can be homogeneous and a surface of an array can be heterogeneous. In some embodiments, the surface of a peptide array is flat.

A surface of a peptide array can be coated with a coating. A coating can, for example, improve the adhesion capacity of an array. A coating can, for example, reduce background adhesion of a biological sample to an array. In some embodiments, a peptide array of comprises a glass slide with an aminosilane-coating.

A peptide array can have a plurality of dimensions. A peptide array can be a peptide microarray.

Binding interactions between components of a sample and an array can be detected in a variety of formats. In some formats, components of the samples are labeled. The label can be a radioisotype or dye among others. The label can be supplied either by administering the label to a patient before obtaining a sample or by linking the label to the sample or selective component(s) thereof.

Binding interactions can also be detected using a secondary detection reagent, such as an antibody. For example, binding of antibodies in a sample to an array can be detected using a secondary antibody specific for the isotype of an antibody (e.g., IgG (including any of the subtypes, such as IgG1, IgG2, IgG3 and IgG4), IgA, IgM). The secondary antibody is usually labeled and can bind to all antibodies in the sample being analyzed of a particular isotype. Different secondary antibodies can be used having different isotype specificities. Although there is often substantial overlap in compounds bound by antibodies of different isotypes in the same sample, there are also differences in profile.

Binding interactions can also be detected using label-free methods, such as surface plasmon resonance (SPR) and mass spectrometry. SPR can provide a measure of dissociation constants, and dissociation rates. The A-100 Biocore/GE instrument, for example, is suitable for this type of analysis. FLEXchips can be used to analyze up to 400 binding reactions on the same support.

An array containing FS peptides generated in tumors can be produced and antibody reactivity is determined using an assay for antibody binding to a peptide. In some embodiments, FS peptides for antibody detection are bound to a substrate, such as a plate, a glass slide, a bead, or other substrate. Assays for antibody binding include but are not limited to ELISA, radioimmunoassay, western blot, surface plasmon resonance, immunostaining, immunoprecipitation, mass spectrometry, phage display, flow cytometry, cytometric bead array, immunohistochemistry, high density array, microarray, and combinations thereof.

To develop antibodies for MSI regardless of the particular subject, a smaller subset of neo-antigens (“frameshift neo-antigens”) can be utilized. Because the number of frameshift (FS) neo-antigens is much smaller than the total of all possible neo-antigens it is possible to produce arrays of these FS neo-antigens and screen these FS array against a set of biological samples obtained from subjects having a particular cancer and/or tumor type of interest, such as MSI-H cancer and/or tumor. The inventors have discovered that tumors make indel mutations in most microsatellites and that mis-splicing is also recurrent at the same genes in tumors. The FS peptides are produced by insertion and deletion mutations (indels) occurring in microsatellite regions or by mis-splicing of RNA. There are approximately 10,000 potential FS peptides transcribed from microsatellites in exons and approximately 600,000 potential FS from mis-splicing. The number of FS to search can be limited by restricting them to ones that are at least 8 amino acids long and/or in oncogenes, essential genes and or highly expressed genes. These genes would be more difficult for a tumor to evolve away from. Therefore, it is only necessary to screen a limited number of approximately 100 to a few thousand FS peptides to determine the immunogenic components for a specific type of cancer and/or tumor, such as an MSI-H. In some embodiments, an array with sequences set forth in Tables 1, 2 and/or 3 is used to determine MSI status.

In some embodiments, the biological sample comprises or is selected from the group consisting of blood, plasma, serum, thymus, bone marrow, spleen, lymph node, bronchoalveolar lavage, breast, central nervous system, cerebrospinal fluid, eye, tears, gastrointestinal tract, saliva, feces, urine, heart, kidney, liver, lung, muscle, pancreas, peripheral nervous system, saliva, skin, thyroid, trachea, and tumor. In some embodiments, the biological sample is blood, serum, plasma, or saliva. In some embodiments, the biological sample comprises an antibody. In some embodiments, the biological sample comprises an antibody and cells selected from B cells, T cells, CD4+ T cells, CD8+ T cells, Th17 cells, or combinations thereof. In some embodiments, the biological sample comprises an antibody. In some embodiments, all the biological samples tested are the same type. In other embodiments, the biological samples are different types, such as different types of samples listed above.

In some embodiments, the cancer and/or tumor type comprises bladder cancer, lung cancer, colon cancer, endometrial cancer, stomach cancer, kidney cancer, melanoma, head cancer, neck cancer, Hodgkin's lymphoma and solid tumors. In some embodiments, the cancer and/or tumor type comprises a MSI-H cancer and/or tumor. In some examples, the cancer and/or tumor type comprises at least one of Acanthoma, Acinic cell carcinoma, Acoustic neuroma, Acral lentiginous melanoma, Acrospiroma, Acute eosinophilic leukemia, Acute lymphoblastic leukemia, Acute megakaryoblastic leukemia, Acute monocytic leukemia, Acute myeloblastic leukemia with maturation, Acute myeloid dendritic cell leukemia, Acute myeloid leukemia, Acute promyelocytic leukemia, Adamantinoma, Adenocarcinoma, Adenoid cystic carcinoma, Adenoma, Adenomatoid odontogenic tumor, Adrenocortical carcinoma, Adult T-cell leukemia, Aggressive NK-cell leukemia, AIDS-Related Cancers, AIDS-related lymphoma, Alveolar soft part sarcoma, Ameloblastic fibroma, Anal cancer, Anaplastic large cell lymphoma, Anaplastic thyroid cancer, Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone Cancer, Bone tumor, Brain Stem Glioma, Brain Tumor, Breast Cancer, Brenner tumor, Bronchial Tumor, Bronchioloalveolar carcinoma, Brown tumor, Burkitt's lymphoma, Cancer of Unknown Primary Site, Carcinoid Tumor, Carcinoma, Carcinoma in situ, Carcinoma of the penis, Carcinoma of Unknown Primary Site, Carcinosarcoma, Castleman's Disease, Central Nervous System Embryonal Tumor, Cerebellar Astrocytoma, Cerebral Astrocytoma, Cervical Cancer, Cholangiocarcinoma, Chondroma, Chondrosarcoma, Chordoma, Choriocarcinoma, Choroid plexus papilloma, Chronic Lymphocytic Leukemia, Chronic monocytic leukemia, Chronic myelogenous leukemia, Chronic Myeloproliferative Disorder, Chronic neutrophilic leukemia, Clear-cell tumor, Colon Cancer, Colorectal cancer, Craniopharyngioma, Cutaneous T-cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial Uterine Cancer, Endometrioid tumor, Enteropathy-associated T-cell lymphoma, Ependymoblastoma, Ependymoma, Epithelioid sarcoma, Erythroleukemia, Esophageal cancer, Esthesioneuroblastoma, Ewing Family of Tumor, Ewing Family Sarcoma, Ewing's sarcoma, Extracranial Germ Cell Tumor, Extragonadal Germ Cell Tumor, Extrahepatic Bile Duct Cancer, Extramammary Paget's disease, Fallopian tube cancer, Fetus in fetu, Fibroma, Fibrosarcoma, Follicular lymphoma, Follicular thyroid cancer, Gallbladder Cancer, Gallbladder cancer, Ganglioglioma, Ganglioneuroma, Gastric Cancer, Gastric lymphoma, Gastrointestinal cancer, Gastrointestinal Carcinoid Tumor, Gastrointestinal Stromal Tumor, Gastrointestinal stromal tumor, Germ cell tumor, Germinoma, Gestational choriocarcinoma, Gestational Trophoblastic Tumor, Giant cell tumor of bone, Glioblastoma multiforme, Glioma, Gliomatosis cerebri, Glomus tumor, Glucagonoma, Gonadoblastoma, Granulosa cell tumor, Hairy Cell Leukemia, Hairy cell leukemia, Head and Neck Cancer, Head and neck cancer, Heart cancer, Hemangioblastoma, Hemangiopericytoma, Hemangiosarcoma, Hematological malignancy, Hepatocellular carcinoma, Hepatosplenic T-cell lymphoma, Hereditary breast-ovarian cancer syndrome, Hodgkin Lymphoma, Hodgkin's lymphoma, Hypopharyngeal Cancer, Hypothalamic Glioma, Inflammatory breast cancer, Intraocular Melanoma, Islet cell carcinoma, Islet Cell Tumor, Juvenile myelomonocytic leukemia, Kaposi Sarcoma, Kaposi's sarcoma, Kidney Cancer, Klatskin tumor, Krukenberg tumor, Laryngeal Cancer, Laryngeal cancer, Lentigo maligna melanoma, Leukemia, Lip and Oral Cavity Cancer, Liposarcoma, Lung cancer, Luteoma, Lymphangioma, Lymphangiosarcoma, Lymphoepithelioma, Lymphoid leukemia, Lymphoma, Macroglobulinemia, Malignant Fibrous Histiocytoma, Malignant fibrous histiocytoma, Malignant Fibrous Histiocytoma of Bone, Malignant Glioma, Malignant Mesothelioma, Malignant peripheral nerve sheath tumor, Malignant rhabdoid tumor, Malignant triton tumor, MALT lymphoma, Mantle cell lymphoma, Mast cell leukemia, Mediastinal germ cell tumor, Mediastinal tumor, Medullary thyroid cancer, Medulloblastoma, Medulloepithelioma, Melanoma, Meningioma, Merkel Cell Carcinoma, Mesothelioma, Metastatic Squamous Neck Cancer with Occult Primary, Metastatic urothelial carcinoma, Mixed Mullerian tumor, Monocytic leukemia, Mouth Cancer, Mucinous tumor, Multiple Endocrine Neoplasia Syndrome, Multiple myeloma, Mycosis Fungoides, Myelodysplastic Disease, Myelodysplastic Syndromes, Myeloid leukemia, Myeloid sarcoma, Myeloproliferative Disease, Myxoma, Nasal Cavity Cancer, Nasopharyngeal Cancer, Nasopharyngeal carcinoma, Neoplasm, Neurinoma, Neuroblastoma, Neurofibroma, Neuroma, Nodular melanoma, Non-Hodgkin lymphoma, Nonmelanoma Skin Cancer, Non-Small Cell Lung Cancer, Ocular oncology, Oligoastrocytoma, Oligodendroglioma, Oncocytoma, Optic nerve sheath meningioma, Oral cancer, Oropharyngeal Cancer, Osteosarcoma, Ovarian cancer, Ovarian Epithelial Cancer, Ovarian Germ Cell Tumor, Ovarian Low Malignant Potential Tumor, Paget's disease of the breast, Pancoast tumor, Pancreatic cancer, Papillary thyroid cancer, Papillomatosis, Paraganglioma, Paranasal Sinus Cancer, Parathyroid Cancer, Penile Cancer, Perivascular epithelioid cell tumor, Pharyngeal Cancer, Pheochromocytoma, Pineal Parenchymal Tumor of Intermediate Differentiation, Pineoblastoma, Pituicytoma, Pituitary adenoma, Pituitary tumor, Plasma Cell Neoplasm, Pleuropulmonary blastoma, Polyembryoma, Precursor T-lymphoblastic lymphoma, Primary central nervous system lymphoma, Primary effusion lymphoma, Primary Hepatocellular Cancer, Primary Liver Cancer, Primary peritoneal cancer, Primitive neuroectodermal tumor, Prostate cancer, Pseudomyxoma peritonei, Rectal Cancer, Renal cell carcinoma, Respiratory Tract Carcinoma Involving the NUT Gene on Chromosome 15, Retinoblastoma, Rhabdomyoma, Rhabdomyosarcoma, Richter's transformation, Sacrococcygeal teratoma, Salivary Gland Cancer, Sarcoma, Schwannomatosis, Sebaceous gland carcinoma, Secondary neoplasm, Seminoma, Serous tumor, Sertoli-Leydig cell tumor, Sex cord-stromal tumor, Sezary Syndrome, Signet ring cell carcinoma, Skin Cancer, Small blue round cell tumor, Small cell carcinoma, Small Cell Lung Cancer, Small cell lymphoma, Small intestine cancer, Soft tissue sarcoma, Somatostatinoma, Soot wart, Spinal Cord Tumor, Spinal tumor, Splenic marginal zone lymphoma, Squamous cell carcinoma, Stomach cancer, Superficial spreading melanoma, Supratentorial Primitive Neuroectodermal Tumor, Surface epithelial-stromal tumor, Synovial sarcoma, T-cell acute lymphoblastic leukemia, T-cell large granular lymphocyte leukemia, T-cell leukemia, T-cell lymphoma, T-cell prolymphocytic leukemia, Teratoma, Terminal lymphatic cancer, Testicular cancer, Thecoma, Throat Cancer, Thymic Carcinoma, Thymoma, Thyroid cancer, Transitional Cell Cancer of Renal Pelvis and Ureter, Transitional cell carcinoma, Urachal cancer, Urethral cancer, Urogenital neoplasm, Uterine sarcoma, Uveal melanoma, Vaginal Cancer, Verner Morrison syndrome, Verrucous carcinoma, Visual Pathway Glioma, Vulvar Cancer, Waldenstrom's macroglobulinemia, Warthin's tumor, or Wilms' tumor. Thus, using the methods disclosed herein cancer treatments can be determine and/or produced for any of the listed cancer and/or tumor types as well as any that are not listed.

In some embodiments, disclosed herein are array platforms that allow for development of peptides suitable for detecting antibodies to a specific cancer and/or tumor type, such as on that is MSI-H. The array platforms comprise a plurality of subject features on the surface of the array, for example in addressable locations. Each feature typically comprises a plurality of subject peptides synthesized in situ on the surface of the array, wherein the molecules are identical within a feature, but the sequence or identity of the molecules differ between features. Such array molecules include the synthesis of large synthetic peptide arrays.

The peptide arrays can include control sequences that match epitopes of well characterized monoclonal antibodies (mAbs). Binding patterns to control sequences and to library peptides can be measured to qualify the arrays and the assay process. Additionally, inter wafer signal precision can be determined by testing sample replicates e.g. plasma samples, on arrays from different wafers and calculating the coefficients of variation (CV) for all library peptides. Precision of the measurements of binding signals can be determined as an aggregate of the inter-array, inter-slide, inter-wafer and inter-day variations made on arrays synthesized on wafers of the same batch (within wafer batches). Additionally, precision of measurements can be determined for arrays on wafers of different batches (between wafer batches). In some embodiments, measurements of binding signals can be made within and/or between wafer batches with a precision varying less than 5%, less than 10%, less than 15%, less than 20%, less than 25%, or less than 30%.

The technologies disclosed herein include a photolithographic array synthesis platform that merges semiconductor manufacturing processes and combinatorial chemical synthesis to produce array-based libraries on silicon wafers. By utilizing the tremendous advancements in photolithographic feature patterning, the array synthesis platform is highly-scalable and capable of producing combinatorial peptide libraries with 40 million features on an 8-inch wafer. Photolithographic array synthesis is performed using semiconductor wafer production equipment in a class 10,000 cleanroom to achieve high reproducibility. When the wafer is diced into standard microscope slide dimensions, each slide contains more than 3 million distinct chemical entities.

In some embodiments, arrays with peptide libraries produced by photolithographic technologies disclosed herein are used for immune-based assays. Using a subject's, or multiple subjects, antibody repertoire from a biological sample bound to the arrays, a fluorescence binding profile image of the bound array provides sufficient information to classify which peptides are reactive with an antibody from the subject, such as one associated with MSI.

Platforms disclosed herein comprise a selection of frameshift peptides disclosed herein, such as peptides resulting from an insertion or deletion error in transcription of an mRNA or peptides resulting from a splicing error such as a trans-splicing error or a cis-splicing error. In some embodiments, platforms herein comprise frameshift peptides comprise peptides having a sequence selected from all MS FS or MS FS from oncogenes, essential genes, or highly expressed genes associated with MSI, such as at least one or more provided in Tables 1, 2 and/or 3.

In some embodiments, the array is a wafer-based, photolithographic, in situ peptide array produced using reusable masks and automation to obtain arrays of scalable numbers of combinatorial sequence peptides. In some embodiments, the peptide array comprises about 20, about 23, about 30, about 40, about 50, about 60, about 70, about 80, about 90, about 100, about 500, about 1000, about 2000, about 3000, about 4000, about 5,000, about 6000, about 7000, about 8000, about 9000, about 10,000, about 15,000, about 20,000, about 30,000, about 40,000, about 50,000, about 100,000, about 200,000, about 300,000, about 400,000, about 500,000, or more FS peptides having different sequences. Multiple copies of each of the different sequence peptides can be situated on the wafer at addressable locations known as features.

In some embodiments, the array is a glass slide or nitrocellulose membrane having in vitro synthesized peptides spotted in a predetermined pattern and screened for binding of antibodies in a biological sample, such as obtained from one or more subjects.

In some embodiments, detection of antibody binding on a peptide array poses some challenges that can be addressed by the technologies disclosed herein. Accordingly, in some embodiments, the arrays and methods disclosed herein utilize specific coatings and functional group densities on the surface of the array that can tune the desired properties necessary for performing assays. For example, non-specific antibody binding on a peptide array may be minimized by coating the silicon surface with a moderately hydrophilic monolayer polyethylene glycol (PEG), polyvinyl alcohol, carboxymethyl dextran, and combinations thereof. In some embodiments, the hydrophilic monolayer is homogeneous. Second, synthesized peptides are linked to the silicon surface using a spacer that moves the peptide away from the surface so that the peptide is presented to the antibody in an unhindered orientation.

Platforms herein are also contemplated to include peptides in microtiter plates for determining T cell activity in response to frameshift peptides herein. In some embodiments, microtiter plates include but are not limited to 96 well, 384 well, 1536 well, 3 456 well, and 9600 well plates. In some embodiments, more than one peptide is present in each well of a microtiter plate, i.e., the peptides are pooled and subject peptides eliciting T cell activity are determined by deconvolution of the positive and negative wells in the T cell assay.

Optionally, it is useful to determine immunogenicity of a candidate frameshift peptide for use in a producing an antibody for a specific cancer and/or tumor type. Immunogenicity, as used herein, refers to the ability of a substance, such as a peptide, to elicit an immune response, such as an antibody response or a T cell response, when administered to a subject, for example, in a therapeutic formulation. For subjects with cancer it is the immune response to the tumor. In some embodiments, a peptide that reacts with an antibody or elicits T cell activity in a biological sample from a subject is not immunogenic when administered in a vaccine formulation. In some embodiments, a peptide that reacts with an antibody or elicits T cell activity in a biological sample from a subject is immunogenic when administered in a therapeutic formulation. Immunogenicity is determined by methods of those of skill in the art including in animal model testing and using in silico prediction of immunogenicity. In silico immunogenicity prediction tools are available for free to the public, for example at the Immune Epitope Database and Analysis Resource (www.iedb.org).

Alternatively, mice, such as mice transgenic for canine HLA genes are used to determine the immunogenicity of a candidate frameshift peptide. The candidate frameshift peptide is administered to the transgenic mouse in a therapeutic formulation. Response to the formulation is determined using antibody assays and/or T cell assays described elsewhere herein. Methods herein may include methods of determining the optimal components of an antibody to be given to a subject to treat a specific cancer in a subject such as a treatment subject. Such methods may include determining whether a candidate antibody elicits an immune response in the subject.

The variant peptides comprising the collection for screening, for example, the first population, could be from several sources. They could be peptides known to result from point mutations, frameshifts, deletions/insertions or translocation in tumor DNA. Because these types of mutations are personal and occur infrequently, it would take a large number of peptides to represent all of them. Conventional practice is to determine neo-antigens encoded at the DNA level and then confirm expression at the RNA level. However, the inventors have unexpectedly discovered that mutations occur much more frequently at the RNA level only. Since microsatellites in coding regions are predicted and limited in number, one can predict a small set of FS peptides resulting from insertion or deletions during transcription that will produce FS neo-antigens. Therefore, methods herein, in some embodiments, comprise screening frameshift variants formed from 1) insertions or deletions in microsatellites in coding regions or 2) from mis-splicing events either in or between genes that create an out-of-frame fusion. These variants have several attractive features as sources for a cancer vaccine component. First, frameshift variants generally have variant peptide sequences of over more than 8 amino acids. In contrast with point mutations that often only alters one amino acid, a FS variant is a completely foreign sequence and therefore is much more likely to be immunogenic. Work indicates that there are only a few thousand frameshifts from microsatellite insertion/deletions that are more than 8 amino acids long. Frameshifts of 8-60 amino acids long are very likely to include MHCI and MHCII epitopes. Further, because of their increased immunogenicity, FS variants are much more likely to create both T- and B-cell responses. Therefore, fewer peptides are required to be screened to determine vaccine components. Point mutation neo-epitopes are unlikely to produce both B and T-cell responses. Second, the realm of FS space is much more restricted than that of all possible point mutations. This is particularly true for indels in microsatellites in coding regions. There are 2 possible FS that can be predicted from each of the -7000 microsatellites in coding regions. These numbers become smaller as putative peptides are filtered for restrictions for minimal length (e.g., >7aa) and the probability of eliciting immune responses. This makes it feasible to have a pre-existing set of FS peptides made that can be used to screen the T-cells of a patient for reactivity.

Peptides are produced and displayed in a number of ways. For example, in some embodiments the peptide candidates are synthesized and spotted on arrays. In some embodiments, arrays have about 20, about 50, about 60, about 70, about 80, about 90 or about 100 selected FS peptides, such as those set forth in Tables 1, 2 and/or 3. In some embodiments, arrays have about 23 selected FS peptides provided in Table 3. In some embodiments, arrays have about 40 selected FS peptides. In some embodiments, arrays have about 50 selected FS peptides. In some embodiments, arrays have about 60 selected FS peptides. In some embodiments, arrays have about 70 selected FS peptides. In some embodiments, arrays have about 80 selected FS peptides. In some embodiments, arrays have about 90 selected FS peptides. In some embodiments, arrays have about 100 selected FS peptides. In some embodiments, arrays have about 200 selected FS peptides. In some embodiments, arrays have about 300 selected FS peptides. In some embodiments, arrays have about 400 selected FS peptides. In some embodiments, arrays have about 500 selected FS peptides. In some embodiments, arrays have about 600 selected FS peptides. In some embodiments, arrays have about 700 selected FS peptides. In some embodiments, arrays have about 800 selected FS peptides. In some embodiments, arrays have about 900 selected FS peptides. In some embodiments, arrays have about 1000 selected FS peptides. In some embodiments, arrays have about 10,000 selected FS peptides. In some embodiments, arrays have about 20,000 selected FS peptides. In some embodiments, in-situ synthesis could produce an array having 1,000,000 or more peptides per array, or at least 1000, 10,000 or 100,000 peptides per array.

A T-cell response, in some embodiments, is important for killing cancer cells. Since the FS peptides are generally 8 aa or longer, it is very likely that a FS peptide will have a region that would bind to the patient's MHC to initiate an immune response. MHC binding can be predicted from commonly available algorithms. Alternatively, the blood sample from the patient could be screened for T-cell activity to the peptide candidates using a T cell assay, such as a proliferation assay, a cytokine assay, a cytotoxicity assay, a degranulation assay, flow cytometry, or combination thereof.

Methods herein, in some embodiments, comprise methods of frameshift variant development for inclusion in cancer therapeutic development. Frameshift variants, as referred to herein, are alterations in an mRNA caused by errors in transcription, causing an insertion or deletion (indel) of one or two nucleotides in the mRNA or by mis-splicing of RNA resulting in a change in the amino acids of the resulting protein that are encoded after the frameshift variant. Methods of frameshift variant development herein include but are not limited to mRNA sequencing and array based hybridization. In some embodiments, frameshift peptides are developed by bioinformatics analysis of already available sequence data. FS variants peptides due to indels in MS can be directly inferred from the genome sequence data.

In some embodiments, mRNA sequencing for development of frameshift variants herein includes a method where mRNA from a tumor or cancer tissue is sequenced. In some embodiments, mRNA is purified from a tumor or cancer tissue from a patient. In some embodiments, mRNA is isolated from total mRNA from the tumor or cancer tissue. In some embodiments, mRNA is isolated using oligo-dT purification of total RNA. In some embodiments, mRNA is targeted for sequencing using an oligo-dT to prime the RNA sample. In some embodiments, the mRNA is amplified before sequencing. In some embodiments, the mRNA is amplified by PCR before sequencing. In some embodiments the mRNA is amplified by RT-PCR before sequencing. In some embodiments, mRNA sequencing comprises targeted sequencing of an mRNA having a microsatellite in the transcript. In some embodiments, mRNA is sequenced using at least one technique selected from Sanger sequencing, pyro-sequencing, ion semiconductor sequencing, polony sequencing, sequencing by ligation, nanoball sequencing, and single molecule sequencing.

Variants identified from mRNA sequencing are classified by type of variant. Variants may arise from mutations in DNA or alterations in the RNA during transcription or splicing herein, which include but are not limited to point mutations, silent mutations, insertions, deletions, cis-splicing errors, and trans-splicing errors. Of these, only insertions, deletions, cis-splicing errors, and trans-splicing errors are expected to lead to a frameshift in a protein produced from the mutant mRNA. Confirmed frameshift variants are those that when translated produce a protein with a different amino acid sequence at more than one residue at residues C-terminal to the alteration. Frameshifted polypeptide sequences resulting from frameshift variants are assembled for further analysis.

In some cases, frameshift mutations are predicted based on microsatellite location in the genome. As transcripts having a microsatellite are more prone to transcription errors, frameshift polypeptides can be predicted to be resulting from an insertion or a deletion of one or two basepairs. Alternatively, frameshift polypeptides can be predicted by bioinformatics prediction of cis and/or trans splicing errors. A selection of all possible frameshift peptides can be assembled for further analysis.

Frameshifted polypeptide sequences, determined by mRNA sequencing or prediction, are further analyzed to determine immunoreactivity. In some embodiments, immunoreactivity is measured by MHC or HLA binding. In some embodiments, immunoreactivity is measured by antibody binding. In some embodiments, immunoreactivity is measured by T cell activity. In some embodiments, immunoreactivity is measured by antibody binding and T cell activity.

Binding to MHC is required for T cell activity and can be determined by binding assays. Alternatively, in silico methods of MHC binding are used to predict binding of a peptide to a MHC subtype. Data of peptides binding to MHC subtype molecules are used to develop binding prediction algorithms. These algorithms calculate scoring matrices that quantify the contribution of each residue in a fixed-length peptide to binding to an MHC molecule. Algorithms predict binding of a peptide to class I MHC or class II MHC. Algorithms to predict class I MHC binding include but are not limited to Artificial neural network (ANN), Stabilized matrix method (SMM), SMM with a Peptide:MHC Binding Energy Covariance matrix (SMMPMBEC), Scoring Matrices derived from Combinatorial Peptide Libraries (Comblib_Sidney2008), Consensus, NetMHCpan, NetMHCcons and PickPocket. Algorithms to predict class II MHC binding, include but are not limited to Consensus method, Combinatorial library, NN-align (netMHCII-2.2), SMM-align (netMHCII-1.1), Sturniolo, and NetMHCIIpan. The entire population of frameshift polypeptides is then scanned using one or more of the above algorithms for peptides binding to an MHC subtype molecule with a predicted affinity of IC50<500 nM.

iii. Antibodies

The term “antibodies” is used herein in a broad sense and includes both polyclonal and monoclonal antibodies. In addition to intact immunoglobulin molecules, also included in the term “antibodies” are fragments or polymers of those immunoglobulin molecules, and human or humanized versions of immunoglobulin molecules or fragments thereof, as long as they are chosen for their ability to interact with their specific target and bring about the desired outcome, such as inhibition or reduction of tumor growth. The antibodies can be tested for their desired activity using the in vitro assays described herein, or by analogous methods, after which their in vivo therapeutic and/or prophylactic activities are tested according to known clinical testing methods.

As used herein, the term “antibody” encompasses, but is not limited to, whole immunoglobulin (i.e., an intact antibody) of any class. Native antibodies are usually heterotetrameric glycoproteins, composed of two identical light (L) chains and two identical heavy (H) chains. Typically, each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (V(H)) followed by a number of constant domains. Each light chain has a variable domain at one end (V(L)) and a constant domain at its other end; the constant domain of the light chain is aligned with the first constant domain of the heavy chain, and the light chain variable domain is aligned with the variable domain of the heavy chain. Particular amino acid residues are believed to form an interface between the light and heavy chain variable domains. The light chains of antibodies from any vertebrate species can be assigned to one of two clearly distinct types, called kappa (κ) and lambda (1), based on the amino acid sequences of their constant domains. Depending on the amino acid sequence of the constant domain of their heavy chains, immunoglobulins can be assigned to different classes. There are five major classes of human immunoglobulins: IgA, IgD, IgE, IgG and IgM, and several of these may be further divided into subclasses (isotypes), e.g., IgG-I, IgG-2, IgG-3, and IgG-4; IgA-I and IgA-2. One skilled in the art would recognize the comparable classes for mouse. The heavy chain constant domains that correspond to the different classes of immunoglobulins are called alpha, delta, epsilon, gamma, and mu, respectively.

The term “variable” is used herein to describe certain portions of the variable domains that differ in sequence among antibodies and are used in the binding and specificity of each particular antibody for its particular antigen. However, the variability is not usually evenly distributed through the variable domains of antibodies. It is typically concentrated in three segments called complementarity determining regions (CDRs) or hypervariable regions both in the light chain and the heavy chain variable domains. The more highly conserved portions of the variable domains are called the framework (FR). The variable domains of native heavy and light chains each comprise four FR regions, largely adopting a b-sheet configuration, connected by three CDRs, which form loops connecting, and in some cases forming part of, the b-sheet structure. The CDRs in each chain are held together in close proximity by the FR regions and, with the CDRs from the other chain, contribute to the formation of the antigen binding site of antibodies (see Kabat E. A. et al., “Sequences of Proteins of Immunological Interest,” National Institutes of Health, Bethesda, Md.). The constant domains are not involved directly in binding an antibody to an antigen, but exhibit various effector functions, such as participation of the antibody in antibody-dependent cellular toxicity.

iv. Biological Samples

The methods and arrays disclosed herein utilize small quantities of biological samples from a subject. In some embodiments, the biological samples can be used in a disclosed method without further processing and in small quantities. In some embodiments, the biological samples comprise, blood, serum, saliva, sweat, cells, tissues, or any bodily fluid. In some embodiments, about 0.5 nl, about 1 nl, about 2 nl, about 3 nl, about 4 nl, about 5 nl, about 6 nl, about 7 nl, about 8 nl, about 9 nl, about 10 nl, about 11 nl, about 12 nl, about 13 nl, about 14 nl, about 15 nl, about 16 nl, about 17 nl, about 18 nl, about 19 nl, about 20 nl, about 21 nl, about 22 nl, about 23 nl, about 24 nl, about 25 nl, about 26 nl, about 27 nl, about 28 nl, about 29 nl, about 30 nl, about 31 nl, about 32 nl, about 33 nl, about 34 nl, about 35 nl, about 36 nl, about 37 nl, about 38 nl, about 39 nl, about 40 nl, about 41 nl, about 42 nl, about 43 nl, about 44 nl, about 45 nl, about 46 nl, about 47 nl, about 48 nl, about 49 nl, or about 50 nl, about 51 nl, about 52 nl, about 53 nl, about 54 nl, about 55 nl, about 56 nl, about 57 nl, about 58 nl, about 59 nl, about 60 nl, about 61 nl, about 62 nl, about 63 nl, about 64 nl, about 65 nl, about 66 nl, about 67 nl, about 68 nl, about 69 nl, about 70 nl, about 71 nl, about 72 nl, about 73 nl, about 74 nl, about 75 nl, about 76 nl, about 77 nl, about 78 nl, about 79 nl, about 80 nl, about 81 nl, about 82 nl, about 83 nl, about 84 nl, about 85 nl, about 86 nl, about 87 nl, about 88 nl, about 89 nl, about 90 nl, about 91 nl, about 92 nl, about 93 nl, about 94 nl, about 95 nl, about 96 nl, about 97 nl, about 98 nl, about 99 nl, about 0.1, about 0.2 μl, about 0.3 μl, about 0.4 μl, about 0.5 μl, about 0.6 μl. about 0.7 μl, about 0.8 μl, about 0.9 μl, about 1 μl, about 2 μl, about 3 μl, about 4 μl, about 5 μl, about 6 μl, about 7 μl, about 8 μl, about 9 μl, about 10 μl, about 11 μl, about 12 μl, about 13 μl, about 14 μl, about 15 μl, about 16 μl, about 17 μl, about 18 μl, about 19 μl, about 20 μl, about 21 μl, about 22 μl, about 23 μl, about 24 μl, about 25 μl, about 26 μl, about 27 μl, about 28 μl, about 29 μl, about 30 μl, about 31 μl, about 32 μl, about 33 μl, about 34 μl, about 35 μl, about 36 μl, about 37 μl, about 38 μl, about 39 μl, about 40 μl, about 41 μl, about 42 μl, about 43 μl, about 44 μl, about 45 μl, about 46 μl, about 47 μl, about 48 μl, about 49 μl, or about 50 of biological samples are required for analysis by an array.

A biological sample from a subject can be for example, collected from a subject and directly contacted with an array. In some embodiments, the biological sample does not require a preparation or processing step prior to being contacted with an array as described herein. In some embodiments, a dry blood sample from a subject is reconstituted in a dilution step prior to being contacted with an array. A dilution can provide an optimum concentration of an antibody from a biological sample of a subject for testing according to the methods disclosed herein.

In some embodiments, the disclosed methods require no more than about 0.5 nl to about 50 nl, no more than about 1 nl to about 100 nl, no more than about 1 nl to about 150 nl, no more than about 1 nl to about 200 nl, no more than about 1 nl to about 250 nl, no more than about 1 nl to about 300 nl, no more than about 1 nl to about 350 nl, no more than about 1 nl to about 400 nl, no more than about 1 to about 450 nl, no more than about 5 nl to about 500 nl, no more than about 5 nl to about 550 nl, no more than about 5 nl to about 600 nl, no more than about 5 nl to about 650 nl, no more than about 5 nl to about 700 nl, no more than about 5 nl to about 750 nl, no more than about 5 nl to about 800 nl, no more than about 5 nl to about 850 nl, no more than about 5 nl to about 900 nl, no more than about 5 nl to about 950 nl, no more than about 5 nl to about 1 μl, no more than about 0.5 μl to about 1 μl, no more than about 0.5 μl to about 5 μl, no more than about 1 μl to about 10 μl, no more than about 1 μl to about 20 μl, no more than about 1 μl to about 30 μl, no more than about 1 μl to about 40 μl, or no more than about 1 μl to about 50 μl.

In some embodiments, the methods utilize at least 0.5 nl to about 50 nl, at least about 1 nl to about 100 nl, at least about 1 nl to about 150 nl, at least about 1 nl to about 200 nl, at least about 1 nl to about 250 nl, at least about 1 nl to about 300 nl, at least about 1 nl to about 350 nl, at least about 1 nl to about 400 nl, at least about 1 to about 450 nl, at least about 5 nl to about 500 nl, at least about 5 nl to about 550 nl, at least about 5 nl to about 600 nl, at least about 5 nl to about 650 nl, at least about 5 nl to about 700 nl, at least about 5 nl to about 750 nl, at least about 5 nl to about 800 nl, at least about 5 nl to about 850 nl, at least about 5 nl to about 900 nl, at least about 5 nl to about 950 nl, at least about 5 nl to about 1 μl, at least about 0.5 μl to about 1 μl, at least about 0.5 μl to about 5 μl, at least about 1 μl to about 10 μl, at least about 1 IA to about 20 μl, at least about 1 μl to about 30 μl, at least about 1 μl to about 40 μl, at least about 1 μl to about 50 μl, or at least 50 μl.

In some embodiments, biological samples from a subject are too concentrated and require a dilution prior to being contacted with a disclosed array. A plurality of dilutions can be applied to a biological sample prior to contacting the sample with an array. A dilution can be a serial dilution, which can result in a geometric progression of the concentration in a logarithmic fashion. For example, a ten-fold serial dilution can be 1 M, 0.01 M, 0.001 M, and a geometric progression thereof. A dilution can be, for example, a one-fold dilution, a two-fold dilution, a three-fold dilution, a four-fold dilution, a five-fold dilution, a six-fold dilution, a seven-fold dilution, an eight-fold dilution, a nine-fold dilution, a ten-fold dilution, a sixteen-fold dilution, a twenty-five-fold dilution, a thirty-two-fold dilution, a sixty-four-fold dilution, and/or a one-hundred-and-twenty-five-fold dilution.

A biological sample can be derived from a plurality of sources within a subject's body and a biological sample can be collected from a subject in a plurality of different circumstances. A biological sample can be collected, for example, during a routine medical consultation, such as a blood draw during an annual physical examination. A biological sample can be collected during the course of a non-routine consultation, for example, a biological sample can be collected during the course of determining treatment for a given tumor or cancer. A subject can also collect a biological sample from oneself, and a subject can provide a biological sample to be analyzed by the methods and systems as provided herein in a direct-to-consumer fashion. In some embodiments, a biological sample can be mailed to a provider of the methods and arrays of embodiments provided herein. In some embodiments, a dry biological sample, such as a dry blood sample from a subject on a filter paper, is mailed to a provider of the methods and arrays of the embodiments provided herein.

v. Cancer and Tumor Type

In some embodiments, the cancer and/or tumor type comprises a MSI-H cancer and/or tumor. In some embodiments, the cancer and/or tumor type is a CPI responsive cancer and/or tumor type. In some embodiments, the cancer and/or tumor type is selected from bladder cancer, lung cancer, kidney cancer, melanoma, head cancer, neck cancer, Hodgkin's lymphoma and solid tumors. In some embodiments, the cancer and/or tumor is one of the following: Acanthoma, Acinic cell carcinoma, Acoustic neuroma, Acral lentiginous melanoma, Acrospiroma, Acute eosinophilic leukemia, Acute lymphoblastic leukemia, Acute megakaryoblastic leukemia, Acute monocytic leukemia, Acute myeloblastic leukemia with maturation, Acute myeloid dendritic cell leukemia, Acute myeloid leukemia, Acute promyelocytic leukemia, Adamantinoma, Adenocarcinoma, Adenoid cystic carcinoma, Adenoma, Adenomatoid odontogenic tumor, Adrenocortical carcinoma, Adult T-cell leukemia, Aggressive NK-cell leukemia, AIDS-Related Cancers, AIDS-related lymphoma, Alveolar soft part sarcoma, Ameloblastic fibroma, Anal cancer, Anaplastic large cell lymphoma, Anaplastic thyroid cancer, Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone Cancer, Bone tumor, Brain Stem Glioma, Brain Tumor, Breast Cancer, Brenner tumor, Bronchial Tumor, Bronchioloalveolar carcinoma, Brown tumor, Burkitt's lymphoma, Cancer of Unknown Primary Site, Carcinoid Tumor, Carcinoma, Carcinoma in situ, Carcinoma of the penis, Carcinoma of Unknown Primary Site, Carcinosarcoma, Castleman's Disease, Central Nervous System Embryonal Tumor, Cerebellar Astrocytoma, Cerebral Astrocytoma, Cervical Cancer, Cholangiocarcinoma, Chondroma, Chondrosarcoma, Chordoma, Choriocarcinoma, Choroid plexus papilloma, Chronic Lymphocytic Leukemia, Chronic monocytic leukemia, Chronic myelogenous leukemia, Chronic Myeloproliferative Disorder, Chronic neutrophilic leukemia, Clear-cell tumor, Colon Cancer, Colorectal cancer, Craniopharyngioma, Cutaneous T-cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial Uterine Cancer, Endometrioid tumor, Enteropathy-associated T-cell lymphoma, Ependymoblastoma, Ependymoma, Epithelioid sarcoma, Erythroleukemia, Esophageal cancer, Esthesioneuroblastoma, Ewing Family of Tumor, Ewing Family Sarcoma, Ewing's sarcoma, Extracranial Germ Cell Tumor, Extragonadal Germ Cell Tumor, Extrahepatic Bile Duct Cancer, Extramammary Paget's disease, Fallopian tube cancer, Fetus in fetu, Fibroma, Fibrosarcoma, Follicular lymphoma, Follicular thyroid cancer, Gallbladder Cancer, Gallbladder cancer, Ganglioglioma, Ganglioneuroma, Gastric Cancer, Gastric lymphoma, Gastrointestinal cancer, Gastrointestinal Carcinoid Tumor, Gastrointestinal Stromal Tumor, Gastrointestinal stromal tumor, Germ cell tumor, Germinoma, Gestational choriocarcinoma, Gestational Trophoblastic Tumor, Giant cell tumor of bone, Glioblastoma multiforme, Glioma, Gliomatosis cerebri, Glomus tumor, Glucagonoma, Gonadoblastoma, Granulosa cell tumor, Hairy Cell Leukemia, Hairy cell leukemia, Head and Neck Cancer, Head and neck cancer, Heart cancer, Hemangioblastoma, Hemangiopericytoma, Hemangiosarcoma, Hematological malignancy, Hepatocellular carcinoma, Hepatosplenic T-cell lymphoma, Hereditary breast-ovarian cancer syndrome, Hodgkin Lymphoma, Hodgkin's lymphoma, Hypopharyngeal Cancer, Hypothalamic Glioma, Inflammatory breast cancer, Intraocular Melanoma, Islet cell carcinoma, Islet Cell Tumor, Juvenile myelomonocytic leukemia, Kaposi Sarcoma, Kaposi's sarcoma, Kidney Cancer, Klatskin tumor, Krukenberg tumor, Laryngeal Cancer, Laryngeal cancer, Lentigo maligna melanoma, Leukemia, Lip and Oral Cavity Cancer, Liposarcoma, Lung cancer, Luteoma, Lymphangioma, Lymphangiosarcoma, Lymphoepithelioma, Lymphoid leukemia, Lymphoma, Macroglobulinemia, Malignant Fibrous Histiocytoma, Malignant fibrous histiocytoma, Malignant Fibrous Histiocytoma of Bone, Malignant Glioma, Malignant Mesothelioma, Malignant peripheral nerve sheath tumor, Malignant rhabdoid tumor, Malignant triton tumor, MALT lymphoma, Mantle cell lymphoma, Mast cell leukemia, Mediastinal germ cell tumor, Mediastinal tumor, Medullary thyroid cancer, Medulloblastoma, Medulloepithelioma, Melanoma, Meningioma, Merkel Cell Carcinoma, Mesothelioma, Metastatic Squamous Neck Cancer with Occult Primary, Metastatic urothelial carcinoma, Mixed Mullerian tumor, Monocytic leukemia, Mouth Cancer, Mucinous tumor, Multiple Endocrine Neoplasia Syndrome, Multiple myeloma, Mycosis Fungoides, Myelodysplastic Disease, Myelodysplastic Syndromes, Myeloid leukemia, Myeloid sarcoma, Myeloproliferative Disease, Myxoma, Nasal Cavity Cancer, Nasopharyngeal Cancer, Nasopharyngeal carcinoma, Neoplasm, Neurinoma, Neuroblastoma, Neurofibroma, Neuroma, Nodular melanoma, Non-Hodgkin lymphoma, Nonmelanoma Skin Cancer, Non-Small Cell Lung Cancer, Ocular oncology, Oligoastrocytoma, Oligodendroglioma, Oncocytoma, Optic nerve sheath meningioma, Oral cancer, Oropharyngeal Cancer, Osteosarcoma, Ovarian cancer, Ovarian Epithelial Cancer, Ovarian Germ Cell Tumor, Ovarian Low Malignant Potential Tumor, Paget's disease of the breast, Pancoast tumor, Pancreatic cancer, Papillary thyroid cancer, Papillomatosis, Paraganglioma, Paranasal Sinus Cancer, Parathyroid Cancer, Penile Cancer, Perivascular epithelioid cell tumor, Pharyngeal Cancer, Pheochromocytoma, Pineal Parenchymal Tumor of Intermediate Differentiation, Pineoblastoma, Pituicytoma, Pituitary adenoma, Pituitary tumor, Plasma Cell Neoplasm, Pleuropulmonary blastoma, Polyembryoma, Precursor T-lymphoblastic lymphoma, Primary central nervous system lymphoma, Primary effusion lymphoma, Primary Hepatocellular Cancer, Primary Liver Cancer, Primary peritoneal cancer, Primitive neuroectodermal tumor, Prostate cancer, Pseudomyxoma peritonei, Rectal Cancer, Renal cell carcinoma, Respiratory Tract Carcinoma Involving the NUT Gene on Chromosome 15, Retinoblastoma, Rhabdomyoma, Rhabdomyosarcoma, Richter's transformation, Sacrococcygeal teratoma, Salivary Gland Cancer, Sarcoma, Schwannomatosis, Sebaceous gland carcinoma, Secondary neoplasm, Seminoma, Serous tumor, Sertoli-Leydig cell tumor, Sex cord-stromal tumor, Sezary Syndrome, Signet ring cell carcinoma, Skin Cancer, Small blue round cell tumor, Small cell carcinoma, Small Cell Lung Cancer, Small cell lymphoma, Small intestine cancer, Soft tissue sarcoma, Somatostatinoma, Soot wart, Spinal Cord Tumor, Spinal tumor, Splenic marginal zone lymphoma, Squamous cell carcinoma, Stomach cancer, Superficial spreading melanoma, Supratentorial Primitive Neuroectodermal Tumor, Surface epithelial-stromal tumor, Synovial sarcoma, T-cell acute lymphoblastic leukemia, T-cell large granular lymphocyte leukemia, T-cell leukemia, T-cell lymphoma, T-cell prolymphocytic leukemia, Teratoma, Terminal lymphatic cancer, Testicular cancer, Thecoma, Throat Cancer, Thymic Carcinoma, Thymoma, Thyroid cancer, Transitional Cell Cancer of Renal Pelvis and Ureter, Transitional cell carcinoma, Urachal cancer, Urethral cancer, Urogenital neoplasm, Uterine sarcoma, Uveal melanoma, Vaginal Cancer, Verner Morrison syndrome, Verrucous carcinoma, Visual Pathway Glioma, Vulvar Cancer, Waldenstrom's macroglobulinemia, Warthin's tumor, or Wilms' tumor. Thus, using the methods disclosed herein the MSI and/or immune response to any of the listed cancer and/or tumor types as well as any that are not listed can be determined and monitored.

vi. Administering a Treatment

In some embodiments, the method includes administering a treatment to the subject, prior to or following determining MSI status. For example, after determining MSI status as high, in current standards, one or more CPI inhibitors is administered at an effective concentration thereby treating the cancer and/or tumor in the subject in need thereof.

Methods can include any known treatment used to control tumor growth, size, metastasis or other desired tumor activity or characteristic. In some examples, treatment can include radiation, surgical removal and/or administration of a composition such as a composition including one or more CPIs. In some embodiments, the cancer and/or tumor type comprises a MSI-H cancer and/or tumor. In some embodiments, the cancer and/or tumor type is a CPI responsive cancer and/or tumor type. In some embodiments, the cancer and/or tumor type is selected from bladder cancer, lung cancer, kidney cancer, melanoma, head cacner, neck cancer, Hodgkin's lymphoma and solid tumors. In some embodiments, the methods are used to treat any cancer and/or tumor type that is MSI-H.

Effective dosages and schedules for administering the compositions may be determined empirically, and making such determinations is within the skill in the art. The dosage ranges for the administration of the compositions are those large enough to produce the desired effect in which the symptoms/disorder are/is affected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 mg/kg to up to 100 mg/kg of body weight or more per day, depending on the factors mentioned above.

Following treatment, including administration of an antibody, for treating, inhibiting, or preventing a cancer/tumor, the efficacy can be assessed by obtaining a sample and applying the sample to a FSP array and detecting the presence of antibodies and comparing the antibody concentration to that observed prior to treatment.

### IV. Vaccines

Disclosed herein are vaccines composed of frameshift peptide (FSP) neo-antigens that are commonly produced in MSI-H cancer/tumor types or nucleic acids encoding the same. The inventors have identified 100 FSP neo-antigens that are commonly produced in subjects with MSI-H cancer or tumor types and these FSPs are immunogenic as measured by antibody reactivity.

In embodiments, a MSI-H vaccine includes one or more peptides having the sequence according to one or more peptides provided in Tables 1, 2 and/or 3 and/or a nucleic acid capable of expressing the one or more peptides and a pharmaceutically acceptable carrier. The vaccine maybe separated into its constituent components, such as one more nucleic acid components, and/or peptide components, for example as part of a prime/boost vaccine strategy.

In certain embodiments, the vaccine includes one or more vectors expressing the peptide scoring to SEQ ID Nos. provided in Table 3. In some examples, the amino acid sequences encoded by the nucleic acids are separated by a peptide linker. Peptide linkers are known in the art and include for example poly Gly-Ser.

In embodiments, the vaccine includes a peptide component, for example, as part of a prime-boost protocol, such as where the nucleic acid component is given first and followed at some time later the peptide component.

The vaccine compositions also can be formulated to contain an adjuvant in order to enhance the immunological response. Suitable adjuvants include, but are not limited to, mineral gels such as aluminum hydroxide, surface active substances such as lysolecithin, pluronic polyols, polyanions, other peptides, oil emulsions, and potentially useful human adjuvants such as Bacillus Calmette Guerin (BCG) and Corynebacterium parvum. Adjuvants for inclusion in the inventive composition desirably are safe, well tolerated, such as QS-21, Detox-PC, MPL-SE, MoGM-CSF, TiterMax-G, CRL-1005, GERBU, TERamide, PSC97B, Adjumer, PG-026, GSK-1, GcMAF, B-alethine, MPC-026, Adjuvax, CpG ODN, Betafectin, Alum, and MF59 (as described in, e.g., Kim et al., Vaccine, 18: 597 (2000)). Other adjuvants that can be administered to a mammal include lectins, growth factors, cytokines, and lymphokines (e.g., alpha-interferon, gamma-interferon, platelet derived growth factor (PDGF), gCSF, gMCSF, TNF, epidermal growth factor (EGF), IL-1, IL-2, IL-4, IL-6, IL-8, IL-10, and IL-12). ABM2, AS01B, AS02, AS2A, Adjumer, Adjuvax, Algammulin, Alum, Alumnuwm phosphate, Aluminum potassiurn sulfate, Bordetella pertussis, Calcitriol, Chitosan, Cholera toxin, CpG Dibutyl phthalate, Dimethyldioctadecylammonium bromide (DDA), Freund's adjuvait, Freund's complete, Freund's incomplete (IF A), GM-CSF, GMDP, Gamma Inulin, Glycerol, HBSS (Hank's Balanced Salt Solution), Imiquirnod, Interferon-Gamma, ISCOM, Lipid Core Peptide (LCP), Lipofectin, Lipopolysaccharide (LPS), Liposomes, MF59, MLP+TDM, Monophosphoryl lipid A, Montanide IMS-1313, Montanide ISA 206, Montanide ISA 720, Montanide ISA-51, Montanide ISA-50, nor-MDP, Oil-in-water emulsion, P1005 (non-ionic copolymer), Pan3Cys (lipoprotein), Pertussis toxin, Poloxamer, QS21, RaLPS, Ribi, Saponin, Seppic ISA 720, Soybean Oil, Squalene, Syntex Adjuvant Forinulation (SAF), Synthetic polynucleotides (poly IC/poly AU), TiterMax Tomatine, Vaxfectin, XtendIII, and Zymosan. Checkpoint inhibitors can also be used. Some such checkpoint inhibitors are selected from the group consisting of a PD-1 inhibitor, a PD-L1 inhibitor, and a CTLA-4 inhibitor.

i. Polynucleotides Encoding MSI-H FSP Neoantigens

Polynucleotides encoding the antigenic peptide disclosed herein are provided. These polynucleotides include DNA, cDNA and RNA sequences which encode the antigen.

Methods for the manipulation and insertion of the nucleic acids of this disclosure into vectors are well known in the art (see for example, Sambrook et al., Molecular Cloning, a Laboratory Manual, 2d edition, Cold Spring Harbor Press, Cold Spring Harbor, N.Y., 1989, and Ausubel et al., Current Protocols in Molecular Biology, Greene Publishing Associates and John Wiley & Sons, New York, N.Y., 1994).

A nucleic acid encoding an antigenic peptide can be cloned or amplified by in vitro methods, such as the polymerase chain reaction (PCR), the ligase chain reaction (LCR), the transcription-based amplification system (TAS), the self-sustained sequence replication system (3SR) and the QP replicase amplification system (QB). For example, a polynucleotide encoding the protein can be isolated by polymerase chain reaction of cDNA using primers based on the DNA sequence of the molecule. A wide variety of cloning and in vitro amplification methodologies are well known to persons skilled in the art. PCR methods are described in, for example, U.S. Pat. No. 4,683,195; Mullis et al., Cold Spring Harbor Symp. Quant. Biol. 51:263, 1987; and Erlich, ed., PCR Technology, (Stockton Press, N Y, 1989). Polynucleotides also can be isolated by screening genomic or cDNA libraries with probes selected from the sequences of the desired polynucleotide under stringent hybridization conditions.

The polynucleotides encoding an antigen include a recombinant DNA which is incorporated into a vector into an autonomously replicating plasmid or virus or into the genomic DNA of a prokaryote or eukaryote, or which exists as a separate molecule (such as a cDNA) independent of other sequences. The nucleotides of embodiments provided herein can be ribonucleotides, deoxyribonucleotides, or modified forms of either nucleotide. The term includes single and double forms of DNA.

DNA sequences encoding the antigen can be expressed in vitro by DNA transfer into a suitable host cell. The cell may be prokaryotic or eukaryotic. The term also includes any progeny of the subject host cell. It is understood that all progeny may not be identical to the parental cell since there may be mutations that occur during replication. Methods of stable transfer, meaning that the foreign DNA is continuously maintained in the host, are known in the art.

Polynucleotide sequences encoding antigens can be operatively linked to expression control sequences. An expression control sequence operatively linked to a coding sequence is ligated such that expression of the coding sequence is achieved under conditions compatible with the expression control sequences. The expression control sequences include, but are not limited to, appropriate promoters, enhancers, transcription terminators, a start codon (i.e., ATG) in front of a protein-encoding gene, splicing signal for introns, maintenance of the correct reading frame of that gene to permit proper translation of mRNA, and stop codons.

Hosts can include microbial, yeast, insect and mammalian organisms.

Methods of expressing DNA sequences having eukaryotic or viral sequences in prokaryotes are well known in the art. Non-limiting examples of suitable host cells include bacteria, archea, insect, fungi (for example, yeast), plant, and animal cells (for example, mammalian cells, such as canine cells). Exemplary cells of use include Escherichia coli, Bacillus subtilis, Saccharomyces cerevisiae, Salmonella typhimurium, SF9 cells, C129 cells, 293 cells, Neurospora, and immortalized mammalian myeloid and lymphoid cell lines. Techniques for the propagation of mammalian cells in culture are well-known (see, Jakoby and Pastan (eds), 1979, Cell Culture. Methods in Enzymology, volume 58, Academic Press, Inc., Harcourt Brace Jovanovich, N.Y.). Examples of commonly used mammalian host cell lines are VERO and HeLa cells, CHO cells, and WI38, BHK, and COS cell lines, although cell lines may be used, such as cells designed to provide higher expression desirable glycosylation patterns, or other features.

Transformation of a host cell with recombinant DNA can be carried out by conventional techniques as are well known to those skilled in the art. Where the host is prokaryotic, such as, but not limited to, E. coli, competent cells which are capable of DNA uptake can be prepared from cells harvested after exponential growth phase and subsequently treated by the CaCl₂) method using procedures well known in the art. Alternatively, MgCl₂ or RbCl can be used. Transformation can also be performed after forming a protoplast of the host cell if desired, or by electroporation.

When the host is a eukaryote, such methods of transfection of DNA as calcium phosphate coprecipitates, conventional mechanical procedures such as microinjection, electroporation, insertion of a plasmid encased in liposomes, or viral vectors can be used. Eukaryotic cells can also be co-transformed with polynucleotide sequences encoding an antigen, and a second foreign DNA molecule encoding a selectable phenotype, such as the herpes simplex thymidine kinase gene. Another method is to use a eukaryotic viral vector, such as simian virus 40 (SV40) or bovine papilloma virus, to transiently infect or transform eukaryotic cells and express the protein (see for example, Eukaryotic Viral Vectors, Cold Spring Harbor Laboratory, Gluzman ed., 1982).

In some embodiments, a nucleic acid molecule that encodes an antigenic peptide is a nucleic acid provided herein as one that encodes any one of amino acid sequences provided in any one of Tables 1, 2 and/or 3. In some embodiments, a nucleic acid molecule that encodes an antigen comprises a nucleic acid sequence at least about 95% identical, such as about 95%, about 96%, about 97%, about 98%, about 99% or even 100% identical to the nucleic acid sequence encoding any one of peptides provided in Tables 1, 2 and/or 3. In some embodiments, a nucleic acid molecule that encodes an antigen consists of a nucleic acid sequence encoding any one of the peptides provided in Tables 1, 2 and/or 3 or at least 6, at least 7, at least 8, at least 9 or more consecutive amino acids, such as between 6-30, 8-30, 8-20, 10 to 25 amino acids of any one of the sequences provided in Tables 1, 2 and/or 3.

ii. Vectors

The nucleic acid molecules encoding the antigenic peptides disclosed herein can be included in a vector, for example for expression of the antigen in a host cell, or for immunization of a subject as disclosed herein. In some embodiments, the vectors are administered to a subject as part of a prime-boost vaccination. In several embodiments, the vectors are included in a vaccine, such as a booster vaccine for use in a prime-boost vaccination.

iii. Therapeutic Methods and Pharmaceutical Compositions

Disclosed are methods of treating, inhibiting, and/or preventing cancer in a subject, for example by inducing an immune response, such as a protective immune response in a subject. In some embodiments, the disclosed methods include administering to the subject a vaccine including one or more of the immunogenic peptides disclosed herein, for example as isolated peptides and/or nucleic acids encoding the peptides. In some embodiments, the disclosed methods include administering to the subject a vaccine including a nucleic acid encoding one or more of the immunogenic peptides disclosed herein. In some embodiments, the disclosed methods include administering to the subject include administering to the subject a vaccine including one or more of the immunogenic peptides disclosed herein and a vaccine including a nucleic acid encoding one or more of the immunogenic peptides disclosed herein. In some examples the vaccination including administering a priming vaccine and then, after a period of time has past, administering to the subject a boosting vaccine, for example a peptide vaccine followed by an nucleic acid vaccine. The immune response is “primed” upon administration of the priming vaccine, and is “boosted” upon administration of the boosting vaccine.

The booster vaccine is administered to the subject after the primer vaccine. Administration of the priming vaccine and the boosting vaccine can be separated by any suitable timeframe. For example, the booster vaccine can be administered at least 1 week (e.g., 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 24 weeks, 28 weeks, 35 weeks, 40 weeks, 50 weeks, or at least 52 weeks, or a range defined by any two of the foregoing values) following administration of the first immunogenic compound. In some embodiments, the booster vaccine can be administered at about 1 week, 2 weeks 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 24 weeks, 28 weeks, 35 weeks, 40 weeks, 50 weeks, or about 52 weeks, or a range defined by any two of the foregoing values, following administration of the first immunogenic compound. More than one dose of priming vaccine and/or boosting vaccine can be provided in any suitable timeframe. The dose of the priming vaccine and boosting vaccine administered to the mammal depends on a number of factors, including the extent of any side-effects, the particular route of administration, and the like.

The methods can include selecting a subject in need of treatment, such as a subject at risk of or afflicted with a MSI-associated cancer, such as a MSI-H cancer.

In embodiments a vaccine, such as a single vaccine or a prime and boost vaccine are typically administered as a pharmaceutically acceptable (e.g., physiologically acceptable) composition, which comprises a carrier, preferably a pharmaceutically carrier (e.g., physiologically acceptable). The vaccines can be administered alone, or in combination with at least one additional immunogenic agent or composition. It will be understood by those of skill in the art that the ability to produce an immune response after exposure to an antigen is a function of complex cellular and humoral processes, and that different subjects have varying capacity to respond to an immunological stimulus. Accordingly, the compositions disclosed herein are capable of eliciting an immune response in an immunocompetent subject, that is a subject that is physiologically capable of responding to an immunological stimulus by the production of a substantially normal immune response, e.g., including the production of antibodies that specifically interact with the immunological stimulus, and/or the production of functional T-cells (CD4+ and/or CD8+ T-cells) that bear receptors that specifically interact with the immunological stimulus.

Suitable formulations for the compositions include aqueous and non-aqueous solutions, isotonic sterile solutions, which can contain anti-oxidants, buffers, and bacteriostats, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid carrier, for example, water, immediately prior to use. Extemporaneous solutions and suspensions can be prepared from sterile powders, granules, and tablets. Preferably, the carrier is a buffered saline solution. The compositions can be formulated to protect the nucleic acid sequence or vector from damage prior to administration. For example, the pharmaceutical composition can be formulated to reduce loss of the nucleic acid or construct on devices used to prepare, store, or administer the composition, such as glassware, syringes, or needles. The composition can be formulated to decrease the light sensitivity and/or temperature sensitivity of the nucleic acid sequence or construct. To this end, the composition preferably comprises a pharmaceutically acceptable liquid carrier, such as, for example, those described above, and a stabilizing agent selected from the group consisting of Polysorbate 80, L-arginine, polyvinylpyrrolidone, trehalose, and combinations thereof. Use of such a composition will extend the shelf life of the nucleic acid sequence or construct, facilitate administration, and increase the efficiency of the inventive method.

A composition also can be formulated to enhance transduction efficiency of the nucleic acid molecule or construct. In addition, one of ordinary skill in the art will appreciate that the composition can comprise other therapeutic or biologically-active agents. For example, factors that control inflammation, such as ibuprofen or steroids, can be part of the composition to reduce swelling and inflammation associated with in vivo administration of the composition. Antibiotics, e.g., microbicides and fungicides, can be present to treat existing infection and/or reduce the risk of future infection, such as infection associated with gene transfer procedures.

The composition also can be formulated to contain an adjuvant in order to enhance the immunological response. Suitable adjuvants include, but are not limited to, lysolecithin, pluronic polyols, polyanions, other peptides, oil emulsions, and potentially useful human adjuvants such as Bacillus Calmette Guerin (BCG) and Corynebacterium parvum. Adjuvants for inclusion in the inventive composition desirably are safe, well tolerated, such as QS-21, Detox-PC, MPL-SE, MoGM-CSF, TiterMax-G, CRL-1005, GERBU, TERamide, PSC97B, Adjumer, PG-026, GSK-1, GcMAF, B-alethine, MPC-026, Adjuvax, CpG ODN, Betafectin, Alum, and MF59 (as described in, e.g., Kim et al., Vaccine, 18: 597 (2000)). Other adjuvants that can be administered to a mammal include lectins, growth factors, cytokines, and lymphokines (e.g., alpha-interferon, gamma-interferon, platelet derived growth factor (PDGF), gCSF, gMCSF, TNF, epidermal growth factor (EGF), IL-1, IL-2, IL-4, IL-6, IL-8, IL-10, and IL-12). ABM2, AS01B, AS02, AS2A, Adjmer, Adjuvax, Algammulin, Alum, Aluminum phosphate, Aluminum potassium sulfate, Bordetella pertussis, Calcitriol, Chitosan, Cholera toxin, CpG, Dibutyl phthalate, Dimethyldioctadecylammonium bromide (DDA), Freund's adjuvant, Freund's complete, Freund's incomplete (IF A), GM-CSF, GMDP, Gamma Inulin Glycerol, HBSS (Hank's Balanced Salt Solution), Imiquimod, Interferon-Gamma, ISCOM, Lipid Core Peptide (LCP), Lipofectin, Lipopolysaccharide (LPS), Liposomes, MF59, MLP+TDM, Monophosphoryl lipid A, Montanide IMS-1313, Montanide ISA 206, Montanide ISA 720, Montanide ISA-51, Montanide ISA-50, nor-MDP, Oil-in-water emulsion, P1005 (non-ionic copolymer), Pam3Cys (lipoprotein), Pertussis toxin, Poloxarner, QS21, RaLPS, Ribi, Saponin, Seppic ISA 720, Soybean Oil, Squalene, Syntex Adjuvant Fornulation (SAF), Synthetic polynucleotides (poly IC/poly AU), TiterMax Tomatine, Vaxfectin, Xtendil, and Zyrosan.

Any route of administration can be used to deliver the composition to the mammal. Indeed, although more than one route can be used to administer the composition, a particular route can provide a more immediate and more effective reaction than another route. In some examples, the composition is administered via intramuscular injection, for example, using a syringe or needleless delivery device. In this respect, this disclosure also provides a syringe or a needleless delivery device comprising the composition. The pharmaceutical composition also can be applied or instilled into body cavities, absorbed through the skin (e.g., via a transdermal patch), inhaled, ingested, topically applied to tissue, or administered parenterally via, for instance, intravenous, peritoneal, or intraarterial administration.

The composition can be administered in or on a device that allows controlled or sustained release, such as a sponge, biocompatible meshwork, mechanical reservoir, or mechanical implant. Implants (see, e.g., U.S. Pat. No. 5,443,505), devices (see, e.g., U.S. Pat. No. 4,863,457), such as an implantable device, e.g., a mechanical reservoir or an implant or a device comprised of a polymeric composition, are particularly useful for administration of the composition. The composition also can be administered in the form of a sustained-release formulation (see, e.g., U.S. Pat. No. 5,378,475) comprising, for example, gel foam, hyaluronic acid, gelatin, chondroitin sulfate, a polyphosphoester, such as bis-2-hydroxyethyl-terephthalate (BHET), and/or a polylactic-glycolic acid.

The dose of the composition administered will depend on a number of factors, including the size of a target tissue, the extent of any side-effects, the particular route of administration, and the like. The dose ideally comprises an “effective amount” of the composition, e.g., a dose of composition, which provokes a desired immune response in the mammal. The desired immune response can entail production of antibodies, protection upon subsequent challenge, immune tolerance, immune cell activation, and the like. One dose or multiple doses of the composition can be administered to a mammal to elicit an immune response with desired characteristics, including the production of specific antibodies, or the production of functional T-cells.

In some embodiments, the method includes administering a treatment to the subject, thereby eliciting an immune response and treating the tumor in the subject in need thereof.

Methods can include any known treatment used to control tumor growth, size, metastasis or other desired tumor activity or characteristic. In some examples, treatment can include radiation, surgical removal or administration of a composition such as a composition including one or more antibodies as well as known CPI inhibitors.

Effective dosages and schedules for administering the compositions may be determined empirically, and making such determinations is within the skill in the art. The dosage ranges for the administration of the compositions are those large enough to produce the desired effect in which the symptoms/disorder are/is affected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 mg/kg to up to 100 mg/kg of body weight or more per day, depending on the factors mentioned above.

### V. Kits

The present disclosure also provides kits for detecting and monitoring MSI as well as for treating MSI-H cancer/tumor types. Such kits may include one or more arrays, compositions, vials or tools for sample collection and/or instructions for use in accordance with any of the methods, systems or compositions described herein. Instructions supplied in the kits of the embodiments provided herein are typically written instructions on a label or package insert (e.g., a paper sheet included in the kit), but machine-readable instructions (e.g., instructions carried on a magnetic or optical storage disk) are also acceptable. Instructions may be provided for practicing any of the methods or with the systems or compositions described herein.

Embodiments of the kits described herein are in suitable packaging. Suitable packaging includes, but is not limited to, vials, bottles, jars, flexible packaging (e.g., sealed Mylar or plastic bags), and the like. Kits may optionally provide additional components such as interpretive information. Normally, the kit comprises a container and a label or package insert(s) on or associated with the container. In some embodiments, provided herein are articles of manufacture comprising contents of the kits described above.

The following examples illustrate certain embodiments of the present disclosure, but should not be construed as limiting its scope in any way. Certain modifications and variations will be apparent to those skilled in the art from the teachings of the foregoing disclosure and the following examples, and these are intended to be encompassed by the spirit and scope of the disclosure. For example, the following examples provide data for assessing MSI status in humans. It is contemplated that the disclosed methods can be used for other subjects including dogs (possibly other animals) as MSI has been detected in dog cancers. Accordingly, the methods and systems disclosed herein can be used to screen for MSI status in dogs, by using an array with dog FSPs on it.

### EXAMPLES

### Example 1

This example provides a method to determine the accuracy of a disclosed Frameshift Peptide (FS) MSI/dMMR diagnostic.

One hundred samples from both MSI-H and 200 from MSS/MSI-L from colon cancer patients. These samples have been validated by standard immunohistochemistry (IHC). From a blinded set of 30 samples, the specificity, sensitivity and positive predictive value (PPV) of the assay will be determined. One can also determine the assay reproducibility across multiple wafer production runs to establish assay reproducibility.

In particular, 100 MSI-H, 20 MSI-L and 200 MSS samples will be purchased from Indivumed. MSI-L and MSS are considered the same relative treatment by current protocols. Since the majority of cancers, even in colon cancer, will be MSS (85%), it is desirable to capture the variability in this population with as many samples as reasonable to run.

Each sample will be diluted and assayed on Calviri manufactured FSP microarrays according to published methods (see, Stafford P, Cichacz Z, Woodbury N W, Johnston S A. Immunosignature system for diagnosis of cancer. Proc Natl Acad Sci USA. 2014; 111(30):E3072-80. doi: 10.1073/pnas.1409432111. PubMed PMID: 25024171; PMCID: PMC4121770, which is hereby incorporated by reference in its entirety). This method has been modified specifically to take in account the differences between IMS and FSP arrays. Unlike the binding in IMS, the reaction is incubated overnight in order to come to equilibrium with cognate binding sites. The slides are then washed in large volumes for up to an hour to release any avidity based (IMS) binding.

Two types of data analysis will be evaluated. First, is the analytic method which has been used for IMS microarrays. This has been used for approximately 50 different disease conditions and approximately 9,000 samples. In this workflow, a training set of samples is used to determine the p-value for each FSP feature for difference from the mean. A one-tail t-test (with error correction) is used to determine which FSP features are significantly different between case and control (in this case, MSI-H versus MSS/MSI-L), or an ANOVA for multiple distinctions. The number of peptides chosen is based on maximal independent cross-validation performance. At least 5 different classification algorithms are used and a simple voting method for class prediction. The cross-validation detects any non-disease classification associations, such as (for example) colon cancer leftside or rightside diagnoses. Typically, the classifier contains 100-500 peptides of the 400K. Once a set of features is determined, a fresh set of blinded, independent patient samples are used.

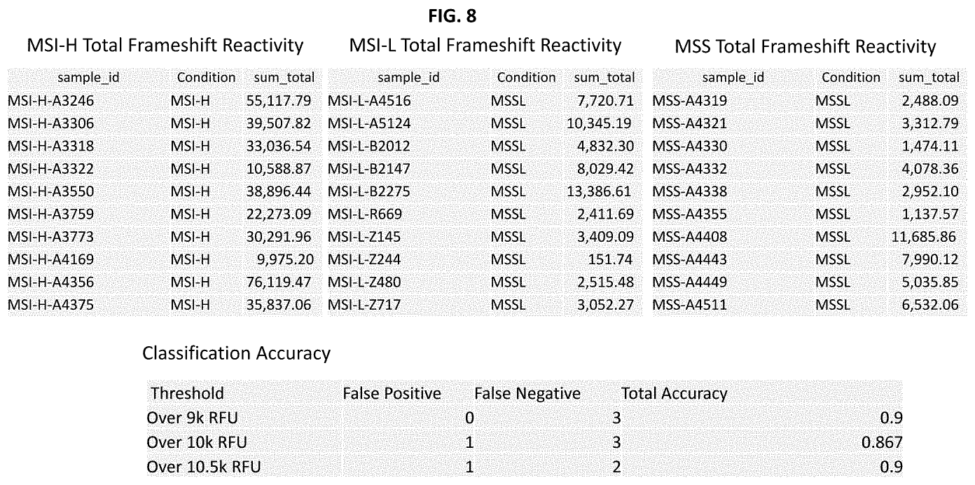

IMS data is near-normally distributed, lending itself to the t-test approach. However, the FSP microarray data is non-normally, patchy distributed. That is, for IMS the inventors can find peptides that have significantly higher fluorescence in almost all the cases versus the control samples. However, for the FSP microarrays, they found that peptides that are low in almost all the non-cancer samples but are significantly higher in some of the cancer patients (e.g. 10%), while in another 10% of cancer patients it will be a different set of peptides. This type of distribution lends itself to a simpler method leverages the fact that case cohorts have cumulatively more reactivity to frameshift antigens than non-case controls. Here, the peptides are selected by measuring the average reactivity of median-normalized data from the case cohort and comparing to the control cohort. We select those peptides that are minimally 5-fold higher in case than control. These peptide intensity values are summed per sample. The individual peptides can be used and/or all the peptides that comprise a full FSP can be used. If a blinded sample is at least two-fold higher than the mean control signal, it is considered a case sample. There are hybrid analytical methods that use the statistical feature selection methods from method #1 and the prediction methods from #2, and vice versa. The two basic approaches can be complementary of supportive. The performance of this form of the counting method is shown in FIG. 8.

The defined methods (ones already in the document) can be performed or calculated using individual distinct features/peptides on the array. However, since many frameshifts are represented or measured by multiple individual separate peptides on the array, an individual skilled in the art can also combine the information for distinct peptides on an array by use methods including, but not limited to the sum of the intensities from each peptide to create an aggregate measure of reactivity for a frameshift generally. The analysis methods disclosed herein can be performed either 1) at the level of individual peptides on the array, 2) an aggregate measure of frameshift reactivity across its constituent of one or more peptides on the array or 3) a combination of both 1 and 2 respectively.

A second assessment relative to a commercial product will be the reproducibility across wafers. If all late stage cancers were screened it could potentially require 500K arrays per year to meet demand in the US. While the major, early technical challenge for the commercialization of the immunosignature arrays was the reproducibility across wafers, this process is highly consistent now. The same 20 MSI-H and MSS samples will be tested on at least 3 different wafers and expect >90% reproducibility (as measured by R2) across all wafers.

If the assay performs with less than 90% specificity/sensitivity, one solution would be to only require distinction between MSI-H versus (MSI-L,MSS) rather than three classifications. As treatment is only recommended for MSI-H, this would not be a clinical limitation for the use of FSP microarray assay. A second is to use fresh blood samples to increase assay performance. While purchase samples from Indivumed will be used, collaborations with clinical partners who could be a source for additional, freshly drawn samples are being pursued. It is possible that reproducibility could be <90% reproducibility between wafers.

The disclosed assay is contemplated as being able to replace the current colon cancer MSI-H assays. However, the biggest clinical need is in testing other cancer types. The questions then are 1) whether the FSP array is useful as an MSI diagnostic on other cancers and 2) is the MSI signature the same between cancers. If the same FSPs cannot be used for other cancers, it would require developing an MSI signature for each cancer. Importantly, if the Feature Counting method is effective it would not require having a common signature peptide set.

To determine if cancers other than colon require a different classifier, at least 30 samples of endometrial cancer of each MSI status will be assayed and the FSP signature compared to that of colon cancer. In particular, 30 samples of endometrial cancer for each of the MSI states from Indivumed. The assays will be performed the same as described above. First, data will be analyzed to determine the peptide set by t-test that best discriminates the MSI state. The overlap with the colon cancer set will be evaluated. If the peptide sets are largely coincident, it indicates that the same FSP signature can be used for all cancers. If not, data will be pooled from all the colon and endometrial cancer samples to select FSPs that can distinguish all classes for all 3 states. If this is the case, it indicates that one may have to reselect the peptide classifier as more cancers are added for diagnosis. Thus, this example allows one to determine the feasibility of extending the FS peptide MSI assay to all cancers.

IMS arrays can also be used to diagnose MSI-H and MSIS/MSI-L patients. IMS arrays are peptide arrays of 10-330K peptides. However, the peptides are chosen from random sequence space to maximize chemical diversity. The antibodies binding the peptides do so as mimotopes with low affinity, unlike the FSP binding which is high affinity, cognate binding. The peptides are closer together on the IMS arrays to enhance avidity (FIGS. 2A-2C). The rationale is that mutations caused by MSI would produce neoantigens that would induce antibodies which could indirectly reflect the MSI status.

The procedure for assaying the MSI status by IMS is essentially the same as in using the FSP arrays. However, the process for analyzing the intensity data from the arrays can be different. For example, for IMS always uses a two-way t-test to determine which peptides are significantly more or less fluorescent in the MSI-H versus MSS/MSI-L or an ANOVA comparison to distinguish all three states. For the FSP arrays, the informative features are generally higher in the case versus controls and the distribution is more stochastic. Therefore, an additive analysis is more appropriate.

### Example 2

This example demonstrates ability of a FSP microarray to be used to determine the MSI status of a patient.

Utilized in this method is a peptide microarray which contains FSPs, such a peptide microarray containing all possible FSPs that could be produced by a tumor via indels in MSs in coding regions. There are approximately 7,000 such MSs with monobase runs longer than 7 in the human genome which could produce approximately 14,000 FSPs greater than 10aa long. These peptides could be produced by errors in DNA replication or by transcription through the MSs. Errors in transcription are -100 times more frequent than in DNA replication. Additionally, the FSP microarray can contain all possible exon mis-splicing events that would be predicted to create FSP longer than l0aa. There are approximately 220,000 such FSPs. Mis-splicing is more error prone in tumors and even more so in MSI tumors. This is probably because out of the approximately 120 proteins involved in splicing, 21 of them have MSs in the coding regions. Indels in any of these could disrupt the splicing process. Thus, the FSP act as a receptor for all errors in tumors.

In this example, a FSP microarrays of 400K, 15aa peptides can be used to represent all the MSs and mis-splicing FSPs 10aa or longer. Standard photolithography systems with silicon wafers and masks can be used as illustrated in FIGS. 2A and 2B. The chemistry is BOC peptide synthesis with a photoacid activator. 208 arrays are produced on each 300 mm silicon wafer. This type of system has been published (Legutki J B, Zhao Z G, Greving M, Woodbury N, Johnston S A, Stafford P. Scalable high-density peptide arrays for comprehensive health monitoring. Nat Commun. 2014; 5:4785. doi: 10.1038/ncomms5785. PubMed PMID: 25183057, which is hereby incorporated by references in its entirety).

A fundamental principle of the assay is that tumors elicit antibodies to FSPs and these antibodies can be detected by a simple ELISA-like assay on the FSP microarrays. A biopsy of the tumor is not needed. The system is very sensitive to the presence of a tumor because any tumor produced FSP that activates a B-cell amplifies the antibody signal up to 1011 fold in one week.

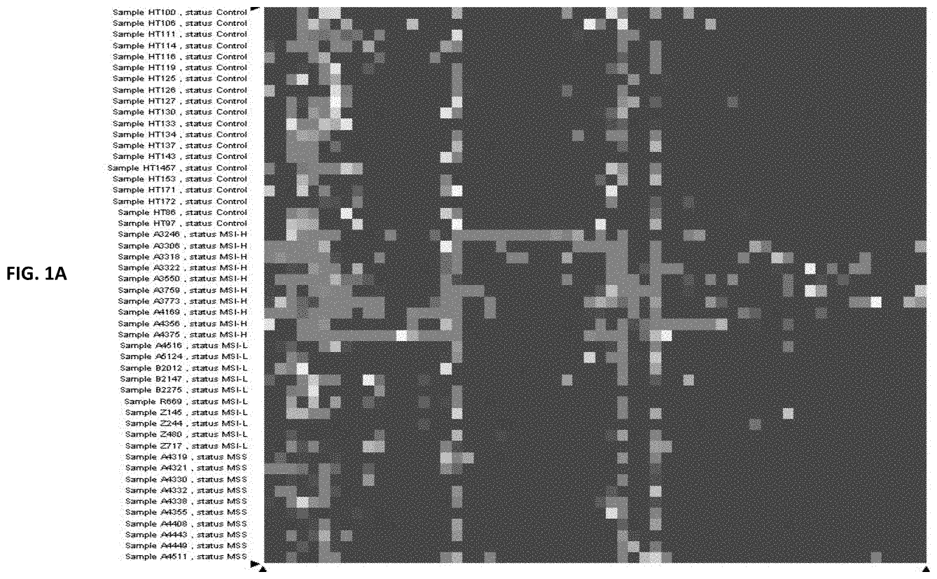

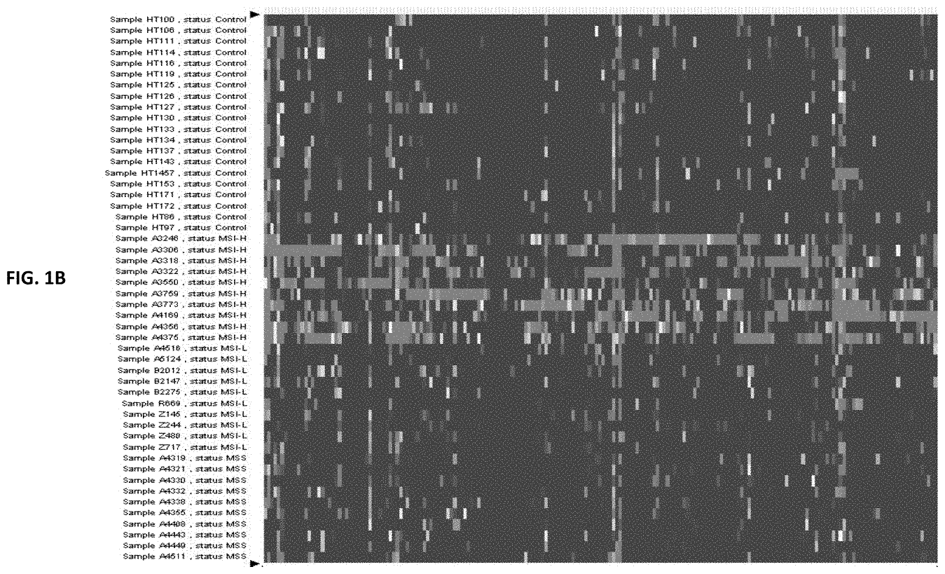

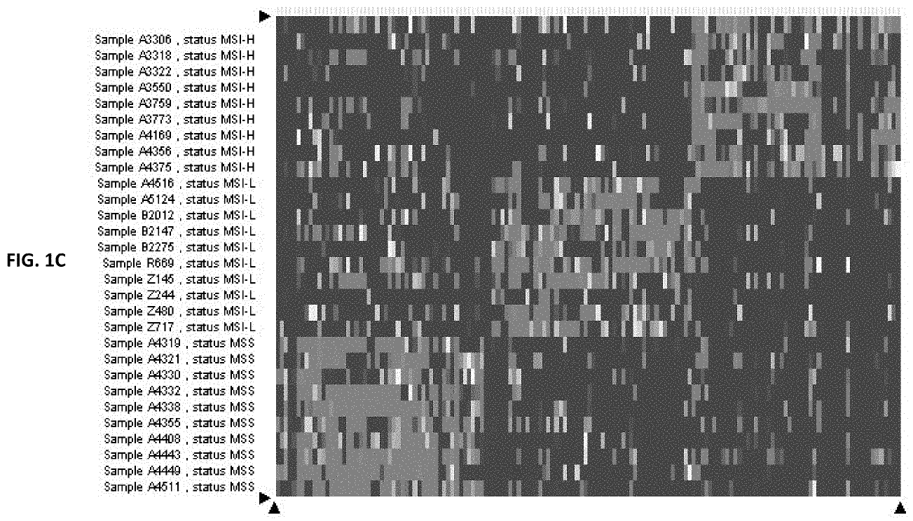

In FIG. 1C, the same FSP microarray platform is used to distinguish MSI-H, MSI-L, and MSS samples from colon cancer patients. These gold-standard samples were purchased from Indivumed (GMBh) and were assayed for MSI status using the standard PCR diagnostic. There were 10 samples of each class. Though the total sample numbers are low, the FSP microarray identified enough significantly different peptides to classify the 3 types in leave-one out analysis at 100% accuracy. Interestingly, FSPs from mis-splicing were better classifiers than those from MSs. It has been noted that MSI-H has increased mis-splicing. From the same assay, these colon cancer samples could be distinguished from non-cancer subjects using a classifier based on different peptides than those FSPs used to distinguish MSI status. The implication of this result is that if FSP microarrays were used to diagnose colon cancer, the same data could be used to distinguish MSI status.

MSI-H have much higher reaction to a set of the FS peptides on the array than do MSI-L or normal samples (FIGS. 1A-1C, 8). All 3 states can be distinguished. These figures demonstrate that both the FSP generated from MS and those from the exon mis-splicing can be used to perform the diagnosis. The exon FSP yielded a better performance. When all 400K peptides were used and the best 100 classifier peptides chosen, all were from exon mis-splicing.

Two specific analytical methods can be applied:

1) Using the median-normalized intensity data from the frameshift peptide array, a one-sided t-test is performed between case and control. Using a p-value cutoff for significance, a fixed number of peptides are selected as predictors. A cross-validation using SVM or other appropriate machine learning algorithm (some working examples: Naïve Bayes, k-nearest neighbor, decision trees and linear and non-linear discriminate analysis) will enable a prediction of accuracy. Once peptides are selected from this training process, blinded samples are tested which provides an absolute performance estimate of class prediction.

2) A simpler method leverages the fact that case cohorts have cumulatively more reactivity to frameshift antigens than non-case controls. Here, the peptides are selected by measuring the average reactivity of median-normalized data from the case cohort and comparing to the control cohort. Peptides that are minimally 5-fold higher in case than control are selected. These peptide intensity values are summed per sample. If a blinded sample is at least two-fold higher than the mean control signal, it is considered a case sample. There are hybrid analytical methods that use the statistical feature selection methods from method #1 and the prediction methods from #2, and vice versa. Typically, the difference in cross-validation performance is minor, and the two methods are simply supportive of the predictive power of the raw data.

Immunosignature (IMS) arrays can also be used to diagnose MSI-H and MSI-L patients. IMS arrays are peptide arrays of 10-330K peptides. However, the peptides are chosen from random sequence space to maximize chemical diversity. The antibodies binding the peptides do so as mimotopes with low affinity, unlike the FSP binding which is high affinity, cognate binding. The peptides are closer together on the IMS arrays to enhance avidity (FIG. 2C). The rationale is that mutations caused by MSI would produce neoantigens that would induce antibodies which could indirectly reflect the MSI status.

The procedure for assaying the MSI status by IMS is essentially the same as in using the FSP arrays. However, the process for analyzing the intensity data from the arrays can be different. For example, for IMS generally a two-way t-test is used to determine which peptides are significantly more or less fluorescent in the MSI-H versus MSI-L or an ANOVA comparison to distinguish all three states. For the FSP arrays, the informative features are generally higher in the case versus controls and the distribution is more stochastic. Therefore, an additive analysis is more appropriate.

### Example 3

This example illustrates additional testing of a simple array based antibody detection system to detect MSI.

Immunocheckpoint therapeutics (CPI) have been remarkably effective in patients that have defects in mismatch repair (dMMR). This defect is manifested by insertions and deletions (INDEL) in microsatellites (MS). If the MS is in a coding region the INDEL can create a frameshift peptide (FS). These FS peptides are highly immunogenic and may at least in part explain the strong response to CPI treatment. The response has been so predictable that the FDA approved the use of PD-1 solely on the basis of the patient having an MS instability high (MSI-H) diagnosis. MSI-H is frequent in colon, endometrial and stomach cancer (15-28%) and patients with these cancers are often tested for the condition. MSI-H does occur infrequently at least 20 other cancers. Reports of remarkable responses in these rare MSI-H patients argues for screening all metastatic patients for MSI. Current approaches to screening for MSI (immunohistochemistry, PCR sequencing of MSs and NGS of exons) require tumor tissue. For wide spread screening of metastatic patients it would be an advance to have a blood based screening technology. Here we tested two types of peptides arrays for screening antibodies as an approach to MSI diagnosis. Both methods suggest such an assay is feasible.

Cancer treatment is undergoing a dramatic shift with the introduction of checkpoint inhibitor therapeutics. A clear theme arising from the analysis of responders versus non-responders is that the response depends on the number and quality of the neoantigens produced by mutations. This is very evident with regard to patients with dMMR. Mutations or methylation variants in repair genes lead to INDELS particularly in MS. Homopolymer runs of nucleotides (e.g., 15 As) are particularly sensitive to slippage in replication and therefore creating INDELS. INDELS in MS in a coding region will produce a downstream FS peptide that could be highly immunogenic. This is probably why MSI-H patients respond so well to CPI therapy. Colon, endometrial and stomach cancers are MSI-H at 15-28% frequency and therefore these cancers are frequently screened for MSI. Screens of up to 29 other cancers by NGS of MS regions has demonstrated low (0-5%) frequency of MSI-H. However, these infrequent MSI cases also can experience remarkable responses. This has been the basis of suggestions that all metastatic cancers be screened for MSI. Here we explore serological diagnostics for MSI that would facilitate such widespread screens.

Currently there are three basic approaches to MSI diagnosis. The dMMR defect often produces a deletion of one of 6 proteins involved in the repair process. This can be detected by immunocytochemical staining in tumor sections. Absence of two or more proteins is scored as MSI-H. A second approach is to PCR and analyze 6 long, homopolymer, MS runs. An INDEL in 1 MS is scored as MSI-Low and 2 or more as MSI-H. Increasingly, the NGS of tumors, or recently of cell free DNA in blood (11), is including a programmed analysis of 1000 MS to make an MSI diagnostic. All three current methods require biopsy tissue from the tumor and in some cases, matching non-tumor tissue which can be problematic. As MSI-H tumors are hypermutant, including frequent FS neoantigens, and produce more INDELs in coding MS, we reasoned that the patient might create enough antibodies to the neoantigens to be distinguished as MSI-H.

IMS diagnostics are one approach to broadly surveying the antibodies in a person. Arrays containing 125-330K, 12-17aa peptides are synthesized using photolithography and Boc peptide chemistry. The peptides are chosen from random sequence space to maximize chemical diversity. Since any particular antibody will bind a mimotope, the affinity is generally low affinity and the antibody retained by avidity. As the MSI-H condition produces FS peptides, we explored a second type of peptide array. All possible FS peptides greater than 10aa long were informatically predicted that could arise from INDELS in MS in coding regions, or from mis-splicing of an exon. Both types of arrays were tested against MSI-H, MSI-L, MSS and non-cancer sera samples.

### Example 4

The FSPs can also be used in a vaccine to therapeutically treat an individual with an MSI-H tumor. It has been demonstrated that peptides that are reactive on the arrays can be used as vaccines (9,10). Further we have shown that the level of protection in mouse tumor models positively correlates with the amount of antibody binding on the array (9). Since many of the peptides are reactive in patients with MSI-H tumors, it is contemplated that a vaccine can be pre-made consisting of one or more of the 23 peptides in Table 3, such as all 23 peptides, for example. They could be administered as peptides, in nanoparticles or encoded by DNA plasmids (gene vaccines), viral vectors or mRNA to the patient. The vaccine could be administered at the same or different time with an adjuvant (e.g., Hiltonol) and/or an immunotherapeutic (e.g., checkpoint inhibitor). Since each MSI-H patient is reactive to multiple of the 23 FSPs, a protective immune response would be induced. It is also contemplated that a vaccine can include one or more components from the list in Table 1. Alternatively, a personal vaccine consisting of only the peptides that individual was reactive to could be constructed. This type of personal vaccine would take time to make and would cost more than the pre-made vaccine.

### Results

Samples: 10 MSI-H, 10 MSH-L and 10 MSI-S serum samples from patients with colorectal cancer were purchased from Invidumed (Hamburg, Germany). Patient ages ranged from 29 to 83, nine females and 21 males with malignant neoplasm of the colon, either sigmoid (13), ascending (4), descending (1), hepatic flexure (1), transverse (3), rectum (3) or caecum (2). Patients provided serum at diagnosis. Patients were classified using the standard 6 panel MS (BAT 25, BAT 26, BAT 40, D17S250, D2S123 and D5S346). The MSI-Ls scored positive on 1 MS. The MSI-Hs scored positive on 2-6 MSs. The 18 samples from adult non-cancer subjects were from a panel of blood donor samples.

Arrays: The IMS arrays consisted of 125K peptides 12-17aa long. They are synthesized in-situ using mask-based photochemistry. There are 24 arrays per standard microscope slide. The peptides are chosen by an algorithm to represent the maximum 5aa space. They have essentially no similarity to human proteome sequence space. Therefore antibodies bind the peptides as mimotopes of the actual peptide that elicited the antibody.

The FSP arrays consist of FS peptides bioinformatically chosen as being at least 10aa long. These are not naturally occurring peptides in normal cells. They could result from INDELs in MSs in coding regions (˜14K) or from mis-splicing of exons (˜200K). The peptides synthesized on the array were 15aa long. If a FS peptide was longer than 15aa long in was represented by overlapping peptides if less than 30aa or including non-overlapping peptides if longer than 30aa. These arrays were synthesis having 400K peptides representing the -220K FS peptides. The arrays were synthesized to specifications by Nimblegen by published methods.

IMS Array Analysis: The 48 samples were assayed as described in the Methods section. Briefly, the sera was diluted, applied to an array, washed and the antibodies detected with labelled secondary antibody against IgG. The assay is essentially that of standard ELISAs. The arrays were scanned to obtain the florescent intensity for each peptide. After aligning the arrays against mask files, each peptide was assigned an intensity score. The intensity of each array was median normalized and the data analyzed using a two-way t-Test to identify peptides that have significantly different intensities, or ANOVA analysis in 4 way comparisons.

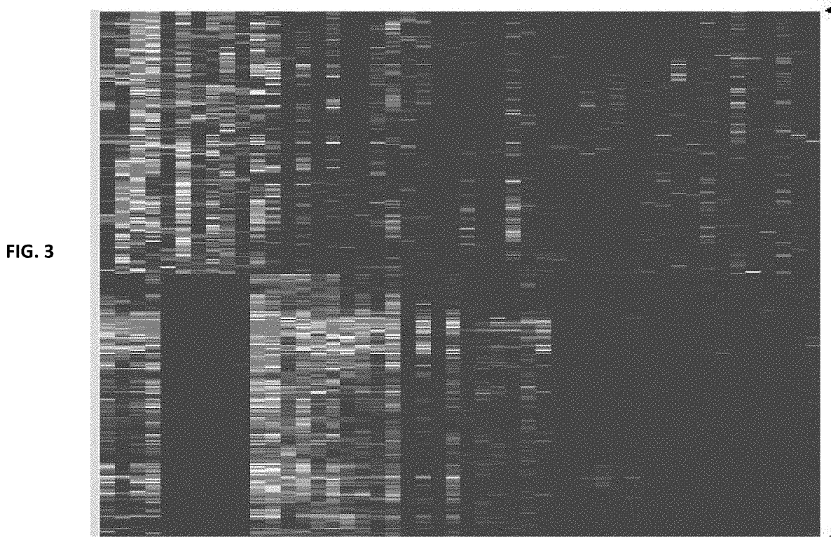

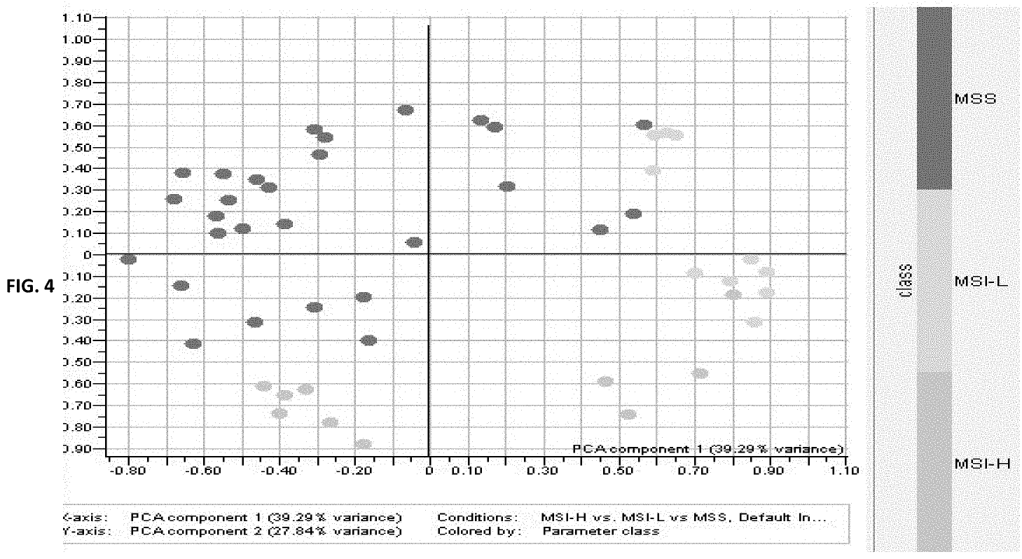

As represented in FIG. 3, the IMS was ˜82% accurate in designating the classes using leave one out cross validation. 100 peptides were used as the classifier. The PCA of the analysis is shown in FIG. 4. 2 MSS patients were called as MSI-L, 1 control as MSI-H and 4 MSI-H as MSI-L. Relative to the clinical application, 50% of the MSI-H were not called as such.

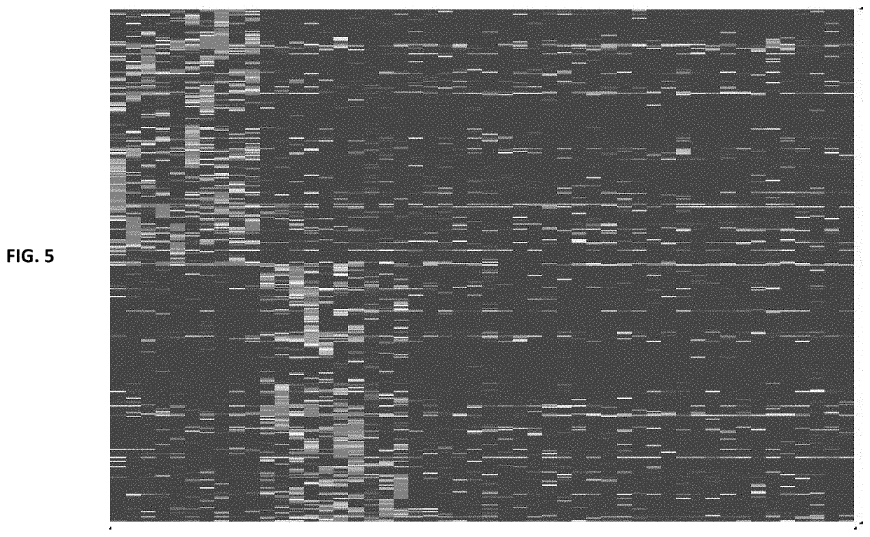

FSP Array Analysis: The same set of samples were analyzed by a very similar procedure on the FSP arrays. We first restricted the analysis to the FS peptides associated with the MSs. 100 peptides were chosen as the classifier. As shown in FIG. 5, the distinction of the 3 classes of samples was 100% accurate by leave one out cross validation.

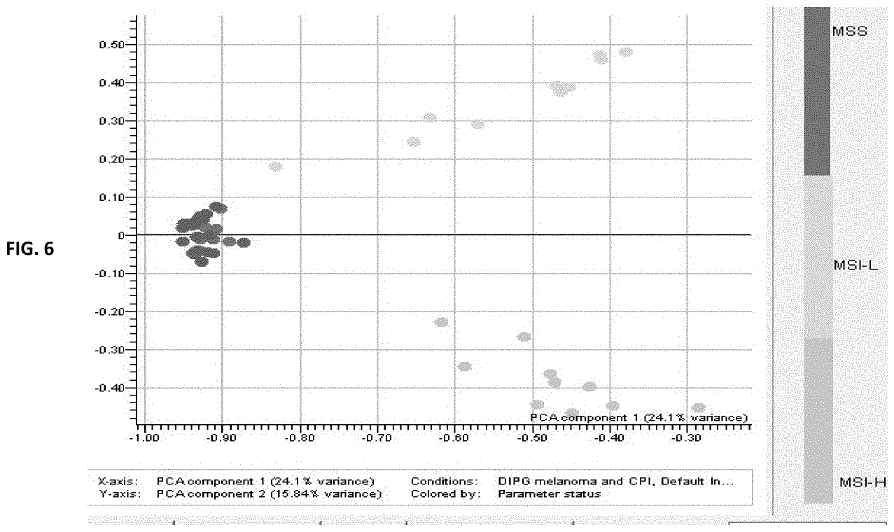

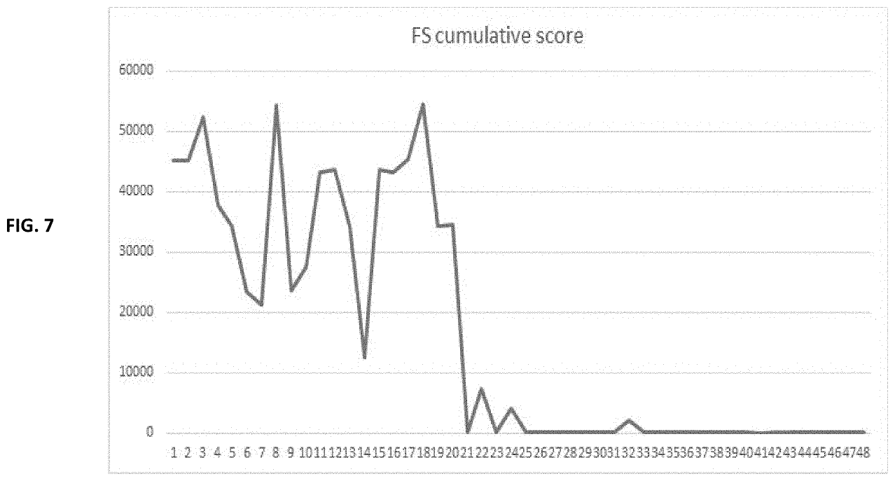

Using all the peptides to select the top performers resulted in 100 peptides that were all from exon mis-splicing (FIG. 6). In leave-one out cross validation the distinction was also 100% accurate, as evident in the PCA graph in FIG. 7. As another approach we calculated the mean, normalized intensity for all the features for each subject. Interestingly, both the MSI-H and MSI-L had much higher total fluorescence than the MSS and non-cancer samples. However, total fluorescence could not distinguish MSI-H from MSI-L.

We tested two types of arrays for the potential of antibody-based classification of MSI status. The IMS arrays, containing peptides from random sequence space, classified the MSI-H from MSS/non-cancer with 82% accuracy but only 50% sensitivity. The FSP arrays, containing FS peptides that could be generated from INDELs in MSs or exon mis-splicing, performed better on the same samples. Using only the MS FS to classify the accuracy was 100% in distinguishing the MSI-H, MSI-L and MSS/non-cancer samples. When all the FS peptides (MS plus exon) were used to classify, the best 100 peptides were all exon associated FS. These peptides also had 100% accuracy in leave one out testing. A simple adding of total florescent intensity was able to distinguish MSI-H+MSI-L from MSS/non-cancer samples but not MSI-H from -L.

One limitation with the currently available methods require obtaining tumor DNA which is not always possible and much less convenient than a blood based assay. A second is that a MSI-H designation does not always correspond to a positive CPI therapeutic response (30-90% positive responders). Apparently, the current assays for MSI status cannot make this discrimination. The FSP arrays is contemplated to be able to.

Patients that have MSI-H tumors have a high response rate to CPI treatment. This was the basis for the precedent setting approval of Keytruda for multiple tumors diagnosed as MSI-H. Many analyses of responders versus non-responders indicate several factors may underlay the difference—including PDL-1 expression by the tumor, tumor mutational burden and MSI-status. TMB and MSI-status are the best indicators as they most directly relate to the mutations in the tumor. MSI status is the best indicator to date. This is probably for several reasons. First, tumors with MSI-H have by definition high levels of INDELs in MS. These create FS peptides that are foreign to the immune system and therefore more likely to be highly immunogenic, compared to single amino acid changes. TMB counts all neoantigens, most of which are single amino acid changes. Second, the MS instability makes the tumor hypermutational, creating more immunogenic targets. For example, of the genes involved in exon splicing, some have MSs in them. This may be the cause of the much higher exon mis-splicing in MSI-H tumors. Mis-splicing of exons will also produce FS peptides. Third, it has been reported that in MSI-H tumors there are more INDELs in coding MSs, which will create more FS peptides. Given these considerations we argue that the higher production of FS peptides in these tumors could account for the exception CPI response rate. This contention is supported by the observation that renal cancers, that have low TMB but unusually high FS peptide production, have a high response rate to CPI therapy.

The ideal method to predict CPI response would be to measure the immune directly measure the immune response to the tumor. TMB and MSI analysis as practiced only indirectly measure this. The most practical way to do this would be by measuring antibodies. It was reasoned that if MSI-H tumors are creating a large number of neoantigens, particularly FS peptides, they may elicit antibodies to them. First tested was an established peptide array system, immunosignature diagnostic, to test this proposal. As presented in FIGS. 3 and 4, this system could distinguish with 82% accuracy in leave one out cross validation, but with only 50% sensitivity. This performance is not as high as the exiting assays, though it is much more convenient in not requiring tumor tissue.

The MSI-H tumors may be creating many more FS peptides. IMS would detect all antibodies, not just to FS peptides, and would only bind them as mimotopes. It was reasoned, therefore, that by measuring antibodies to the FS peptides directly the assay would improve. The created arrays included all FS peptides (>10aa) predicted downstream of mononucleotide MS (>7 bases). They also included all possible FS peptides (>10aa) predicted from possible mis-splicing events of exons. The logic for including exon FS peptides was the observation that tumors in general, and MSI-H tumors in particular, have much higher mis-splicing rates. This analysis produced a total of 220K peptides that could be represented by -400K, 15aa peptides. When the same sample set used on the IMS array was used on this array the performance was substantially better (FIGS. 4, 5), with 100% accuracy in leave one out cross validation. The FS peptides predicted to be generated by INDELs in MS were predictive, but, interestingly, the FS peptides from exons were as predictive. This may be due to the destabilization of splicing in MSI tumors.

In FIG. 8, another method of diagnosis is illustrated which is accomplished by summing the florescent intensity for each set of 15aa peptides on the arrays that constitute a particular FSP. For example, a FSP of 45aa may be represented by 3 15aa peptides. A patient may have reactivity to 2 of the 3 peptides. The reactivity of the 2 is summed. The total fluorescence on this set of peptides is used to classify MSI-H from MSS.

The FS peptide array results can be used as diagnostics. First, they can replace or at least augment the screening of high MSI-H incident cancers. If the FS peptide arrays perform at or better than existing screens it offers the advantage of the simplicity of a blood based assay and possibly be less expensive. A second advantage, stemming from the simplicity and less cost, is to encourage screening of patients with low frequency of MSI-H. Many cancer types have 1-5% MSI-H frequencies, yet these patients are probably just as likely to have a positive response to CPI therapy. It is contemplated that the disclosed assays and methods would be beneficial to screen all metastatic patients for MSI status.

It is contemplated that the high performance may be due to directly measuring antibodies generated to the FS peptides. Since these arrays directly measure the immune reactivity to the tumor antigens, they can be used to assess whether responding and non-responding MSI-H individuals have discriminating profiles on the arrays. The disclosed methods and systems can be used to indicate which FS peptides are good candidates for vaccines. As such, vaccines including identified FS peptides are disclosed as well.

In view of the many possible embodiments to which the principles of the embodiments disclosed herein may be applied, it should be recognized that the illustrated embodiments are only preferred examples and should not be taken as limiting the scope. Rather, the scope of embodiments as described herein is defined by the following claims.

### REFERENCES (EACH OF WHICH IS HEREBY INCORPORATED BY REFERENCE IN ITS ENTIRETY)

1 Collura, A. et al. Patients with colorectal tumors with microsatellite instability and large deletions in HSP110 T17 have improved response to 5-fluorouracil-based chemotherapy. Gastroenterology 146, 401-411 e401, doi:10.1053/j.gastro.2013.10.054 (2014).
2 Le, D. T. et al. Mismatch repair deficiency predicts response of solid tumors to PD-1 blockade. Science 357, 409-413, doi:10.1126/science.aan6733 (2017).
3 Le, D. T. et al. PD-1 Blockade in Tumors with Mismatch-Repair Deficiency. NEngl J Med 372, 2509-2520, doi:10.1056/NEJMoa1500596 (2015).
4 Bonneville, R. et al. Landscape of Microsatellite Instability Across 39 Cancer Types. JCO Precis Oncol 2017, doi:10.1200/PO.17.00073 (2017).
Wang, Y., Shi, C., Eisenberg, R. & Vnencak-Jones, C. L. Differences in Microsatellite Instability Profiles between Endometrioid and Colorectal Cancers: A Potential Cause for False-Negative Results? J Mol Diagn 19, 57-64, doi:10.1016/j.jmoldx.2016.07.008 (2017).
6 Ryan, E., Sheahan, K., Creavin, B., Mohan, H. M. & Winter, D. C. The current value of determining the mismatch repair status of colorectal cancer: A rationale for routine testing. Crit Rev Oncol Hematol 116, 38-57, doi:10.1016/j.critrevonc.2017.05.006 (2017).
7 Salipante, S. J., Scroggins, S. M., Hampel, H. L., Turner, E. H. & Pritchard, C. C. Microsatellite instability detection by next generation sequencing. Clin Chem 60, 1192-1199, doi:10.1373/clinchem.2014.223677 (2014).
8. McNeil, E., K. L. Griffin, A. M. Mellett, N. J. Madrill and J. R. Mickelson. Microsatellite instability in canine mammary gland tumors. J. Vet. Intern. Med. 21:1034-1040 (2007)
9. Zhang, J., L. Shen and S. A. Johnston. Using frameshift peptide arrays for cancer neo-antigen screening. Scientific Reports, 2018. 81:p 1-10.
10. Shen, L., J. Zhang, H. Lee, M. T. Batista and S. A. Johnston. RNA transcription and splicing errors as a source of cancer frameshift neoantigens for vaccines. Scientific Reports, 2019 (in press).
11. Georgeadis, A. et al. Noninvasive Detection of Microsatellite Instability and High Tumor Mutation Burden in Cancer Patients Treated with PD-1 Blockade. Clinical Cancer Research. 2019. DOI. 10.1158/1078-0432.CCR-19-1372

What is claimed is:

1. A method of identifying microsatellite instability status, comprising:
applying an antibody containing fluid sample to a frameshift peptide array comprising peptides selected from frameshifts created by insertion or deletions in microsatellites and/or frameshifts created by mis-splicing of exons and associated with microsatellite instability (MSI), wherein the peptides have the sequence with 8 or more contiguous amino acids of the frameshift sequences provided in Tables 1 and 2; and
analyzing binding of the antibody to the peptides associated with MSI, thereby identifying microsatellite instability status of the sample by comparing the relative binding of antibodies to the peptides on the array.

2. The method of claim 1, wherein the array comprises one or more peptides having the sequence with 8 or more contiguous amino acids of the frameshift sequences according to SEQ ID NOs: 81-155.

3. The method of claim 1, wherein the array consists essentially of peptides having the sequence with 8 or more contiguous amino acids of the frameshift sequences according to one of SEQ ID NOs: 81-155.

4. The method of claim 1, wherein the array consists of peptides having sequences with 8 or more contiguous amino acids of the frameshift sequences according to SEQ ID NOs: 81-155.

5. The method of claim 1, wherein the peptides are 8-30 amino acids in length.

6. The method of claim 2, wherein analyzing comprises adding total fluorescent values of each peptide comprising the frameshift and counting those above a threshold to effect a diagnosis.

7. The method of claim 1, further comprising obtaining the antibody containing fluid sample from a subject.

8. The method of claim 7, wherein the subject is a human.

9. The method of claim 7, wherein the subject is a dog.

10. The method of claim 1, wherein the antibody containing fluid sample is blood, plasma or saliva.

11. The method of claim 1, wherein analyzing comprises classifying the sample as MSI-high, MSI-low or MS-Stable.

12. The method of claim 11, wherein detection of high MSI indicates the sample would respond to checkpoint inhibitor (CPI) immunotherapy.

13. The method of claim 12, further comprising administering an CPI immunotherapy to the subject with high MSI.

14. The method of claim 12, wherein CPI immunotherapy targets CTLA4, PD-1, and/or PD-L1.

15. A method of identifying microsatellite instability status (MSI), comprising:
applying an antibody containing fluid sample to an ELISA comprising peptides selected from frameshifts created by insertion or deletions in microsatellites and/or frameshifts created by mis-splicing of exons and associated with microsatellite instability (MSI), wherein the peptides have the sequence with 8 or more contiguous amino acids of the frameshift sequences provided in Table 3; and
analyzing binding of the antibody to the peptides associated with MSI, thereby identifying microsatellite instability status of the sample by comparing the relative binding of antibodies to the peptides.

16. The method of claim 15, wherein the ELISA comprises peptides set forth in Table 3 with SEQ ID NOs: 3, 5, 8, 9, 11, 12, 18, 19, 21, 24, 27, 32, 41, 44, 49, 52, 56, 63, 67, 68, 71 and 72 to identify MSI.

17. The method of claim 15, further comprising obtaining the antibody containing fluid sample from a subject.

18. The method of claim 17, wherein the subject is a human.

19. The method of claim 17, wherein the subject is a dog.

20. The method of claim 15, wherein the antibody containing fluid sample is blood, plasma or saliva.

21. The method of claim 15, wherein analyzing comprises classifying the sample as MSI-high, MSI-low or MS-Stable.

22. The method of claim 21, wherein detection of high MSI indicates the sample would respond to checkpoint inhibitor (CPI) immunotherapy.

23. The method of claim 22, further comprising administering an CPI immunotherapy to the subject with high MSI.

24. The method of claim 22, wherein CPI immunotherapy targets CTLA4, PD-1, and/or PD-L1.

25. A vaccine, comprising:
one or more peptides having the sequence according to one provided in Tables 1 and/or 2 and/or a nucleic acid capable of expressing the one or more peptides and a pharmaceutically acceptable carrier.

26. The vaccine of claim 25, further comprising an adjuvant.

27. The vaccine of claim 25, wherein the vaccine comprises one or more vectors expressing the peptides according to Tables 1 and/or 2.

28. The vaccine of claim 27, wherein the amino acid sequences are separated by a peptide linker.

29. A method of treating and/or inhibiting a MSI-H cancer or tumor in a subject, comprising
administering to the subject the vaccine of claim 25.

30. The method of claim 29, wherein the method further comprises administering an additional therapeutic agent.

31. The method of claim 30, wherein the additional therapeutic agent is a CPI.

32. A method of eliciting an immune response in a subject with a MSI-H cancer or tumor, comprising
administering to the subject the vaccine of claim 25.

33. A composition comprising:
one or more peptides having the sequence according to one of SEQ ID NOs: 3, 5, 8, 9, 11, 12, 18, 19, 21, 24, 27, 32, 41, 44, 49, 52, 56, 63, 67, 68, 71 and 72 and/or a nucleic acid capable of expressing the one or more peptides.

34. The composition of claim 33, wherein the amino acid sequences are separated by a peptide linker.
